(19) United States
(12) Patent Application Publication
(10) Pub. No.: US 2025/0197376 A1
(43) Pub. Date: Jun. 19, 2025

(54) PYRAZINE AMIDE DERIVATIVES

(71) Applicant: Novartis AG, Basel (CH)

(72) Inventors: Richard Yichong HUANG, El Cerrito, CA (US); Thomas Martin KIRRANE, JR., San Ramon, CA (US); Vanessa MARX, El Cerrito, CA (US); Anne-Catherine MATA, Berkeley, CA (US); Christopher Ronald SARKO, San Ramon, CA (US); Benjamin Robert TAFT, Martinez, CA (US); Fumiaki YOKOKAWA, Dublin, CA (US)

(21) Appl. No.: 18/849,215

(22) PCT Filed: Mar. 22, 2023
(86) PCT No.: PCT/IB2023/052827

§ 371 Date: Sep. 20, 2024

Related U.S. Application Data
(60) Provisional application No. 63/269,879, filed on Mar. 24, 2022.

(51) Int. Cl. C07D 403/04; A61K 31/497; A61K 31/5377; A61K 45/06; A61P 33/06; C07D 401/14; C07D 403/14; C07D 405/14; C07D 413/14; C07D 471/04; C07D 495/04

(52) U.S. Cl. CPC C07D 403/04; A61K 31/497; A61K 31/5377; A61K 45/06; A61P 33/06; C07D 401/14; C07D 403/14; C07D 405/14; C07D 413/14; C07D 471/04; C07D 495/04

(57) ABSTRACT

The present invention relates to pyrazine amide derivative compounds, such as those according to formula (I) and compositions including said compounds, The invention also provides such pyrazine amide derivative compounds for use in the treatment of plasmodium related diseases such as malaria.

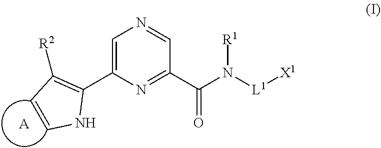

### FIELD OF THE INVENTION

The invention provides pyrazine amide derivative compounds and compositions including said compounds. The invention also provides such pyrazine amide derivative compounds for use in the treatment of parasitic related diseases such as malaria.

### BACKGROUND OF THE INVENTION

Malaria is an infectious disease caused by four protozoan parasites: Plasmodium falciparum; Plasmodium vivax; Plasmodium ovale; and Plasmodium malaria. These four parasites are typically transmitted by the bite of an infected female Anopheles mosquito. Malaria is a problem in many parts of the world and over the last few decades the malaria burden has steadily increased. An estimated 1-3 million people die every year from malaria-mostly children under the age of 5. This increase in malaria mortality is due in part to the fact that Plasmodium falciparum, the deadliest malaria parasite, has acquired resistance against nearly all available antimalarial drugs, even resistance to artemisinins is emerging.

International patent applications WO 2011/143419 disclose various pyrazines for use as ataxia telangiectasia mutated (ATM) and Rad3 related (together, ATR) kinase inhibitors. However, for the treatment of malaria, there is a need for targeted therapies which may be selective (i.e. may inhibit a certain targeted molecule more selectively as compared to other molecular targets, e.g. ATR kinases, e.g. as described hereinafter), which may have the benefit of reducing side effects and may also have a benefit that malaria can be treated selectively.

In view of the foregoing, there remains a need to develop novel compounds as selective antiparasitic agents. The invention provides such compounds, pharmaceutically acceptable salts thereof, solid forms thereof, pharmaceutical compositions thereof and combinations thereof. The invention further provides methods of treating, preventing, or ameliorating parasitic disease, comprising administering to a subject in need thereof an effective amount of a compound of the invention.

### SUMMARY OF THE INVENTION

According to one aspect of the invention, there is hereby provided a compound of formula (I), or a pharmaceutically acceptable salt thereof:

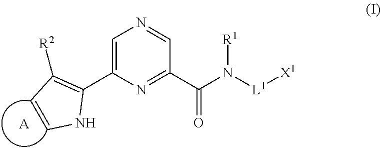

wherein:
R¹ is i) H or ii) C₁-C₃alkyl;
the moiety:

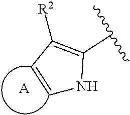

is selected from the group consisting of:

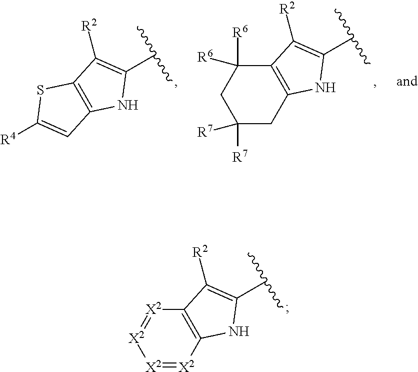

R² is i) C₁-C₃alkyl, ii) halo, iii) hydrogen, iv) C₁-C₃haloalkyl or v) cyano;
each X² is independently selected from the group consisting of N and CR³, with the proviso that at least one X² is CR³;
each R³ is independently selected from the group consisting of hydrogen, halo, SF₅, C₁-C₃alkyl, hydroxyl, cyano, O—C₁-C₃alkyl, SO₂—C₁-C₃alkyl, C(O)O—C₁-C₃alkyl, O—C₁-C₃haloalkyl, C₁-C₃haloalkyl, CH₂NH₂, OCH₂C₆H₅, C(O)—N(H)—C₁-C₄alkylene-NH₂ and

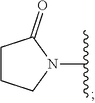 [SMILES: *N1CCCC1=O]

R⁴ is C₁-C₃alkyl, C(O)N(R⁵)₂ or CO₂C₁-C₃alkyl;
each R⁵ is independently H or C₁-C₃alkyl;
each R⁶ is H or together the two R⁶ groups form oxo;
each R⁷ is independently selected from the group consisting of H and C₁-C₃alkyl;
L¹ is i) absent or ii) C₁-C₅alkylene, optionally substituted with OH or C₃-C₆cycloalkyl;
X¹ is i) H,
   ii) OH,
   iii) NH₂,
   iv) a) C₃-C₆cycloalkyl substituted with a NH₂ substituent, b) C₃-C₆cyclohaloalkyl substituted with a NH₂ substituent or c) 4-6 membered heterocyclyl comprising one heteroatom selected from O and N, said 4-6 membered heterocyclyl substituted with NH₂, or

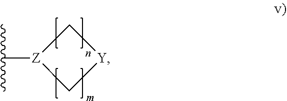

wherein Z is N or CH, Y is O or NH, n is 1 or 2 and m is 1 or 2;
provided that when L¹ is absent, X¹ is not H, OH or NH₂.

According to a second aspect of the invention, there is hereby provided the compound or pharmaceutically acceptable salt thereof according to the first aspect of the invention for use as a medicament.

According to a third aspect of the invention, there is hereby provided the compound or pharmaceutically acceptable salt thereof according to the first aspect of the invention for use in treating a plasmodium related disease.

According to a fourth aspect of the invention, there is hereby provided a use of the compound or pharmaceutically acceptable salt thereof according to the first aspect of the invention in the manufacture of a medicament for treating a Plasmodium related disease.

According to a fifth aspect of the invention, there is hereby provided a method of treating a Plasmodium related disease, the method comprising administering to a subject in need thereof, a therapeutically effective amount of the compound or pharmaceutically acceptable salt thereof according to the first aspect of the invention.

According to a sixth aspect of the invention, there is hereby provided a pharmaceutical composition comprising the compound or pharmaceutically acceptable salt thereof according to the first aspect of the invention, and one or more pharmaceutically acceptable carriers.

### DETAILED DESCRIPTION OF THE INVENTION

The invention therefore provides the following numbered embodiments. It will be recognized that features specified in each embodiment may be combined with other specified features to provide further embodiments of the present invention.

Embodiment 1. A compound of formula (I), or a pharmaceutically acceptable salt thereof:

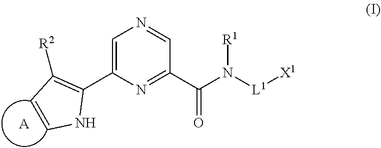

wherein:
R¹ is i) H or ii) C₁-C₃alkyl;
the moiety:

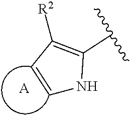

is selected from the group consisting of:

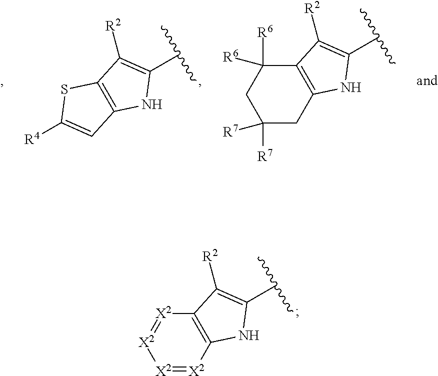

R² is i) C₁-C₃alkyl, ii) halo, iii) hydrogen, iv) C₁-C₃haloalkyl or v) cyano;
each X² is independently selected from the group consisting of N and CR³, with the proviso that at least one X² is CR³;
each R³ is independently selected from the group consisting of hydrogen, halo, SF₅, C₁-C₃alkyl, hydroxyl, cyano, O—C₁-C₃alkyl, SO₂-C₁-C₃alkyl, C(O)O—C₁-C₃alkyl, O—C₁-C₃haloalkyl, C₁-C₃haloalkyl, CH₂NH₂, OCH₂C₆H₅, C(O)—N(H)—C₁-C₄alkylene-NH₂ and

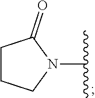 [SMILES: *N1CCCC1=O]

R⁴ is C₁-C₃alkyl, C(O)N(R⁵)₂ or CO₂C₁-C₃alkyl;
each R⁵ is independently H or C₁-C₃alkyl;
each R⁶ is H or together the two R⁶ groups form oxo;
each R⁷ is independently selected from the group consisting of H and C₁-C₃alkyl;
L¹ is i) absent or ii) C₁-C₅alkylene, optionally substituted with OH or C₃-C₆cycloalkyl;
X¹ is i) H,
   ii) OH,
   iii) NH₂,
   iv) a) C₃-C₆cycloalkyl substituted with a NH₂ substituent, b) C₃-C₆cyclohaloalkyl substituted with a NH₂ substituent or c) 4-6 membered heterocyclyl comprising one heteroatom selected from O and N, said 4-6 membered heterocyclyl substituted with NH₂, or

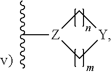

wherein Z is N or CH, Y is O or NH, n is 1 or 2 and m is 1 or 2;
provided that when L¹ is absent, X¹ is not H, OH or NH₂.

Embodiment 3. The compound or pharmaceutically acceptable salt thereof according to any of the embodiments herein, wherein at least two X² are CR³.

Embodiment 4. The compound or pharmaceutically acceptable salt thereof according to any of the embodiments herein, wherein at least three X² are CR³.

Embodiment 5. The compound or pharmaceutically acceptable salt thereof according to any of the embodiments herein, wherein R¹ is H.

Embodiment 6. The compound or pharmaceutically acceptable salt thereof according to any of the embodiments herein wherein L¹ is C₁-C₅alkylene, optionally substituted with OH or C₃cycloalkyl.

Embodiment 7. The compound or pharmaceutically acceptable salt thereof according to any of the embodiments herein wherein L¹ is unsubstituted C₁-C₅alkylene.

Embodiment 8. The compound or pharmaceutically acceptable salt thereof according to any of the embodiments herein wherein L¹ is unsubstituted C₄alkylene.

Embodiment 9. The compound or pharmaceutically acceptable salt thereof according to any of the embodiments herein wherein X¹ is:
i) NH₂,
ii) a) C₃-C₆cycloalkyl substituted with NH₂, b) C₃cyclohaloalkyl substituted with NH₂ or c) 4-6 membered heterocyclyl comprising one heteroatom selected from O and N, said 4-6 membered heterocyclyl being substituted with NH₂, or

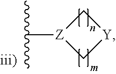

wherein Z is CH, Y is NH, n is 1 or 2 and m is 1 or 2.

Embodiment 10. The compound or pharmaceutically acceptable salt thereof according to any of the embodiments herein wherein X¹ is NH₂.

Embodiment 11. The compound or pharmaceutically acceptable salt thereof according to any of the embodiments herein, wherein the moiety:

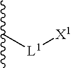

is selected from:

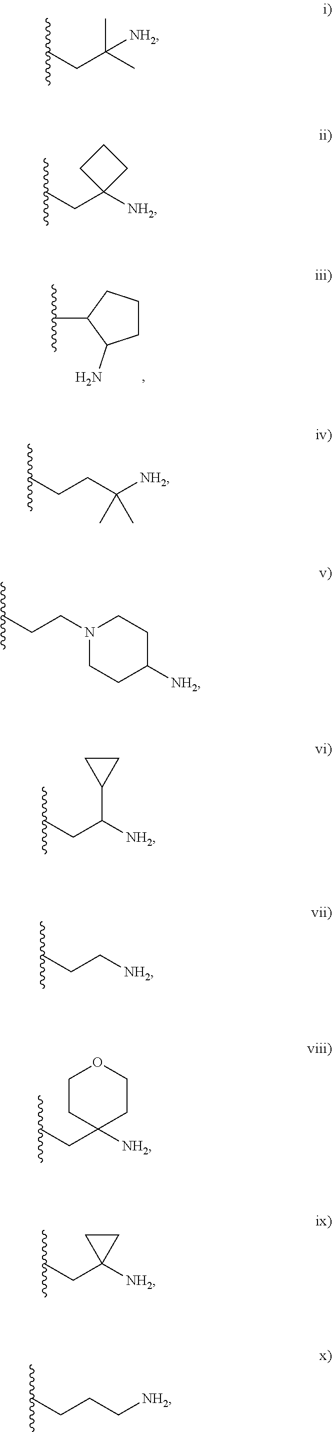

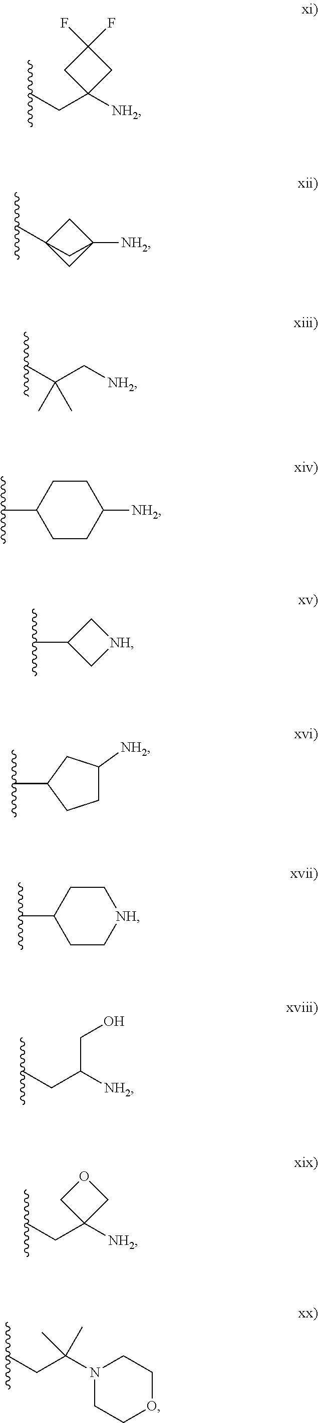

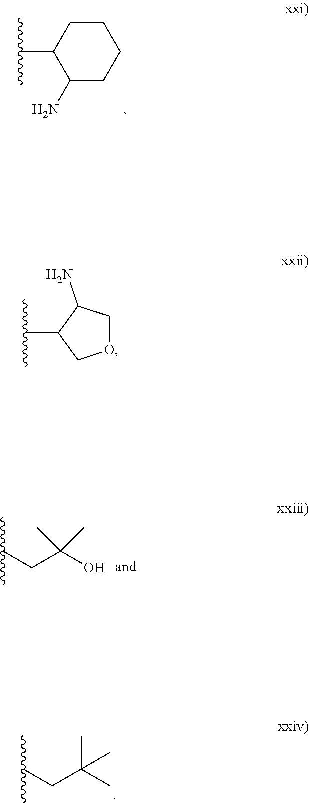

Embodiment 12. The compound or pharmaceutically acceptable salt thereof according to any of the embodiments herein, wherein the moiety:

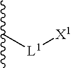

is

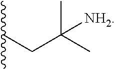 [SMILES: *CC(C)(C)N]

Embodiment 13. The compound or pharmaceutically acceptable salt thereof according to any of the embodiments herein, wherein R² is CH₃.

Embodiment 14. The compound or pharmaceutically acceptable salt thereof according to any of the embodiments herein, wherein the moiety:

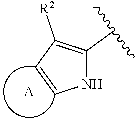

is

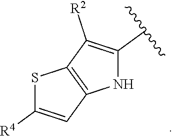

Embodiment 15. The compound or pharmaceutically acceptable salt thereof according to any of the embodiments herein wherein the moiety:

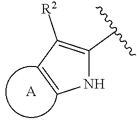

is selected from the group consisting of:

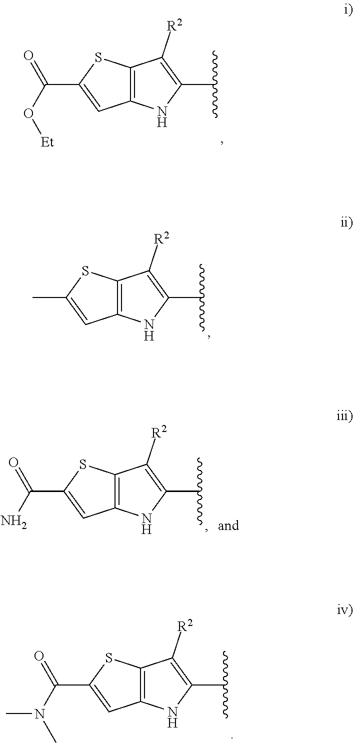

Embodiment 16. The compound or pharmaceutically acceptable salt thereof according to any of the embodiments herein, wherein the moiety:

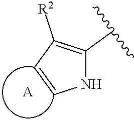

is

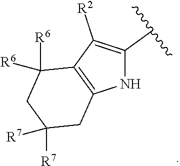

Embodiment 17. The compound or pharmaceutically acceptable salt thereof according to any of the embodiments herein wherein the moiety:

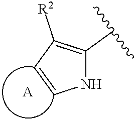

is selected from the group consisting of:

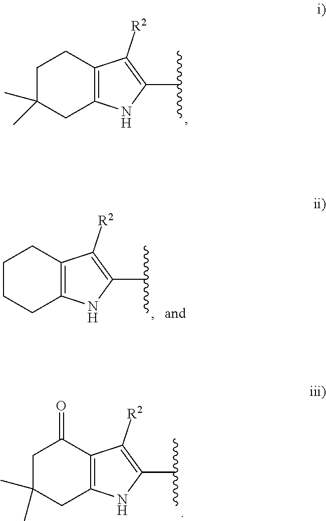

Embodiment 18. The compound or pharmaceutically acceptable salt thereof according to any of the embodiments herein, wherein the moiety:

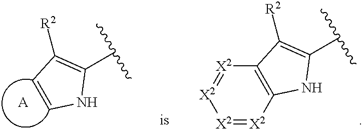

Embodiment 19. The compound or pharmaceutically acceptable salt thereof according to any of the embodiments herein wherein the moiety:

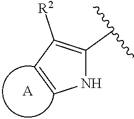

is selected from the group consisting of:

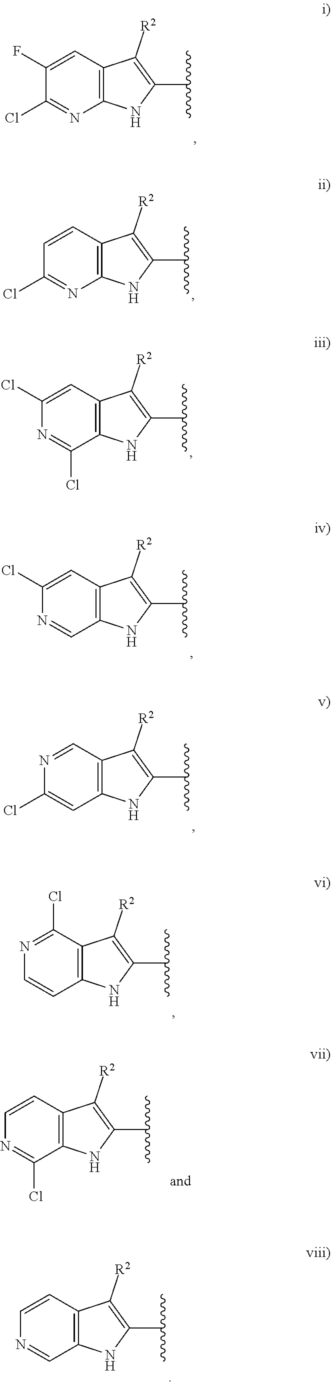

Embodiment 20. The compound or pharmaceutically acceptable salt thereof according to any of the embodiments herein, wherein the moiety:

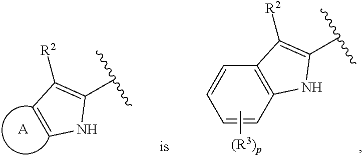

p is 0, 1, 2, 3 or 4, and wherein each R³ is independently selected from the group consisting of halo, SF₅, methyl, hydroxyl, cyano, OMe, SO₂Me, C(O)OMe, O—C₁haloalkyl, C₁haloalkyl, CH₂NH₂, OCH₂C₆H₅, C(O)—N(H)—C₁-C₄alkylene-NH₂ and

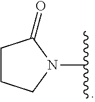 [SMILES: *N1CCCC1=O]

Embodiment 21. The compound or pharmaceutically acceptable salt thereof according to any of the embodiments herein wherein p is 0, 1 or 2.

Embodiment 22. The compound or pharmaceutically acceptable salt thereof according to any of the embodiments herein wherein p is 1 or 2.

Embodiment 23. The compound or pharmaceutically acceptable salt thereof according to any of the embodiments herein wherein each R³ is independently selected from the group consisting of halo, OC₁haloalkyl, SF₅, methyl and C(O)OMe.

Embodiment 24. The compound or pharmaceutically acceptable salt thereof according to any of the embodiments herein wherein the moiety:

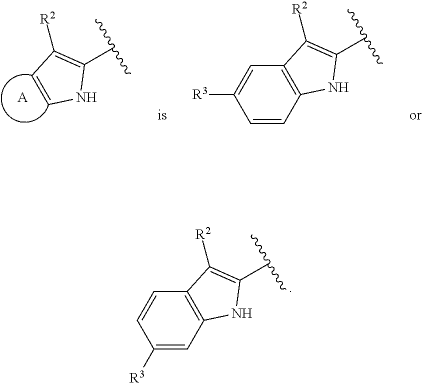

Embodiment 25. The compound or pharmaceutically acceptable salt thereof according to any of the embodiments herein wherein the moiety:

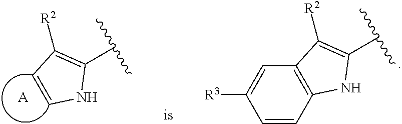

Embodiment 26. The compound or pharmaceutically acceptable salt thereof according to any of the embodiments herein wherein R³ is halo, OCF₃, SF₅, or OCHF₂.

Embodiment 27. The compound or pharmaceutically acceptable salt thereof according to any of the embodiments herein wherein R³ is SF₅.

Embodiment 28. The compound or pharmaceutically acceptable salt thereof according to any of the embodiments herein wherein the moiety:

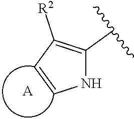

is selected from the group consisting of:

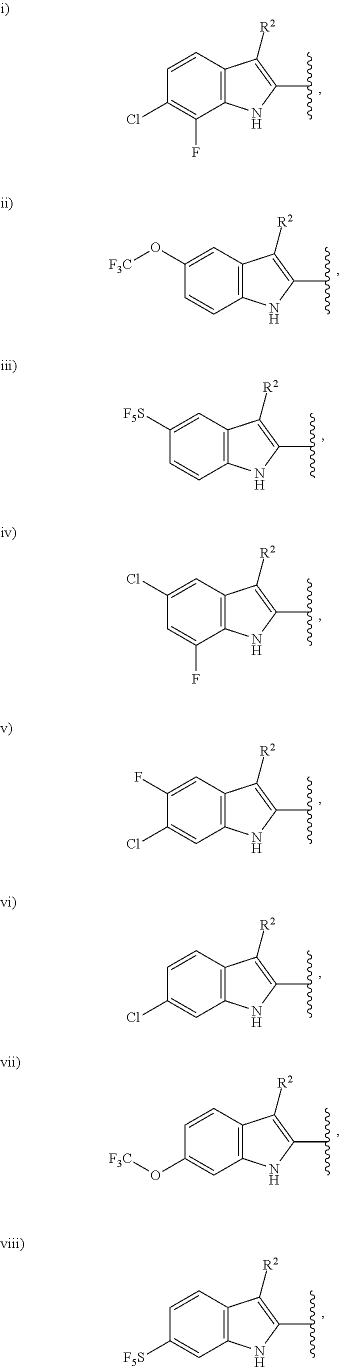

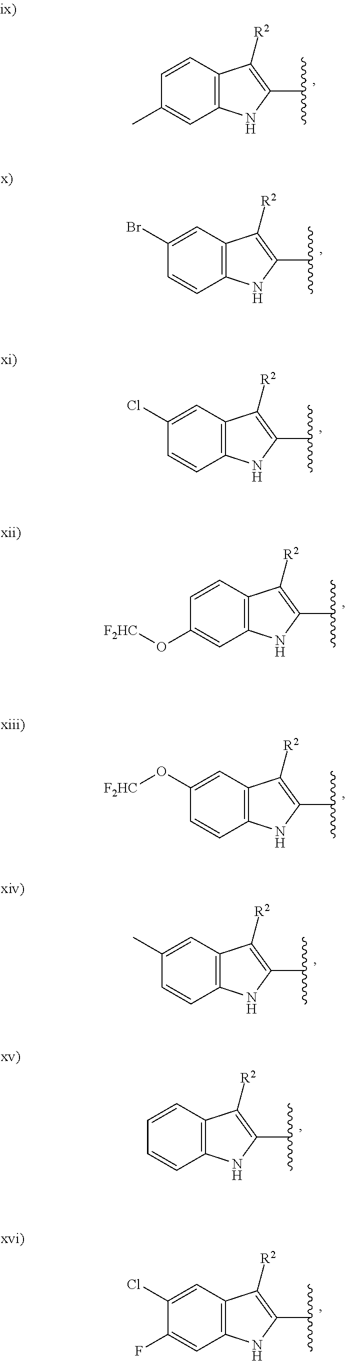

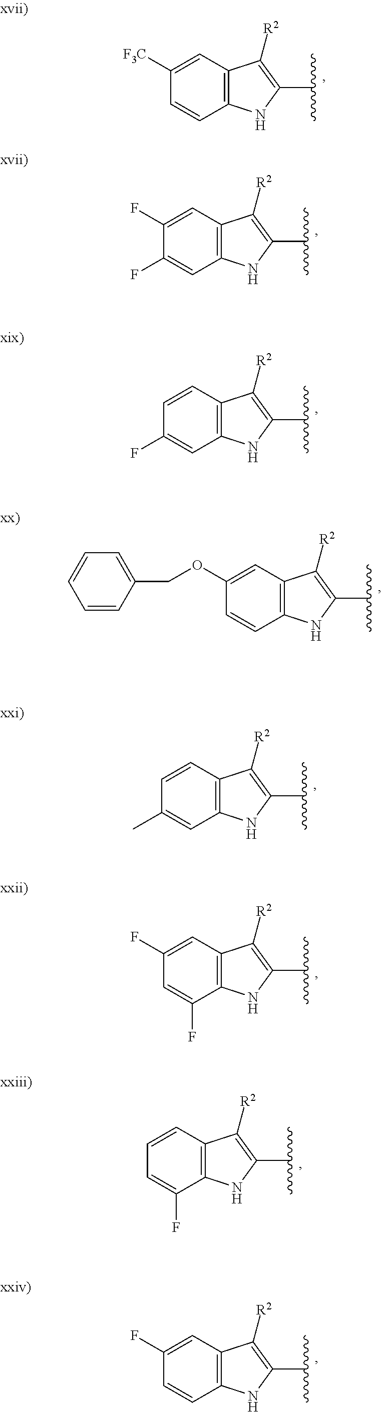

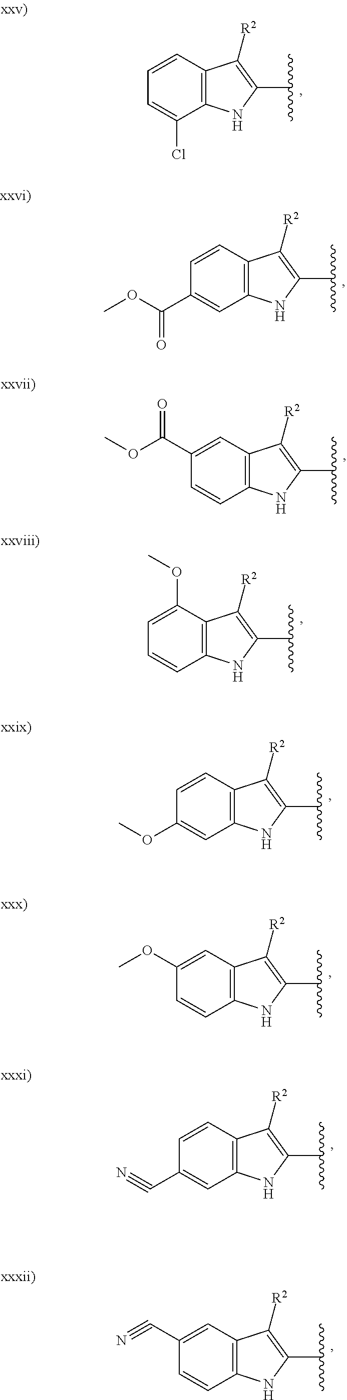

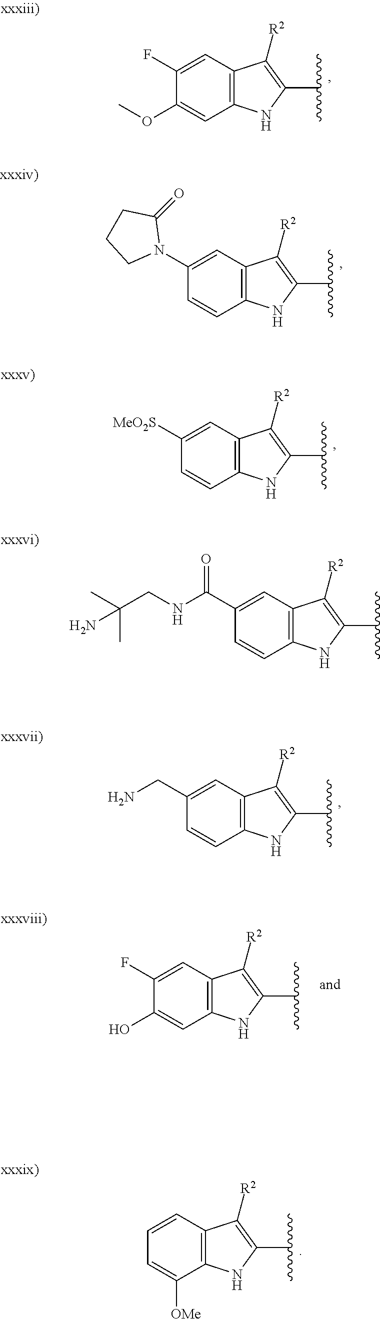

Embodiment 29. The compound according to any of the embodiments herein selected from the group consisting of:
N-(2-amino-2-methylpropyl)-6-(6-chloro-7-fluoro-3-methyl-1H-indol-2-yl)pyrazine-2-carboxamide;
N-(2-amino-2-methylpropyl)-6-(3-methyl-5-(trifluoromethoxy)-1H-indol-2-yl)pyrazine-2-carboxamide;
N-(2-amino-2-methylpropyl)-6-(3-methyl-5-(pentafluoro-λ⁶-sulfaneyl)-1H-indol-2-yl)pyrazine-2-carboxamide;
N-(2-amino-2-methylpropyl)-6-(5-chloro-7-fluoro-3-methyl-1H-indol-2-yl)pyrazine-2-carboxamide;
N-(2-amino-2-methylpropyl)-6-(6-chloro-5-fluoro-3-methyl-1H-indol-2-yl)pyrazine-2-carboxamide;
N-(2-amino-2-methylpropyl)-6-(6-chloro-3-methyl-1H-indol-2-yl)pyrazine-2-carboxamide;
N-((1-aminocyclobutyl)methyl)-6-(3-methyl-5-(trifluoromethoxy)-1H-indol-2-yl)pyrazine-2-carboxamide;
N-(2-amino-2-methylpropyl)-6-(3-methyl-6-(trifluoromethoxy)-1H-indol-2-yl)pyrazine-2-carboxamide;
N-((1S,2S)-2-aminocyclopentyl)-6-(6-chloro-5-fluoro-3-methyl-1H-indol-2-yl)pyrazine-2-carboxamide;
N-(3-amino-3-methylbutyl)-6-(3-methyl-5-(trifluoromethoxy)-1H-indol-2-yl)pyrazine-2-carboxamide;
N-(2-amino-2-methylpropyl)-6-(3-methyl-6-(pentafluoro-λ⁶-sulfaneyl)-1H-indol-2-yl)pyrazine-2-carboxamide;
N-(2-amino-2-methylpropyl)-6-(5-chloro-6-fluoro-3-methyl-1H-indol-2-yl)pyrazine-2-carboxamide;
N-(2-amino-2-methylpropyl)-6-(3-chloro-5-(trifluoromethoxy)-1H-indol-2-yl)pyrazine-2-carboxamide;
N-(2-amino-2-methylpropyl)-6-(3,6-dimethyl-1H-indol-2-yl)pyrazine-2-carboxamide; Ethyl 5-(6-((2-amino-2-methylpropyl)carbamoyl)pyrazin-2-yl)-6-methyl-4H-thieno[3,2-b]pyrrole-2-carboxylate;
N-(2-amino-2-methylpropyl)-6-(5-bromo-3-methyl-1H-indol-2-yl)pyrazine-2-carboxamide;
N-(2-(4-aminopiperidin-1-yl)ethyl)-6-(6-chloro-5-fluoro-3-methyl-1H-indol-2-yl)pyrazine-2-carboxamide;
N-(2-amino-2-methylpropyl)-6-(6-chloro-7-fluoro-1H-indol-2-yl)pyrazine-2-carboxamide;
N-(2-amino-2-methylpropyl)-6-(5-chloro-3-methyl-1H-indol-2-yl)pyrazine-2-carboxamide;
N-(2-amino-2-methylpropyl)-6-(5-chloro-7-fluoro-1H-indol-2-yl)pyrazine-2-carboxamide;
N-(2-amino-2-methylpropyl)-6-(6-(difluoromethoxy)-3-methyl-1H-indol-2-yl)pyrazine-2-carboxamide;
N-(2-amino-2-cyclopropylethyl)-6-(6-chloro-5-fluoro-3-methyl-1H-indol-2-yl)pyrazine-2-carboxamide;
N-(2-amino-2-methylpropyl)-6-(3,6,6-trimethyl-4,5,6,7-tetrahydro-1H-indol-2-yl)pyrazine-2-carboxamide;
N-(2-amino-2-methylpropyl)-6-(5-(difluoromethoxy)-3-methyl-1H-indol-2-yl)pyrazine-2-carboxamide;
N-(2-amino-2-methylpropyl)-6-(5-(pentafluoro-λ⁶-sulfaneyl)-1H-indol-2-yl)pyrazine-2-carboxamide;
N-(2-aminoethyl)-6-(3-methyl-5-(trifluoromethoxy)-1H-indol-2-yl)pyrazine-2-carboxamide;
N-(2-amino-2-methylpropyl)-6-(6-chloro-5-fluoro-1H-indol-2-yl)pyrazine-2-carboxamide;
N-(2-amino-2-methylpropyl)-6-(3,5-dimethyl-1H-indol-2-yl)pyrazine-2-carboxamide;
N-((4-aminotetrahydro-2H-pyran-4-yl)methyl)-6-(3-methyl-5-(trifluoromethoxy)-1H-indol-2-yl)pyrazine-2-carboxamide;
N-((4-aminotetrahydro-2H-pyran-4-yl)methyl)-6-(6-chloro-5-fluoro-3-methyl-1H-indol-2-yl)pyrazine-2-carboxamide;
N-(2-amino-2-methylpropyl)-6-(6-(trifluoromethoxy)-1H-indol-2-yl)pyrazine-2-carboxamide;
(S)—N-(2-amino-2-cyclopropylethyl)-6-(3-methyl-1H-indol-2-yl)pyrazine-2-carboxamide;
Methyl 2-(6-((2-amino-2-methylpropyl)carbamoyl)pyrazin-2-yl)-3-methyl-1H-indole-5-carboxylate;
(R)—N-(2-amino-2-cyclopropylethyl)-6-(3-methyl-1H-indol-2-yl)pyrazine-2-carboxamide;
N-(2-amino-2-methylpropyl)-6-(3-isopropyl-5-(trifluoromethoxy)-1H-indol-2-yl)pyrazine-2-carboxamide;
N-((1-aminocyclopropyl)methyl)-6-(6-chloro-5-fluoro-3-methyl-1H-indol-2-yl)pyrazine-2-carboxamide;
N-((1-aminocyclobutyl)methyl)-6-(3-methyl-1H-indol-2-yl)pyrazine-2-carboxamide;
N-((1-amino-3,3-difluorocyclobutyl)methyl)-6-(6-chloro-5-fluoro-3-methyl-1H-indol-2-yl)pyrazine-2-carboxamide;
N-(2-amino-2-methylpropyl)-6-(2,6-dimethyl-4H-thieno[3,2-b]pyrrol-5-yl)pyrazine-2-carboxamide;
N-(2-amino-2-methylpropyl)-6-(3-chloro-1H-indol-2-yl)pyrazine-2-carboxamide;
N-(2-amino-2-methylpropyl)-N-methyl-6-(3-methyl-5-(trifluoromethoxy)-1H-indol-2-yl)pyrazine-2-carboxamide;
N-(2-amino-2-methylpropyl)-6-(5-chloro-6-fluoro-1H-indol-2-yl)pyrazine-2-carboxamide;
N-(2-amino-2-methylpropyl)-6-(5-(trifluoromethoxy)-1H-indol-2-yl)pyrazine-2-carboxamide;
N-(2-amino-2-methylpropyl)-6-(5-(trifluoromethyl)-1H-indol-2-yl)pyrazine-2-carboxamide;
N-(2-amino-2-methylpropyl)-6-(3-methyl-1H-indol-2-yl)pyrazine-2-carboxamide;
N-(2-amino-2-methylpropyl)-6-(5-chloro-1H-indol-2-yl)pyrazine-2-carboxamide;
N-(3-aminopropyl)-6-(3-methyl-5-(trifluoromethoxy)-1H-indol-2-yl)pyrazine-2-carboxamide;
N-(3-amino-3-methylbutyl)-6-(3-methyl-1H-indol-2-yl)pyrazine-2-carboxamide;
N-(2-amino-2-methylpropyl)-6-(3-ethyl-1H-indol-2-yl)pyrazine-2-carboxamide;
N-(2-amino-2-methylpropyl)-6-(3-methyl-4,5,6,7-tetrahydro-1H-indol-2-yl)pyrazine-2-carboxamide;
N-(2-amino-2-methylpropyl)-6-(5,6-difluoro-1H-indol-2-yl)pyrazine-2-carboxamide;
N-(2-amino-2-methylpropyl)-6-(3-isopropyl-1H-indol-2-yl)pyrazine-2-carboxamide;
N-(2-amino-2-methylpropyl)-6-(6-fluoro-1H-indol-2-yl)pyrazine-2-carboxamide;
N-(3-aminobicyclo[1.1.1]pentan-1-yl)-6-(3-methyl-5-(trifluoromethoxy)-1H-indol-2-yl)pyrazine-2-carboxamide;
N-(2-amino-2-methylpropyl)-6-(5-(benzyloxy)-1H-indol-2-yl)pyrazine-2-carboxamide;
N-(2-amino-2-methylpropyl)-6-(6-methyl-1H-indol-2-yl)pyrazine-2-carboxamide;
N-(2-amino-2-methylpropyl)-6-(5,7-difluoro-1H-indol-2-yl)pyrazine-2-carboxamide;
N-(2-amino-2-methylpropyl)-6-(7-fluoro-1H-indol-2-yl)pyrazine-2-carboxamide;
N-(2-amino-2-methylpropyl)-6-(5-methyl-1H-indol-2-yl)pyrazine-2-carboxamide;
N-(2-amino-2-methylpropyl)-6-(5-fluoro-1H-indol-2-yl)pyrazine-2-carboxamide;
Ethyl 5-(6-((2-amino-2-methylpropyl)carbamoyl)pyrazin-2-yl)-4H-thieno[3,2-b]pyrrole-2-carboxylate;
N-(2-aminoethyl)-6-(3-methyl-1H-indol-2-yl)pyrazine-2-carboxamide;
N-(3-aminobicyclo[1.1.1]pentan-1-yl)-6-(3-methyl-1H-indol-2-yl)pyrazine-2-carboxamide;
N-(1-amino-2-methylpropan-2-yl)-6-(3-methyl-1H-indol-2-yl)pyrazine-2-carboxamide;
N-(2-amino-2-methylpropyl)-6-(7-chloro-1H-indol-2-yl)pyrazine-2-carboxamide;
N-((4-aminotetrahydro-2H-pyran-4-yl)methyl)-6-(3-methyl-1H-indol-2-yl)pyrazine-2-carboxamide;
Methyl 2-(6-((2-amino-2-methylpropyl)carbamoyl)pyrazin-2-yl)-1H-indole-6-carboxylate;
N-((1r,4r)-4-aminocyclohexyl)-6-(3-methyl-5-(trifluoromethoxy)-1H-indol-2-yl)pyrazine-2-carboxamide;
Methyl 2-(6-((2-amino-2-methylpropyl)carbamoyl)pyrazin-2-yl)-1H-indole-5-carboxylate;
N-(2-amino-2-methylpropyl)-6-(3-(trifluoromethyl)-1H-indol-2-yl)pyrazine-2-carboxamide;
N-(3-aminopropyl)-6-(3-methyl-1H-indol-2-yl)pyrazine-2-carboxamide;
N-(azetidin-3-yl)-6-(3-methyl-1H-indol-2-yl)pyrazine-2-carboxamide;
N-(2-amino-2-methylpropyl)-6-(4-methoxy-1H-indol-2-yl)pyrazine-2-carboxamide;
N-(2-amino-2-methylpropyl)-6-(6-methoxy-1H-indol-2-yl)pyrazine-2-carboxamide;
N-(3-aminocyclopentyl)-6-(3-methyl-1H-indol-2-yl)pyrazine-2-carboxamide;
N-(2-amino-2-methylpropyl)-6-(4,5,6,7-tetrahydro-1H-indol-2-yl)pyrazine-2-carboxamide;
N-(2-amino-2-methylpropyl)-6-(5-methoxy-1H-indol-2-yl)pyrazine-2-carboxamide;
N-(2-amino-2-methylpropyl)-6-(6-chloro-5-fluoro-1H-pyrrolo[2,3-b]pyridin-2-yl)pyrazine-2-carboxamide;
N-(2-amino-2-methylpropyl)-N-methyl-6-(3-methyl-1H-indol-2-yl)pyrazine-2-carboxamide;
N-(2-amino-2-methylpropyl)-6-(6-cyano-1H-indol-2-yl)pyrazine-2-carboxamide;
6-(3-methyl-1H-indol-2-yl)-N-(piperidin-4-yl)pyrazine-2-carboxamide;
N-(2-amino-3-hydroxypropyl)-6-(3-methyl-1H-indol-2-yl)pyrazine-2-carboxamide;
N-((3-aminooxetan-3-yl)methyl)-6-(3-methyl-1H-indol-2-yl)pyrazine-2-carboxamide;
N-(2-amino-2-methylpropyl)-6-(1H-indol-2-yl)pyrazine-2-carboxamide;
N-(2-amino-2-methylpropyl)-6-(5-cyano-1H-indol-2-yl)pyrazine-2-carboxamide;
6-(3-Methyl-1H-indol-2-yl)-N-(2-methyl-2-morpholinopropyl)pyrazine-2-carboxamide;
N-(2-amino-2-methylpropyl)-6-(5-fluoro-6-methoxy-1H-indol-2-yl)pyrazine-2-carboxamide;
N-((1r,4r)-4-aminocyclohexyl)-6-(3-methyl-1H-indol-2-yl)pyrazine-2-carboxamide;
N-(2-amino-2-methylpropyl)-6-(6-chloro-1H-pyrrolo[2,3-b]pyridin-2-yl)pyrazine-2-carboxamide;
N-((1r,4r)-4-aminocyclohexyl)-6-(5-(trifluoromethoxy)-1H-indol-2-yl)pyrazine-2-carboxamide;
N-(2-amino-2-methylpropyl)-6-(5,7-dichloro-1H-pyrrolo[2,3-c]pyridin-2-yl)pyrazine-2-carboxamide;
N-(2-amino-2-methylpropyl)-6-(5-chloro-1H-pyrrolo[2,3-c]pyridin-2-yl)pyrazine-2-carboxamide;
N-(2-amino-2-methylpropyl)-6-(6-chloro-1H-pyrrolo[3,2-c]pyridin-2-yl)pyrazine-2-carboxamide;
N-(2-amino-2-methylpropyl)-6-(3,6,6-trimethyl-4-oxo-4,5,6,7-tetrahydro-1H-indol-2-yl)pyrazine-2-carboxamide;
N-(2-amino-2-methylpropyl)-6-(4-chloro-1H-pyrrolo[3,2-c]pyridin-2-yl)pyrazine-2-carboxamide;
(rac)-N-((1r,2s)-2-aminocyclohexyl)-6-(3-methyl-1H-indol-2-yl)pyrazine-2-carboxamide;
(rac)-N-((3r,4s)-4-aminotetrahydrofuran-3-yl)-6-(3-methyl-1H-indol-2-yl)pyrazine-2-carboxamide;
N-(2-amino-2-methylpropyl)-6-(3-cyano-1H-indol-2-yl)pyrazine-2-carboxamide;
N-(2-amino-2-methylpropyl)-6-(3-methyl-5-(2-oxopyrrolidin-1-yl)-1H-indol-2-yl)pyrazine-2-carboxamide;
N-(2-amino-2-methylpropyl)-6-(3-methyl-5-(methylsulfonyl)-1H-indol-2-yl)pyrazine-2-carboxamide;
N-(2-hydroxy-2-methylpropyl)-6-(3-methyl-1H-indol-2-yl)pyrazine-2-carboxamide;
(rac)-N-((1r,2r)-2-aminocyclohexyl)-6-(3-methyl-1H-indol-2-yl)pyrazine-2-carboxamide;
6-(3-methyl-1H-indol-2-yl)-N-neopentylpyrazine-2-carboxamide;
N-(2-amino-2-methylpropyl)-6-(7-chloro-1H-pyrrolo[2,3-c]pyridin-2-yl)pyrazine-2-carboxamide;
N-(2-amino-2-methylpropyl)-6-(1H-pyrrolo[2,3-c]pyridin-2-yl)pyrazine-2-carboxamide;
5-(6-((2-amino-2-methylpropyl)carbamoyl)pyrazin-2-yl)-4H-thieno[3,2-b]pyrrole-2-carboxamide;
N-(2-amino-2-methylpropyl)-6-(5-(aminomethyl)-3-methyl-1H-indol-2-yl)pyrazine-2-carboxamide;
N-(2-amino-2-methylpropyl)-6-(5-phenyl-1H-pyrrol-2-yl)pyrazine-2-carboxamide;
N-(2-amino-2-methylpropyl)-6-(5-fluoro-6-hydroxy-1H-indol-2-yl)pyrazine-2-carboxamide;
N-(2-amino-2-methylpropyl)-6-(7-methoxy-1H-indol-2-yl)pyrazine-2-carboxamide;
5-(6-((2-amino-2-methylpropyl)carbamoyl)pyrazin-2-yl)-N,N-dimethyl-4H-thieno[3,2-b]pyrrole-2-carboxamide;
   and pharmaceutically acceptable salts thereof.

Embodiment 30. A compound according to any of the embodiments herein of formula (IIa), or a pharmaceutically acceptable salt thereof: (IIa).

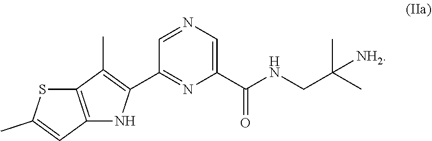 [SMILES: Cc1cc2[nH]c(-c3cncc(C(=O)NCC(C)(C)N)n3)c(C)c2s1]

Embodiment 31. A compound according to any of the embodiments herein of formula (IIb), or a pharmaceutically acceptable salt thereof:

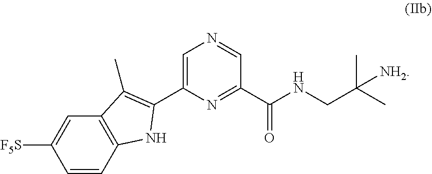

Embodiment 32. A compound according to any of the embodiments herein of formula (IIc), or a pharmaceutically acceptable salt thereof: (IIc).

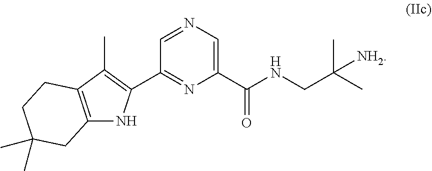 [SMILES: Cc1c(-c2cncc(C(=O)NCC(C)(C)N)n2)[nH]c2c1CCC(C)(C)C2]

Embodiment 33. A compound or pharmaceutically acceptable salt thereof according to any of the embodiments herein, for use as a medicament.

Embodiment 34. A compound or pharmaceutically acceptable salt thereof according to any of the embodiments herein, for use in treating a Plasmodium related disease.

Embodiment 35. Use of a compound or pharmaceutically acceptable salt thereof according to any of the embodiments herein, in the manufacture of a medicament for treating a Plasmodium related disease.

Embodiment 36. A method of treating a Plasmodium related disease, the method comprising administering to a subject in need thereof, a therapeutically effective amount of a compound according to any of the embodiments herein.

Embodiment 37. The compound for use according to any of the embodiments herein, the use according to any of the embodiments herein, wherein the Plasmodium related disease is malaria.

Embodiment 38. The compound for use according to any of the embodiments herein, the use according to any of the embodiments herein, or the method according to any of the embodiments herein, wherein the compound according to any of the embodiments herein is administered in combination with one or more therapeutically active agent(s).

Embodiment 39. The compound for use according to any of the embodiments herein, the use according to any of the embodiments herein, or the method according to any of the embodiments herein, wherein the compound according to any of the embodiments herein is administered prior to, simultaneously with, or after the therapeutically active agent.

Embodiment 40. The compound for use, the use, or the method according to any of the embodiments herein, wherein the therapeutically active agent is selected from a kinase inhibitor, an anti-malarial drug and an anti-inflammatory agent.

Embodiment 41. The compound for use, the use according, or the method according to any of the embodiments herein, wherein the active agent is an anti-malarial drug selected from proguanil, chlorproguanil, trimethoprim, chloroquine, mefloquine, lumefantrine, atovaquone, pyrimethamine-sulfadoxine, pyrimethamine-dapsone, halofantrine, quinine, quinidine, amodiaquine, amopyroquine, sulphonamides, artemisinin, arteflene, artemether, artesunate, primaquine, pyronaridine, KAE-609, KAF-156 and INE963.

Embodiment 42. The method according to any of the embodiments herein, wherein the subject is human.

Embodiment 43. A pharmaceutical composition comprising the compound according to any of the embodiments herein, and one or more pharmaceutically acceptable carriers.

It is understood that any of elements listed in the above numbered embodiments can be individually selected and/or combined with one another, as other embodiments of the invention.

### Definitions

For the purpose of interpreting this specification, the following definitions will apply unless specified otherwise and when appropriate, terms used in the singular will also include the plural and vice versa. It must be noted that as used herein and in the appended claims, the singular forms “a”, “an” and “the” include the plural unless the context clearly dictates otherwise.

Thus, for example, reference to “the compound” includes reference to one or more compounds, and so forth.

As used herein, the term “substituent” refers to a radical group which replaces a hydrogen atom in a given molecule.

As used herein, the term “alkyl” refers to a straight or branched hydrocarbon chain radical consisting solely of carbon and hydrogen atoms, containing no unsaturation, and which is attached to the rest of the molecule by a single bond. For instance, C₁-C₃ alkyl contains from 1 to 3 carbon atoms. Examples of C₁-C₃-alkyl include, methyl (Me), ethyl (Et), n-propyl and 1-methylethyl (iso-propyl).

As used herein, the term “halogen”, “halo”, “hal”, etc. refers to fluorine, chlorine, bromine or iodine. Halogen-substituted groups and moieties, such as alkyl substituted by halogen (haloalkyl) can be mono-, poly- or per-halogenated.

As used herein, the term “haloalkyl” refers to an alkyl radical as defined herein, wherein one or more of the hydrogen atoms of said alkyl has been replaced with a halogen atom. In some embodiments said one or more halogen atom(s) are each fluorine atom(s), in which case the “haloalkyl” is a “fluoroalkyl”.

As used herein, the term “alkylene” refers to a straight-chain or branched divalent radical of an alkyl group. For instance, “C₁-C₅alkylene” contains from 1 to 5 carbon atoms e.g., —CH₂—, —CH₂CH₂—, —CH₂CH₂CH₂—, —CH(CH₃)₂—, —CH(CH₃)CH₂CH₂CH₂— etc.

As used herein, the term “cycloalkyl” refers to a saturated carbocyclic ring radical. C₃-C₆Cycloalkyl is any such ring radical containing 4 to 6 carbon atoms i.e. cyclobutyl, cyclopentyl and cyclohexyl. C₃cycloalkyl is a ring radical containing 3 carbon atoms i.e. cyclopropyl. The cycloalkyl can be a monocyclic or a polycyclic ring, including a fused or bridged bicyclic ring system (e.g.

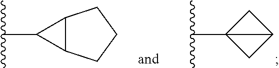 [SMILES: *C1C2CCCC12 ; *C12CC1C2]

respectively). In some embodiments, however, the cycloalkyl is a monocyclic ring. As used herein, the term “cycloalkylene” refers to a divalent radical of a cycloalkyl group, e.g.

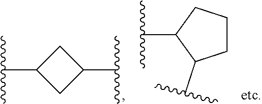

As used herein, the term “cyclohaloalkyl” refers to a saturated carbocyclic ring radical, wherein one or more of the hydrogen atoms of said alkyl has been replaced with a halogen atom (e.g. fluorine, chlorine). As used herein, the term “cyclohaloalkylene” refers to a divalent radical of a cyclohaloalkyl, e.g.

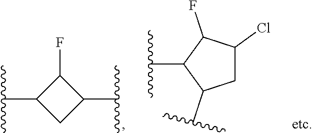

The cyclohaloalkyl can be a monocyclic or a polycyclic ring, including a fused or bridged bicyclic ring system. In some embodiments, the cyclohaloalkyl is a monocyclic ring.

As used herein, the term “heterocyclyl”, “heterocycle”, “heterocyclic” etc. refers to a heterocyclic radical that is saturated or partially unsaturated (in some embodiments saturated) but not aromatic, and can be a monocyclic or a polycyclic ring, including a fused or bridged bicyclic ring system. In some embodiments, the heterocyclyl is a monocyclic ring. In some embodiments, the heterocyclyl is saturated. A heterocyclyl contains at least one non-carbon atom as a ring member, typically N, O or S unless otherwise specified, the remaining ring atoms therefore being carbon. Where a (n unsubstituted) heterocyclyl contains S as a heteroatom, the S can be in the form of S, SO or SO₂. In some embodiments, the heteroatom is O or N. The term “4-6 membered heterocyclyl comprising one heteroatom selected from O and N” refers to a ring radical containing 4 to 6 ring atoms comprising 1 heteroatom (either O or N), with the remaining ring atoms being carbon. As used herein, the term “heterocyclylene” refers to refers to a divalent radical of a “heterocyclyl”. For instance, a 4-6 membered heterocyclylene comprising one heteroatom selected from O and N includes

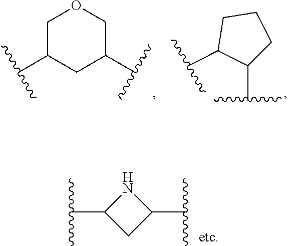

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diastereomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto. When both a basic group and an acid group are present in the same molecule, the compounds of the present invention may also form internal salts, e.g., zwitterionic molecules.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

### DESCRIPTION OF THE EMBODIMENTS

The invention provides a novel class of compounds, pharmaceutical compositions comprising such compounds and methods of using such compounds to treat or prevent diseases or disorders associated with a parasite. In particular, the compounds can be used to treat malaria.

In some embodiments, with reference to compounds of Formula (I), R¹ is H or CH₃. In an embodiment, R¹ is H.

In some embodiments, with reference to compounds of Formula (I), R² is selected from C₁-C₃alkyl, Cl, hydrogen, C₁fluoroalkyl (e.g. CF₃) and cyano. In an embodiment, R² is CH₃.

In some embodiments, with reference to compounds of Formula I, the moiety:

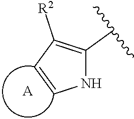

is

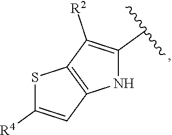

e.g. selected from the group consisting of:

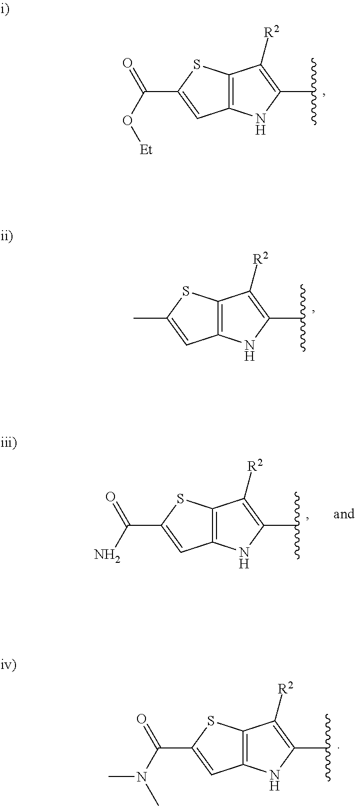

In some embodiments, with reference to compounds of Formula I, the moiety:

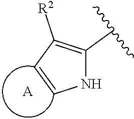

is

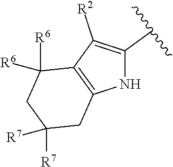

e.g. selected from the group consisting of:

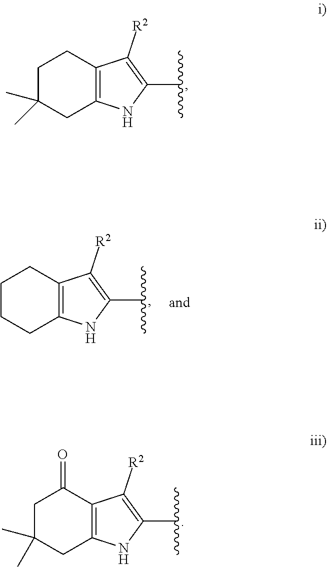

In some embodiments, the moiety:

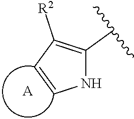

is

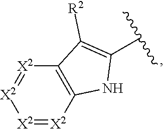

wherein each X² is independently selected from the group consisting of N and CR³, with the proviso that at least one X² is CR³. In some embodiments, at least two X² are each CR³. In some embodiments, at least three X² are each CR³. In some embodiments, all four X² are each CR³.

In some embodiments, the moiety:

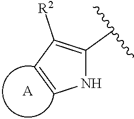

is selected from the group consisting of:

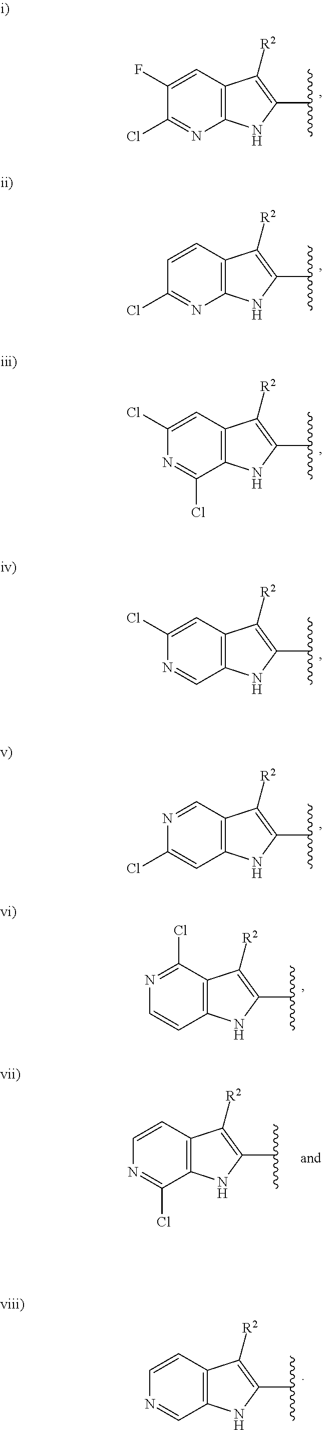

In some embodiments, the moiety:

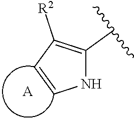

is

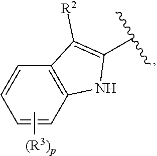

p is 0, 1, 2, 3 or 4, and wherein each R³ is independently selected from the group consisting of halo, SF₅, methyl, hydroxyl, cyano, OMe, SO₂Me, C(O)OMe, O—C₁haloalkyl, C₁haloalkyl, CH₂NH₂, OCH₂C₆H₅, C(O)—N(H)—C₁-C₄alkylene-NH₂ and

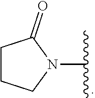 [SMILES: *N1CCCC1=O]

In some embodiments, p is 0, 1, 2 or 3. In some embodiments, p is 0, 1 or 2. In some embodiments, p is 1 or 2. In some embodiments, each R³ is independently selected from the group consisting of halo, OC₁haloalkyl, SF₅, methyl and C(O)OMe.

In some embodiments, the moiety:

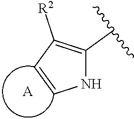

is

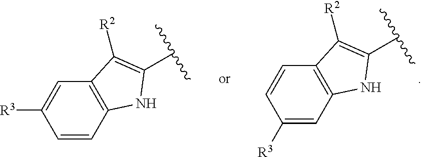

In some embodiments, the moiety:

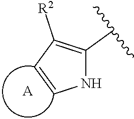

is

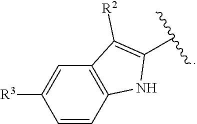

In some embodiments, R³ is halo, OCF₃, SF₅, or OCHF₂. In some embodiments, R³ is SF₅.

In some embodiments, the moiety:

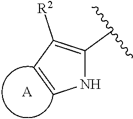

is selected from the group consisting of:

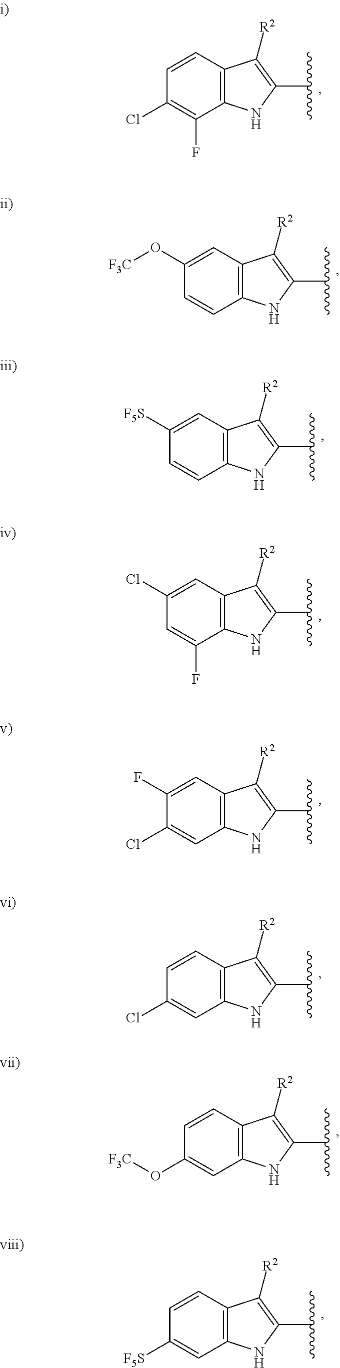

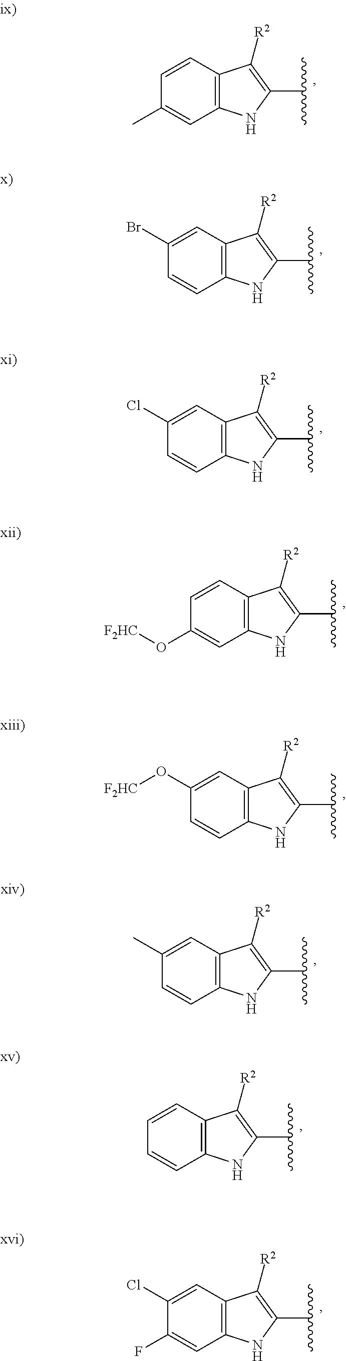

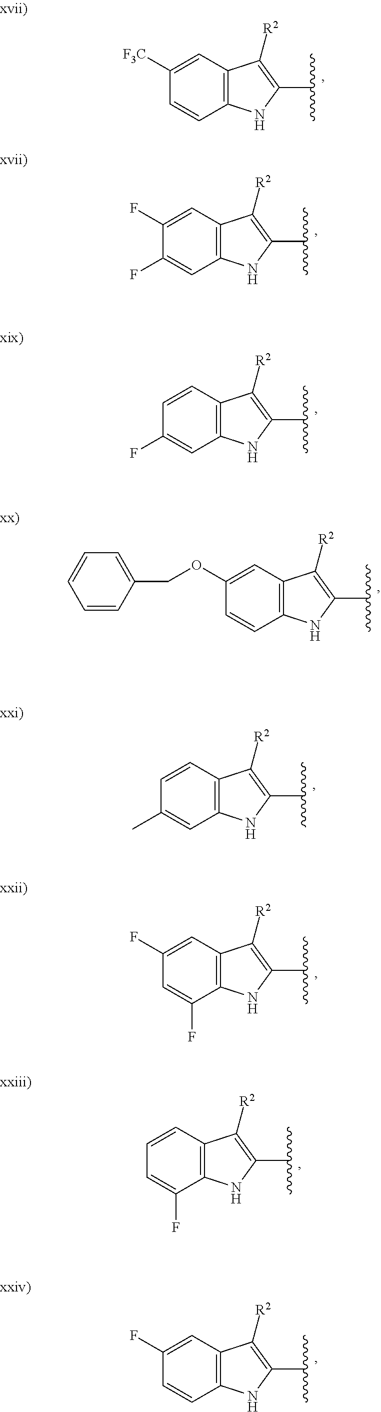

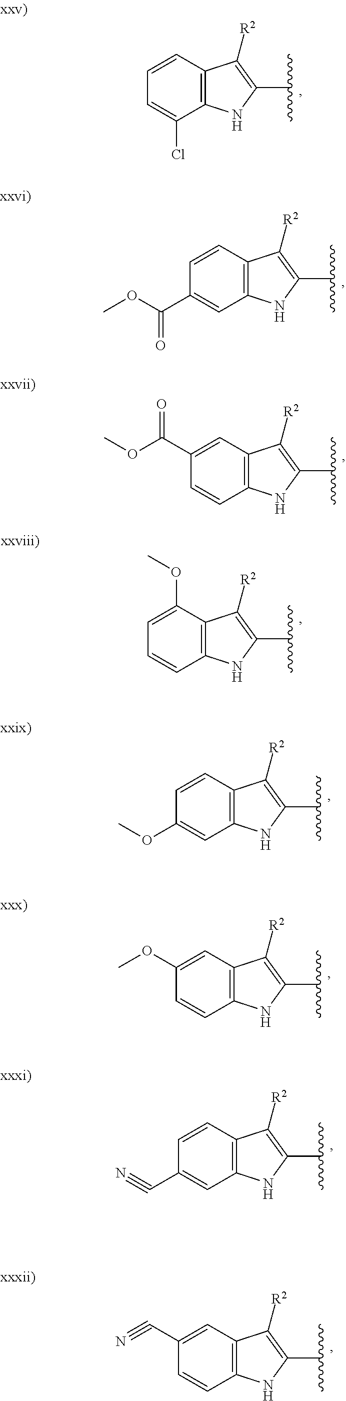

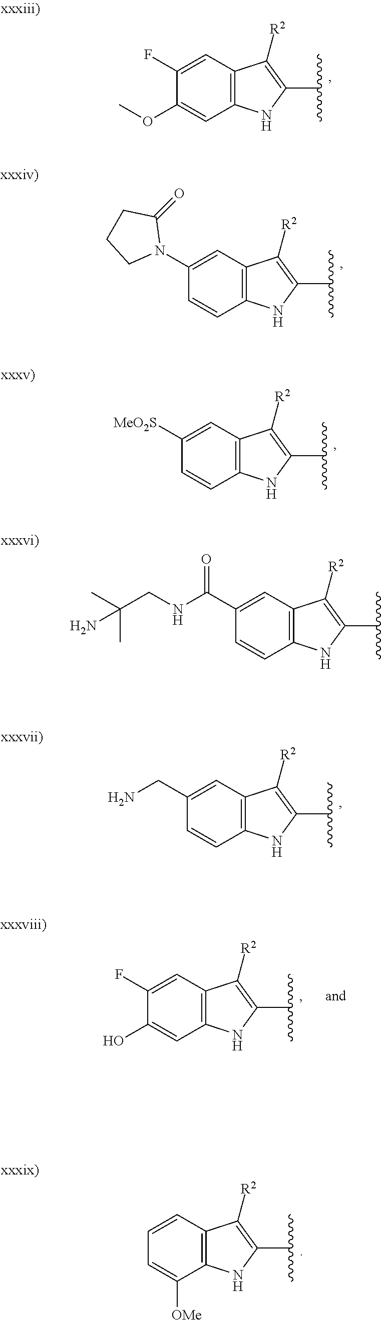

In some embodiments, the moiety:

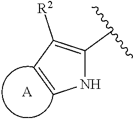

is

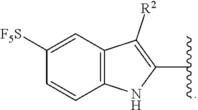

In some embodiments, L¹ is C₁-C₅alkylene, optionally substituted with OH or C₃cycloalkyl. In an embodiment, L¹ is unsubstituted C₁-C₅alkylene. In an embodiment, L¹ is unsubstituted C₄alkylene.

In some embodiments, X¹ is:
i) NH₂,
ii) a) C₃-C₆cycloalkyl substituted with NH₂, b) C₃-C₆cycloalkyl (in some embodiments C₃-C₆cyclofluoroalkyl) substituted with NH₂ or c) 4-6 membered heterocyclyl comprising one heteroatom selected from O and N, said 4-6 membered heterocyclyl being substituted with NH₂, or

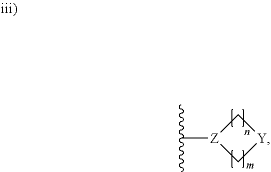

wherein Z is CH, Y is NH, n is 1 or 2 and m is 1 or 2 (in some embodiments, n and m are each 1 or n and m are each 2).

In some embodiments, X¹ is NH₂.

In some embodiments, X¹ is:

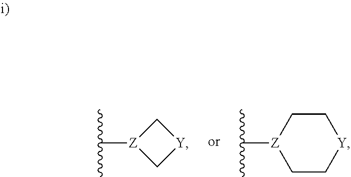

wherein Z is CH, Y is NH.

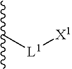

In some embodiments,

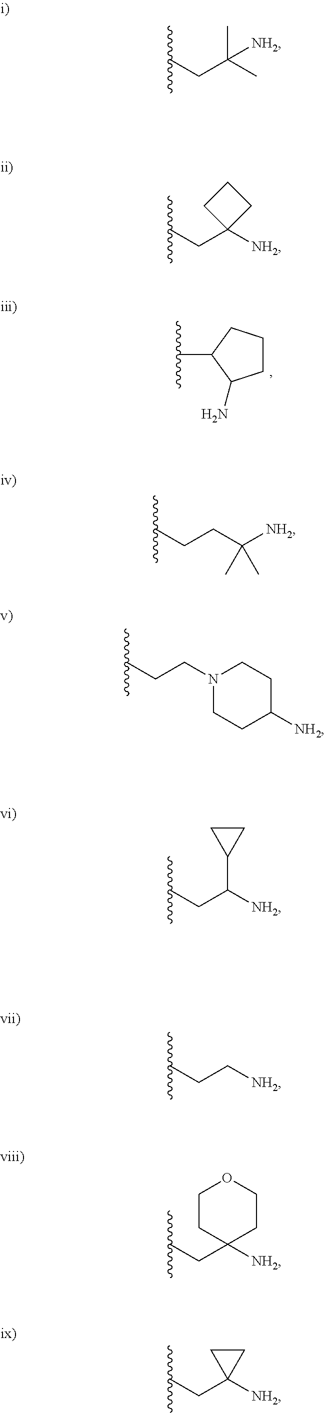

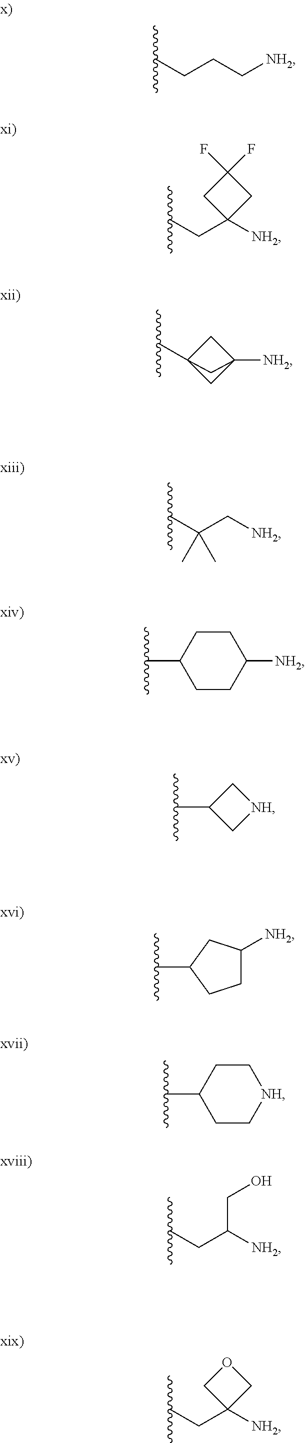

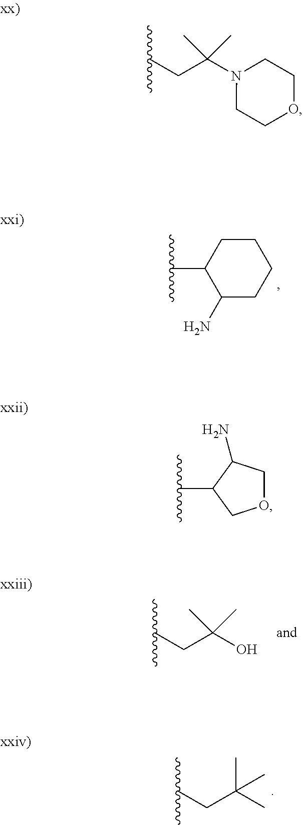

is selected from:

In some embodiments, the moiety

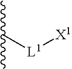

is

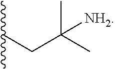 [SMILES: *CC(C)(C)N]

In further embodiments are compounds selected from any one of Examples 1 to 110 or a pharmaceutically acceptable salt thereof.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present invention provides compounds according to any one of embodiments 1 to 44+ in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

In another aspect, the present invention provides compounds according to any one of embodiments 1 to 44+, in sodium, potassium, ammonium, calcium, magnesium, iron, silver, zinc, copper, isopropylamine, benzathine, cholinate, diethanolamine, diethylamine, lysine, meglumine, piperazine or tromethamine salt form.

Any formula given herein is also intended to represent unlabelled forms as well as isotopically labelled forms of the compounds. Isotopically labelled compounds have structures depicted by the formulae given herein except that one or more atoms are replaced by an atom having a selected atomic mass or mass number. Isotopes that can be incorporated into compounds of the invention include, for example, isotopes of hydrogen.

Further, incorporation of certain isotopes, particularly deuterium (i.e., 2H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, phosphorous, fluorine, and chlorine, such as ³H, ¹¹C, ¹³C, ¹⁴C, ¹⁵N, ¹⁸F ³¹P, ³²P, ³⁵S, ³⁶Cl, ¹²³I, ¹²⁴I, ¹²⁵I respectively. Accordingly, it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

### Pharmaceutical Composition

As used herein, the term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to at least partially alleviate, inhibit, prevent and/or ameliorate a Plasmodium related disease (e.g. malaria).

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically, or in quality of life from such treatment.

As used herein, the term “a”, “an”, “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O, O′-p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

### Methods of Synthesizing the Compounds of the Invention

All methods described herein can be performed in any suitable order unless otherwise indicated herein or otherwise clearly contradicted by context. The use of any and all examples, or exemplary language (e.g. “such as”) provided herein is intended merely to better illustrate the invention and does not pose a limitation on the scope of the invention otherwise claimed.

The compounds of the present application can be prepared by those skilled in the art of organic synthesis using commercially available starting materials, compounds known in the literature, or from readily prepared intermediates, by employing standard synthetic methods and procedures either known to those skilled in the art, or which will be apparent to the skilled chemist in light of the teachings herein.

The compounds of Formula (I) may be prepared by methods as set forth in the following synthetic reaction schemes. In the schemes described below, it is well understood that protecting groups for sensitive or reactive groups are employed where necessary in accordance with general principles of chemistry. Protecting groups are manipulated according to standard methods of organic synthesis as described for example in Protective Groups in Organic Synthesis, 3rd edition, John Wiley & Sons: New York, 1999 or Protecting Groups, 3rd edition, Thieme, Stuttgart, 2004. Protective groups are removed at a convenient stage of the compound synthesis using methods that are readily apparent to those skilled in the art.

Those skilled in the art will recognize if a stereocenter exists in the compounds disclosed herein. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

Compounds of the present disclosure can be synthesized by following the steps outlined in Scheme I. Starting materials are either commercially available or made by known procedures in the reported literature or as illustrated.

In the following general methods, R¹, L¹, X¹ and Ring A are as previously defined in the above embodiments, or limited to designations in the Schemes. Unless otherwise stated, starting materials are either commercially available or are prepared by known methods.

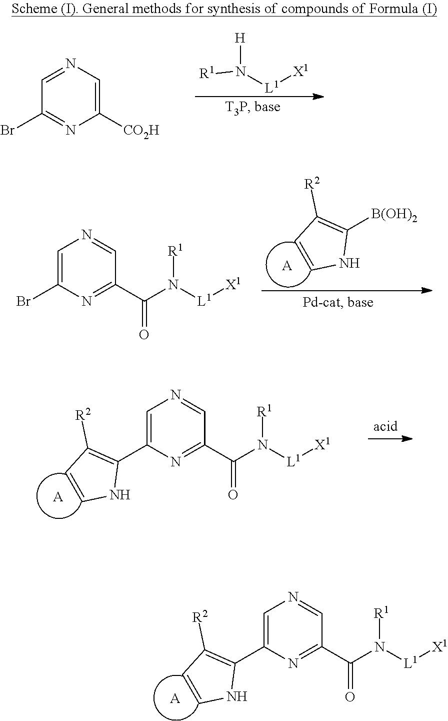

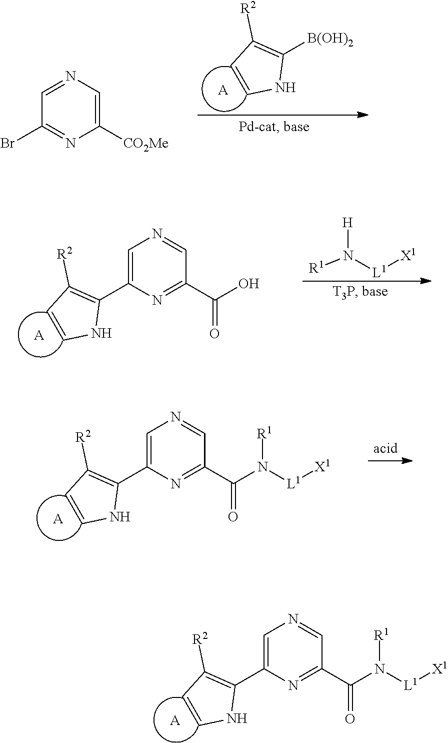

Scheme I shows two alternative general methods for synthesizing compounds of Formula (I). Alternative 1 begins with coupling of a 2-halo-pyrazine-6-carboxylic acid under standard amide forming conditions with an appropriately substituted amine. The amides are then further reacted with appropriately substituted boronic acid or esters under typical Suzuki-coupling conditions. Alternative 2 starts with a transition metal catalyzed coupling of an appropriately substituted boronic acid or ester with a 2-halo-pyrazine-6-carboxylic ester with concomitant hydrolysis of the ester to the carboxylic acid. The resulting acids can be reacted under standard amide forming conditions with an appropriately substituted amine. Finally, the resulting amines from either alternative can be deprotected using typical conditions for an N-Boc group such trifluoro acetic acid or formic acid.

Detailed descriptions of the synthesis of compounds of the Invention are given in the Examples, infra.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material. Compounds of the invention and intermediates can also be converted into each other according to methods generally known to those skilled in the art.

In another aspect, the present invention provides a pharmaceutical composition comprising a compound of the present invention, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art.

Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) for tablets, also binders, e.g., magnesium aluminum silicate, starch paste, gelatin, tragacanth, methylcellulose, sodium carboxymethylcellulose and/or polyvinylpyrrolidone; if desired;
d) disintegrants, e.g., starches, agar, alginic acid or its sodium salt, or effervescent mixtures; and
e) absorbents, colorants, flavors and sweeteners.

### METHODS OF USING THE INVENTION

The compounds of formulae (I), (IIa), (IIb), (IIc) and (IId) in free form or in pharmaceutically acceptable salt form, exhibit valuable pharmacological properties, for example in the treatment of Plasmodium related diseases (for example, malaria), for example as indicated in in vitro tests as provided in the next sections, and are therefore indicated for therapy or for use as research chemicals, e.g. as tool compounds.

Compounds of the invention may have activity against protein kinases, but are relatively inactive against certain protein kinases, e.g. ataxia telangiectasia mutated (ATM) and Rad3 related (together, ATR) kinase.

Compounds of the invention are useful in the treatment and/or prevention of infections such as those caused by Plasmodium falciparum; Plasmodium vivax; Plasmodium ovale; and Plasmodium malaria, Trypanosoma cruzi and parasites of the Leishmania genus, such as, for example, Leishmania donovani.

Malaria is an infectious disease caused by four protozoan parasites: Plasmodium falciparum; Plasmodium vivax; Plasmodium ovale; and Plasmodium malaria. These four parasites are typically transmitted by the bite of an infected female Anopheles mosquito. Malaria is a problem in many parts of the world and over the last few decades the malaria burden has steadily increased. An estimated 1-3 million people die every year from malaria-mostly children under the age of 5. This increase in malaria mortality is due in part to the fact that Plasmodium falciparum, the deadliest malaria parasite, has acquired resistance against nearly all available antimalarial drugs, even resistance to artemisinins is emerging.

The phylum, Apicomplexa, contains many members that are human or animal pathogens including, but not limited to, Plasmodium spp. (Malaria), Toxoplasma gondii (congenital neurological defects in humans), Eimeria spp. (poultry and cattle pathogens), Cryptosporidia (opportunistic human and animal pathogens), Babesia (cattle parasites) and Theileria (cattle parasites). The pathogenesis associated with these parasitic diseases is due to repeated cycles of host-cell invasion, intracellular replication and host-cell lysis. Therefore, understanding parasite proliferation is essential for development of novel drugs and vaccines, for example, to treat malaria.

In vertebrate hosts, the parasite undergoes two main phases of development, the hepathocytic and erythrocytic phases, but it is the erythrocytic phase of its life cycle that causes severe pathology. During the erythrocytic phase, the parasite goes through a complex but well synchronized series of stages, suggesting the existence of tightly regulated signaling pathways.

In accordance with the foregoing, the present invention further provides a method for preventing or treating malaria in a subject in need of such treatment, which method comprises administering to said subject a therapeutically effective amount of a compound of Formula (I), Formula (IIa), Formula (IIb), Formula (IIc), Formula (IId), a compound of the Examples or a pharmaceutically acceptable salt thereof. The required dosage will vary depending on the mode of administration, the particular condition to be treated and the effect desired.

The pharmaceutical composition or combination of the present invention may, for example, be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

### Combination Product and Combination Therapy of the Invention

“Combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow for the combination partners to have a cooperative, e.g. synergistic effect. The single components may be packaged in a kit or separately. One or both of the components (e.g. powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow for the combination partners to have a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g. tablets, capsules, powders, and liquids) for each active ingredient. Powders and/or liquids may be reconstituted or diluted to a desired dose prior to administration.

In addition, such administration also encompasses use of each type of therapeutic agent in a sequential manner, either at approximately the same time or at different times. In either case, the treatment regimen will provide beneficial effects of the drug combination in treating the conditions or disorders described herein.

In a further embodiment, the second agent is selected from a kinase inhibitor, an anti-malarial drug and an anti-inflammatory agent. The anti-malarial drug is selected from proguanil, chlorproguanil, trimethoprim, chloroquine, mefloquine, lumefantrine, atovaquone, pyrimethamine-sulfadoxine, pyrimethamine-dapsone, halofantrine, quinine, quinidine, amodiaquine, amopyroquine, sulphonamides, artemisinin, arteflene, artemether, artesunate, primaquine, pyronaridine, KAE-609, KAF-156 and INE963.

The compounds of the present invention may be administered either simultaneously with, or before, or after, one or more other therapeutic agent. The compounds of the present invention may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the invention.

Thus, in another aspect, the invention provides a combination, in particular a pharmaceutical combination, comprising (e.g. a therapeutically effective amount of) a compound of any one of formulae (I), (IIa), (IIb), (IIc) and (IId) (in particular according to any one of embodiments 1 to 44+), or a pharmaceutically acceptable salt thereof, and one or more other therapeutically active agents.

In one embodiment, the invention provides a product comprising a compound of any one of formulae (I), (IIa), (IIb), (IIc) and (IId), in particular according to any one of embodiments 1 to 44+), or a pharmaceutically acceptable salt thereof, and at least one other therapeutic agent as a combined preparation for simultaneous, separate or sequential use in therapy.

In one embodiment, the invention provides a pharmaceutical combination comprising a compound of any one of formulae (I), (IIa), (IIb), (IIc) and (IId), (in particular according to any one of embodiments 1 to 44+), or a pharmaceutically acceptable salt thereof, and another therapeutic agent(s). Optionally, the pharmaceutical combination may comprise a pharmaceutically acceptable carrier, as described above

In one embodiment, the invention provides a kit comprising two or more separate pharmaceutical compositions, at least one of which contains a compound of any one of formulae (I), (IIa), (IIb), (IIc) and (IId), (in particular according to any one of embodiments 1 to 44+), or a pharmaceutically acceptable salt thereof. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

### EXAMPLES

The disclosure is further illustrated by the following examples and synthetic methods, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

The compounds of the present invention can be produced by organic synthesis methods known to one of ordinary skill in the art as shown in the following examples. All starting materials, building blocks, reagents, acids, bases, dehydrating agents, solvents, and catalysts utilized to synthesize the compounds of the present invention are either commercially available or can be produced by organic synthesis methods known to one of ordinary skill in the art. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (2014) Protective Groups in Organic Synthesis, 5th edition, John Wiley & Sons). These groups are removed at a convenient stage of the compound synthesis using methods that are readily apparent to those skilled in the art. Unless otherwise noted, reagents and solvents were used as received from commercial suppliers.

The chemical names were generated using ChemDraw Professional v17.1.0.105 from PerkinElmer.

Temperatures are given in degrees Celsius. As used herein, unless specified otherwise, the term “room temperature” or “ambient temperature” means a temperature of from 15° C. to 30° C., such as from 20° C. to 30° C., such as from 20° C. to 25° C. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

### ABBREVIATIONS

Abbreviations used in the following examples and elsewhere herein are:

| | |
|---|---|
| AcOH | Acetic acid |
| B₂Pin₂ | Bis(pinacolato)diboron |
| br | Broad signal |
| B(OⁱPr)₃ | Triisopropyl borate |
| (Boc)₂O | Di-tert-butyl dicarbonate |
| d | Doublet |
| CH₂Cl₂ | Dichloromethane |
| i-Pr₂NEt | Diisopropylethylamine |
| DMAP | 4-Dimethylaminopyridine |
| DMF | Dimethyl formamide |
| DMSO | Dimethylsulfoxide |
| dtbpy | Di-tert-butyl-2.2′-dipyridyl |
| eq. | Equivalent(s) |
| Et₃N | Triethylamine |
| Et₂O | Diethyl ether |
| EtOAc | Ethyl acetate |
| EtOH | Ethanol |
| h | Hour |
| H₂O | Water |
| HATU | 1-[Bis(dimethylamino)methylene]-1H-1,2,3-triazolo[4,5-b]pyridinium |
| | 3-oxid hexafluorophosphate |
| i-PrOH | 2-Propanol |
| [Ir(cod)OMe]₂ | (1,5-Cyclooctadiene)(methoxy)iridium(I) dimer |
| KHSO₄ | Potassium bisulfate |
| K₂CO₃ | Potassium carbonate |
| K₃PO₄ | Potassium phosphate tribasic |
| KOAc | Potassium acetate |
| KOH | Potassium hydroxide |
| KO^{t}Bu | Potassium tert-butoxide |
| LDA | Lithium diisopropylamide |
| LiAlH₄ | Lithium aluminium hydride |
| LiBH₄ | Lithium borohydride |
| LiOH | Lithium hydroxide |
| m | Multiplet |
| CH₃CN | Acetonitrile |
| MeI | Methyl iodide |
| MeOH | Methanol |
| MS | Mass spectrometry |
| MsCl | Methanesulfonyl chloride |
| N | Normality |
| N₂ | Nitrogen |
| n-BuLi | n-Butyllithium |
| NaH | Sodium hydride |
| NaHCO₃ | Sodium bicarbonate |
| Na₂SO₄ | Sodium Sulphate |
| NCS | N-Chlorosuccinimide |
| NH₄Cl | Ammonium Chloride |
| NMR | Nuclear magnetic resonance spectrometry |
| Pd/C | Palladium on carbon |
| PdCl₂(Ph₃P)₂ | Bis(triphenylphosphine)palladium(II) dichloride |
| Pd(dppf)Cl₂ | 1,1′-Bis(diphenylphosphino)ferrocene] palladium (II) dichloride |
| Ph₃P | Triphenyl phosphine |
| q | Quartet |
| s | Singlet |
| t | Triplet |
| t-BuOH | tert-Butyl alcohol |
| T₃P | 1-Propanephosphonic anhydride |
| TFA | Trifluoroacetic acid |
| THF | Tetrahydrofuran |

### SYNTHESIS OF THE INTERMEDIATES

### Intermediate A1: tert-Butyl (1-(6-bromopyrazine-2-carboxamido)-2-methylpropan-2-yl)carbamate

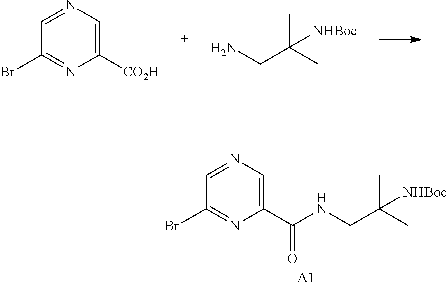

To a solution of 6-bromopyrazine-2-carboxylic acid (1.649 g, 8.12 mmol) and tert-butyl (1-amino-2-methylpropan-2-yl) carbamate (1.606 g, 8.53 mmol) in DMF (10 mL) was added Et₃N (1.25 mL, 8.94 mml) followed by T₃P (5.32 mL, 8.94 mmol, 50% w/w in EtOAc) under cooling in ice-bath. The reaction mixture was stirred at rt for 40 min. After dilution with EtOAc, the mixture was washed with aq. KHSO₄, water, sat. aq. NaHCO₃, water, and brine, dried over Na₂SO₄, filtered, and concentrated in vacuo. The residue was purified by flash chromatography over silica gel (EtOAc/heptane gradient 0 to 50%) to afford the title compound (2.72 g, 90% yield). ¹H NMR (500 MHZ, DMSO-d₆) δ 9.16 (d, J=0.5 Hz, 1H), 9.09 (d, J=0.5 Hz, 1H), 8.77 (t, J=6.7 Hz, 1H), 3.42 (d, J=6.6 Hz, 2H), 1.39 (s, 9H), 1.21 (s, 6H). LCMS (ESI) m/z calcd for C₁₄H₂₁⁸¹BrN₄O₃ 374.1, found 375.1 (M+H)⁺.

Intermediates A2-A10 were prepared in a manner analogous to Intermediate A1.

| Intermediate | Structure |
|---|---|
| A2 | 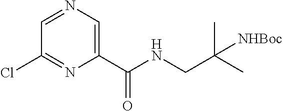 [SMILES: CC(C)(CNC(=O)c1cncc(Cl)n1)NC(=O)OC(C)(C)C] |
| A3 | 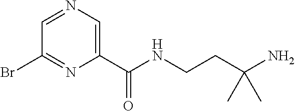 [SMILES: CC(C)(N)CCNC(=O)c1cncc(Br)n1] |
| A4 | 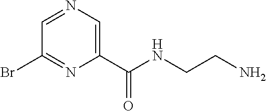 [SMILES: NCCNC(=O)c1cncc(Br)n1] |
| A5 | 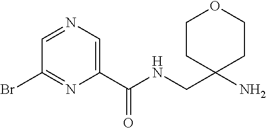 [SMILES: NC1(CNC(=O)c2cncc(Br)n2)CCOCC1] |
| A6 | 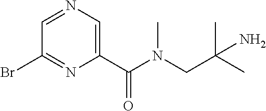 [SMILES: CN(CC(C)(C)N)C(=O)c1cncc(Br)n1] |
| A7 | 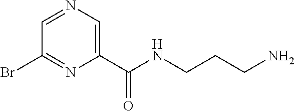 [SMILES: NCCCNC(=O)c1cncc(Br)n1] |
| A8 | 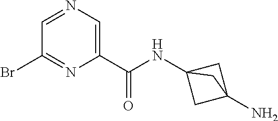 [SMILES: NC12CC(NC(=O)c3cncc(Br)n3)(C1)C2] |
| A9 | 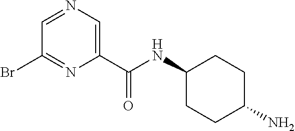 [SMILES: N[C@H]1CC[C@H](NC(=O)c2cncc(Br)n2)CC1] |
| A10 | 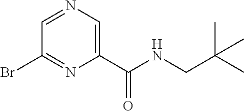 |

Method A: Intermediate B1: tert-Butyl 6-chloro-5-fluoro-3-methyl-2-(4,4,5,5-tetramethyl-1,3,2-dioxaborolan-2-yl)-1H-indole-1-carboxylate

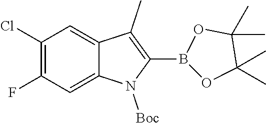 [SMILES: Cc1c(B2OC(C)(C)C(C)(C)O2)n(C(=O)OC(C)(C)C)c2cc(F)c(Cl)cc12]

The title compound was prepared in the following way:

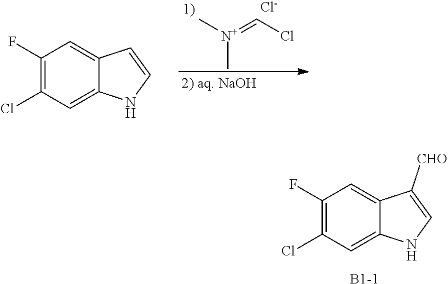

To a solution of 6-chloro-5-fluoro-1H-indole (5.05 g, 29.8 mmol) in CH₃CN (20 mL) was added a suspension of N-(chloromethylene)-N-methylmethanaminium chloride (5.72 g, 44.7 mmol) in CH₃CN (10 mL). The reaction mixture was stirred at rt for 20 min. TLC indicated full conversion. An aq. NaOH (4.76 g of NaOH in 60 mL of water) was added and the mixture was stirred at 100° C. for 20 min. After cooling under ice-bath, water was added to the reaction mixture. The resulting precipitate was collected by filtration, washed with water and CH₃CN, and dried. The filtrate was poured into a separate funnel with water and CH₂Cl₂. The resulting precipitate was collected by filtration, washed with water, and dried. The filtrate was extracted with CH₂Cl₂, washed with water and brine. The organic layer was dried over Na₂SO₄, filtered, and concentrated in vacuo. The obtained product from the filtration and the residue from aq. work-up was triturated with CH₃CN, collected by filtration, washed with CH₃CN and Et₂O, and dried to afford 6-chloro-5-fluoro-1H-indole-3-carbaldehyde (4.6 g, 78% yield). LCMS (ESI) m/z calcd for C₉H₅ClFNO 197.0, found 197.9 (M+H)⁺.

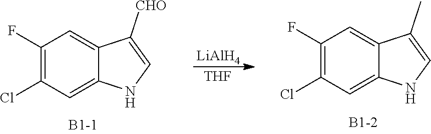

To a suspension of Compound B1-1 (4.6 g, 23.28 mmol) in THF (30 mL) under ice-water bath was added a solution of LiAlH₄ in THF (18.2 mL, 41.9 mmol, 2.3 M in THF). The reaction mixture was stirred at rt for 20 min and at 80° C. for 30 min. The reaction was quenched with Na₂SO₄·10H₂O under ice-water bath, then 2 M aq. NaOH and water were added. The resulting mixture was filtered through the celite pad, and the Al(OH)₃ product was washed with EtOAc. The combined filtrate was concentrated in vacuo. The residue was purified by flash chromatography over silica gel (EtOAc/heptane gradient 0 to 20%) to afford 6-chloro-5-fluoro-3-methyl-1H-indole (4.53 g, 23.28 mmol). ¹H NMR (500 MHz, DMSO-d₆) δ 10.94 (s, 1H), 7.48 (d, J=6.4 Hz, 1H), 7.45 (d, J=10.2 Hz, 1H), 7.23 (t, J=1.7 Hz, 1H), 2.22 (d, J=1.0 Hz, 3H). ¹⁹F NMR (470 MHZ, DMSO-d₆) δ −129.1.

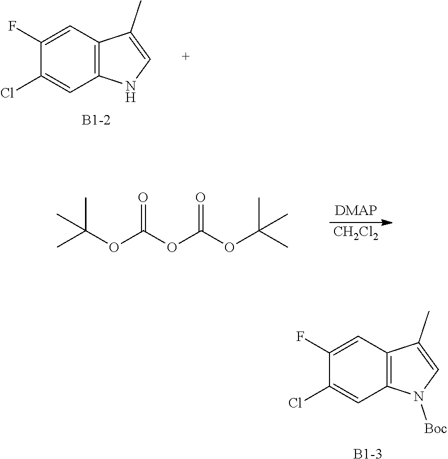

To a solution of Compound B1-2 (4.53 g, 24.67 mmol) in CH₂Cl₂ (10 mL) was added DMAP (0.117 g, 0.958 mmol) and a solution of BOC₂O (5.38 g, 24.67 mmol) in CH₂Cl₂ (5 mL). The reaction mixture was stirred at rt for 40 min. After the bulk of CH₂Cl₂ was concentrated, a solution of Boc₂O (2 g, 10.89 mmol) in CH₂Cl₂ (10 mL) was added. The reaction mixture was stirred at rt for 20 min. The reaction mixture was concentrated in vacuo. The obtained product was triturated with MeOH—CH₃CN, collected by filtration, washed with MeOH. The filtrate was concentrated in vacuo. The residue was purified by flash chromatography over silica gel (EtOAc/heptane gradient 0 to 10%) to afford the desired product, which was combined with the product obtained by trituration, and dried to give tert-butyl 6-chloro-5-fluoro-3-methyl-1H-indole-1-carboxylate (5.79 g, 74% yield). ¹H NMR (500 MHZ, DMSO-d₆) δ 8.12 (d, J=6.6 Hz, 1H), 7.65 (dd, J=9.5, 1.8 Hz, 1H), 7.57 (s, 1H), 2.22 (s, 3H), 1.62 (s, 9H). ¹⁹F NMR (470 MHZ, DMSO-d₆) δ −123.2.

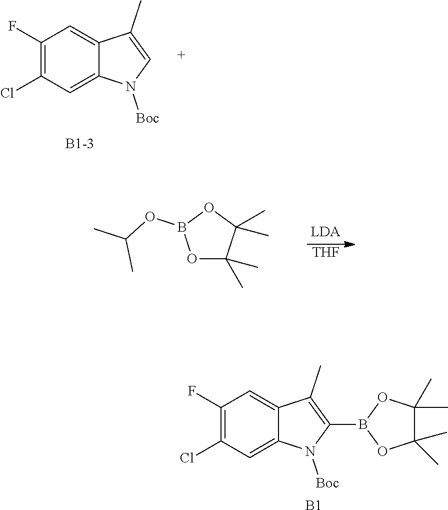

To a solution of Compound B1-3 (1.97 g, 6.94 mmol) in THF (20 mL) was added 2-isopropoxy-4,4,5,5-tetramethyl-1,3,2-dioxaborolane (2.13 mL, 10.41 mmol). After cooling to −78° C., LDA (6 mL, 12.00 mmol, 2.0 M in THF/heptane/ethylbenzene) was added to this reaction mixture via syringe. The reaction mixture was stirred at −78° C. to −22° C. for 50 min. The reaction was quenched with aq. KHSO₄, and extracted with EtOAc. The organic layer was washed with water and brine. The combined aq. layers were extracted with EtOAc. The organic extracts were combined, dried over Na₂SO₄, filtered, and concentrated in vacuo. The residue was purified by flash chromatography over silica gel (EtOAc/heptane gradient 0 to 10%) to afford the intermediate B1 (2.34 g, 82% yield). ¹H NMR (500 MHZ, DMSO-d₆) δ 7.92 (d, J=6.3 Hz, 1H), 7.64 (d, J=9.3 Hz, 1H), 2.22 (s, 3H), 1.65 (s, 9H), 1.34 (s, 12H). ¹⁹F NMR (470 MHZ, DMSO-d₆) δ −123.3.

### Method B: Intermediate B2: tert-Butyl 3-methyl-5-(pentafluoro-λ⁶-sulfaneyl)-2-(4,4,5,5-tetramethyl-1,3,2-dioxaborolan-2-yl)-1H-indole-1-carboxylate

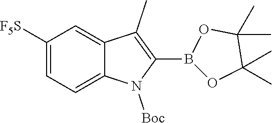

The title compound was prepared in the following way:

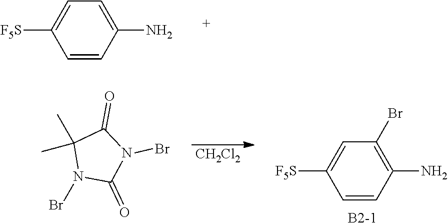

To a solution of 4-(pentafluoro-λ⁶-sulfaneyl) aniline (5.12 g, 23.36 mmol) in CH₂Cl₂ at 0° C. was added 1,3-dibromo-5,5-dimethylimidazolidine-2,4-dione (3.34 g, 11.68 mmol). The reaction mixture was stirred at 0° C. for 45 min, then at rt for 45 min. The reaction mixture was filtered through celite pad, and washed with CH₂Cl₂. The filtrate was concentrated in vacuo. The residue was purified by flash chromatography over silica gel (EtOAc/heptane gradient 0 to 50%) to afford 2-bromo-4-(pentafluoro-λ⁶-sulfaneyl) aniline (6.24 g, 90% yield). ¹H NMR (500 MHz, Chloroform-d) δ 7.81 (d, J=2.5 Hz, 1H), 7.49 (dd, J=8.9, 2.5 Hz, 1H), 6.72 (dt, J=8.9, 1.0 Hz, 1H), 4.45 (s, 2H).

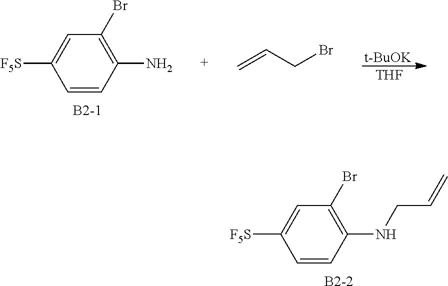

To a solution of Compound B2-1 (3.44 g, 11.54 mmol) in THF (40 mL) under ice-bath was added a solution of t-BuOK in THF (13.85 mL, 13.85 mmol, 1 M in THF) via syringe over 10 min. The reaction mixture was stirred at 0° C. for 15 min. To this mixture was added a solution of allyl bromide (1.2 mL, 13.85 mmol) in THF (11 mL) via syringe over 10 min. The resulting mixture was stirred at 0° C. for 15 min and at rt for 30 min. The reaction was quenched with water, and the bulk of THF was concentrated. The residue was poured into water, extracted with EtOAc. The organic layer was washed with water and brine. The combined aq. layers were extracted with EtOAc, and the organic layer was washed with brine. The combined organic extracts were dried over Na₂SO₄, filtered, and concentrated in vacuo. The residue was purified by flash chromatography over silica gel (EtOAc/heptane gradient 0 to 20%) to afford N-allyl-2-bromo-4-(pentafluoro-λ⁶-sulfaneyl) aniline (2.14 g, 55% yield) as a pale orange oil. ¹H NMR (500 MHZ, DMSO-d₆) δ 7.88 (d, J=2.6 Hz, 1H), 7.65 (dd, J=9.2, 2.7 Hz, 1H), 6.66 (d, J=9.2 Hz, 1H), 6.39 (t, J=6.0 Hz, 1H), 5.85 (ddt, J=17.2, 9.9, 4.7 Hz, 1H), 5.18-5.09 (m, 2H), 3.90 (ddd, J=6.5, 4.4, 2.1 Hz, 2H). LCMS (ESI) m/z calcd for C₉H₉⁸¹BrF₅NS 339.0, found 340.0 (M+H)⁺.

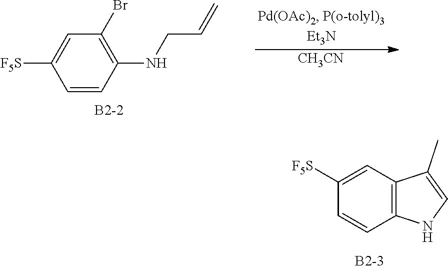

Compound B2-2 (4.485 g, 13.26 mmol), tri-o-tolylphosphine (161 mg, 0.531 mmol), and Pd(OAc)₂ (44.7 mg, 0.199 mmol) were placed in a 250 mL round-bottom flask. The flask was purged with N₂. To this mixture was added CH₃CN (40 mL) followed by Et₃N (2.8 mL, 19.9 mmol). The reaction mixture was purged with N₂ again and was stirred at 90° C. for 8 h under N₂ (balloon). The reaction mixture was filtered, washed with heptane/EtOAc, and the filtrate was concentrated in vacuo. The residue was purified by flash chromatography over silica gel (EtOAc/heptane gradient 0 to 20%) to afford 3-methyl-5-(pentafluoro-λ⁶-sulfaneyl)-1H-indole (2.44 g, 72% yield). ¹H NMR (500 MHZ, Chloroform-d) δ 8.10 (s, 1H), 8.00 (d, J=2.2 Hz, 1H), 7.59 (dd, J=8.9, 2.2 Hz, 1H), 7.34 (d, J=9.0 Hz, 1H), 7.11-7.07 (m, 1H), 2.36 (t, J=0.9 Hz, 3H). LCMS (ESI) m/z calcd for C₉H₈F₅NS 257.0, found 258.1 (M+H)⁺.

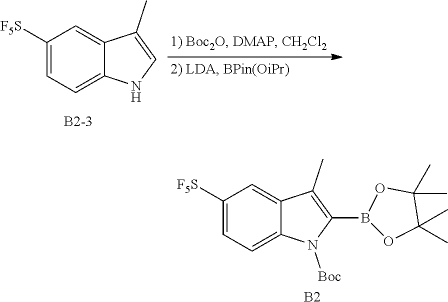

Intermediate B2 was prepared Compound B2-3 in a manner analogous to intermediate B1, tert-butyl 3-methyl-5-(pentafluoro-λ⁶-sulfaneyl)-2-(4,4,5,5-tetramethyl-1,3,2-dioxaborolan-2-yl)-1H-indole-1-carboxylate. ¹H NMR (500 MHZ, Chloroform-d) δ 7.93-7.82 (m, 2H), 7.66 (dd, J=8.9, 2.4 Hz, 1H), 2.32 (s, 3H), 1.68 (s, 9H), 1.43 (s, 12H).

### Method C: Intermediate B3: (1-(tert-Butoxycarbonyl)-6-chloro-7-fluoro-3-methyl-1H-indol-2-yl) boronic Acid

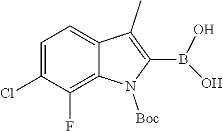 [SMILES: Cc1c(B(O)O)n(C(=O)OC(C)(C)C)c2c(F)c(Cl)ccc12]

The title compound was prepared in the following way:

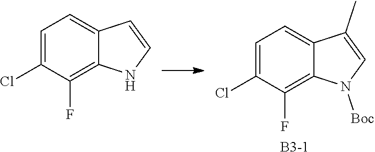

tert-Butyl 6-chloro-7-fluoro-3-methyl-1H-indole-1-carboxylate was prepared from 6-chloro-7-fluoro-1H-indole (CAS 259860-04-3) in a manner analogous to tert-butyl 6-chloro-5-fluoro-3-methyl-1H-indole-1-carboxylate. LCMS (ESI) m/z calcd for C₁₄H₁₅ClFNO₂ 283.1, found 228.0 (M+H-^{t}Bu)⁺.

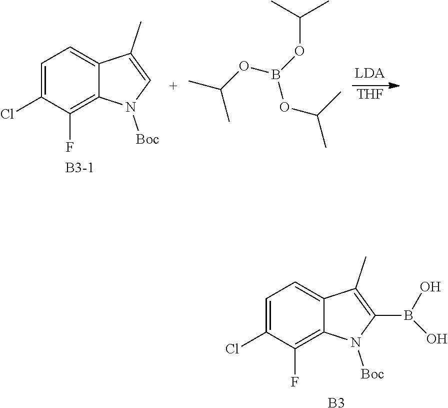

To a 500 mL flask containing Compound B3-1 (3.08 g, 10.86 mmol) was added THF (30 mL). The flask was put under an N₂ atmosphere, and triisopropyl borate (8 mL, 34.5 mmol) was added. The flask was then cooled to 0° C. in an ice-bath, and LDA (16 mL, 32.0 mmol, 2.0 M in THF/heptane/ethylbenzene) was added dropwise over 10 min. The reaction was then stirred at 0° C. for 1 h. An additional triisopropyl borate (1.3 mL, 5.60 mmol) and LDA (2.8 mL, 5.60 mmol) were added. After 5 min, the reaction was quenched reaction with 1 M aq. KHSO₄ (70 mL, pH tested to be ˜1), and the mixture was stirred at rt for 10 min. Two layers were separated, with copious amounts of precipitate seen in aq. layer. Extra water (˜50 mL) was added to redissolve all precipitated material and transferred to a separated funnel. The aq. layer was extracted with EtOAc (2×30 mL), and the organic extracts were dried over Na₂SO₄, filtered, and concentrated in vacuo to afford the intermediate B3 (3.48 g, 98% yield), which was directly used for the next reaction without further purification. LCMS (ESI) m/z calcd for C₁₄H₁₆BClFNO₄ 327.1, found 254.1 (M-^{t}BuO)⁺.

### Method D: Intermediate B4: 3,6-dimethyl-2-(4,4,5,5-tetramethyl-1,3,2-dioxaborolan-2-yl)-1H-indole

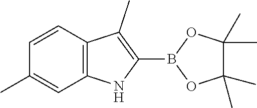

The title compound was prepared in the following way:

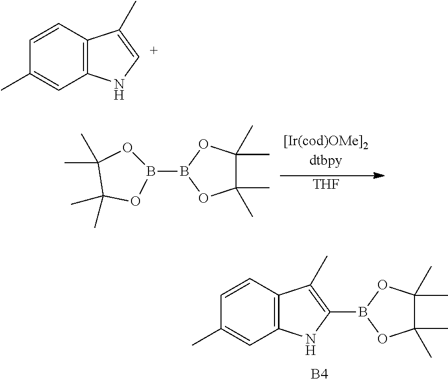

3,6-Dimethyl-1H-indole (102 mg, 0.702 mmol), B₂(Pin)₂ (95 mg, 0.374 mmol), [Ir(cod)OMe]₂ (2.3 mg, 0.0035 mmol), and dtbpy (1.9 mg, 0.0070 mmol) were placed in a 8 mL vial. The vial was purged by N₂, and THF (1.5 mL) was added. N₂ was bubbled into the reaction mixture. The reaction mixture was stirred at 90° C. for 3 h. The desired product MS peak was confirmed by LCMS. LCMS (ESI) m/z calcd for C₁₆H₂₂BNO₂ 271.2, found 272.2 (M+H)⁺. This reaction mixture (0.75 mL) was directly used for the next reaction.

Intermediates B5-B44 were prepared in a manner analogous to Intermediates B1-B4.

| Intermediate | Method | Structure |
|---|---|---|
| B5 | A | 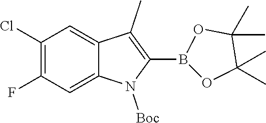 [SMILES: Cc1c(B2OC(C)(C)C(C)(C)O2)n(C(=O)OC(C)(C)C)c2cc(F)c(Cl)cc12] |
| B6 | A | 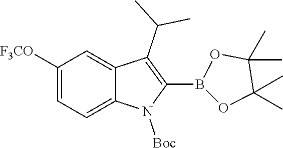 [SMILES: CC(C)c1c(B2OC(C)(C)C(C)(C)O2)n(C(=O)OC(C)(C)C)c2ccc(OC(F)(F)F)cc12] |
| B7 | B | 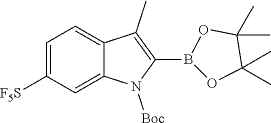 |
| B8 | C | 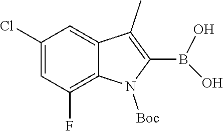 [SMILES: Cc1c(B(O)O)n(C(=O)OC(C)(C)C)c2c(F)cc(Cl)cc12] |
| B9 | C | 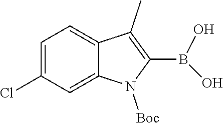 [SMILES: Cc1c(B(O)O)n(C(=O)OC(C)(C)C)c2cc(Cl)ccc12] |
| B10 | C | 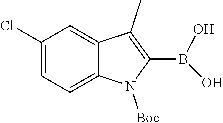 [SMILES: Cc1c(B(O)O)n(C(=O)OC(C)(C)C)c2ccc(Cl)cc12] |
| B11 | C | 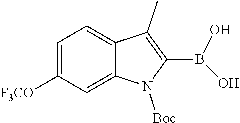 [SMILES: Cc1c(B(O)O)n(C(=O)OC(C)(C)C)c2cc(OC(F)(F)F)ccc12] |
| B12 | C | 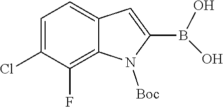 [SMILES: CC(C)(C)OC(=O)n1c(B(O)O)cc2ccc(Cl)c(F)c21] |
| B13 | C | 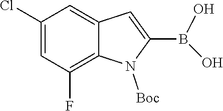 [SMILES: CC(C)(C)OC(=O)n1c(B(O)O)cc2cc(Cl)cc(F)c21] |
| B14 | C | 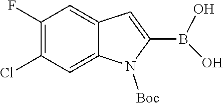 [SMILES: CC(C)(C)OC(=O)n1c(B(O)O)cc2cc(F)c(Cl)cc21] |
| B15 | C | 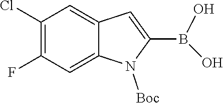 [SMILES: CC(C)(C)OC(=O)n1c(B(O)O)cc2cc(Cl)c(F)cc21] |
| B16 | C | 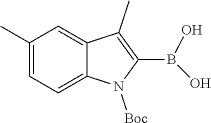 [SMILES: Cc1ccc2c(c1)c(C)c(B(O)O)n2C(=O)OC(C)(C)C] |
| B17 | C | 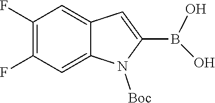 [SMILES: CC(C)(C)OC(=O)n1c(B(O)O)cc2cc(F)c(F)cc21] |
| B18 | C | 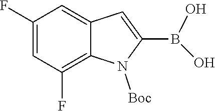 [SMILES: CC(C)(C)OC(=O)n1c(B(O)O)cc2cc(F)cc(F)c21] |
| B19 | C | 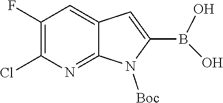 [SMILES: CC(C)(C)OC(=O)n1c(B(O)O)cc2cc(F)c(Cl)nc21] |
| B20 | C | 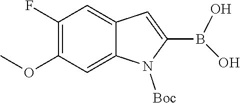 [SMILES: COc1cc2c(cc1F)cc(B(O)O)n2C(=O)OC(C)(C)C] |
| B21 | C | 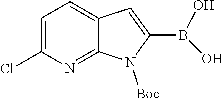 [SMILES: CC(C)(C)OC(=O)n1c(B(O)O)cc2ccc(Cl)nc21] |
| B22 | C | 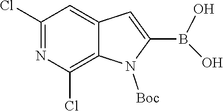 [SMILES: CC(C)(C)OC(=O)n1c(B(O)O)cc2cc(Cl)nc(Cl)c21] |
| B23 | C | 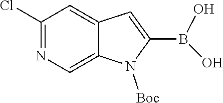 [SMILES: CC(C)(C)OC(=O)n1c(B(O)O)cc2cc(Cl)ncc21] |
| B24 | C | 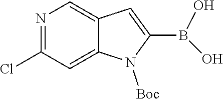 [SMILES: CC(C)(C)OC(=O)n1c(B(O)O)cc2cnc(Cl)cc21] |
| B25 | C | 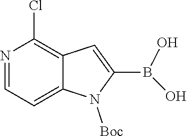 [SMILES: CC(C)(C)OC(=O)n1c(B(O)O)cc2c(Cl)nccc21] |
| B26 | C | 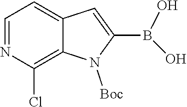 [SMILES: CC(C)(C)OC(=O)n1c(B(O)O)cc2ccnc(Cl)c21] |
| B27 | D | 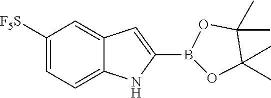 |
| B28 | D | 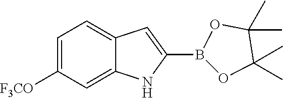 [SMILES: CC1(C)OB(c2cc3ccc(OC(F)(F)F)cc3[nH]2)OC1(C)C] |
| B29 | D | 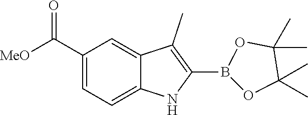 [SMILES: COC(=O)c1ccc2[nH]c(B3OC(C)(C)C(C)(C)O3)c(C)c2c1] |
| B30 | D | 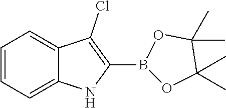 [SMILES: CC1(C)OB(c2[nH]c3ccccc3c2Cl)OC1(C)C] |
| B31 | D | 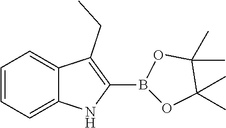 [SMILES: CCc1c(B2OC(C)(C)C(C)(C)O2)[nH]c2ccccc12] |
| B32 | D | 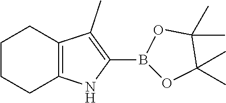 [SMILES: Cc1c(B2OC(C)(C)C(C)(C)O2)[nH]c2c1CCCC2] |
| B33 | D | 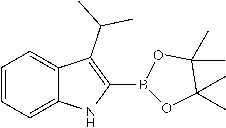 [SMILES: CC(C)c1c(B2OC(C)(C)C(C)(C)O2)[nH]c2ccccc12] |
| B34 | D | 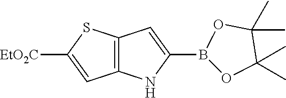 [SMILES: CCOC(=O)c1cc2[nH]c(B3OC(C)(C)C(C)(C)O3)cc2s1] |
| B35 | D | 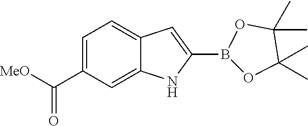 [SMILES: COC(=O)c1ccc2cc(B3OC(C)(C)C(C)(C)O3)[nH]c2c1] |
| B36 | D | 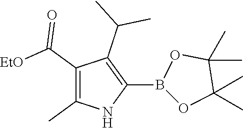 [SMILES: CCOC(=O)c1c(C)[nH]c(B2OC(C)(C)C(C)(C)O2)c1C(C)C] |
| B37 | D | 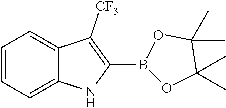 [SMILES: CC1(C)OB(c2[nH]c3ccccc3c2C(F)(F)F)OC1(C)C] |
| B38 | D | 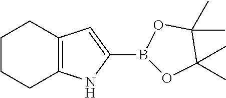 [SMILES: CC1(C)OB(c2cc3c([nH]2)CCCC3)OC1(C)C] |
| B39 | D | 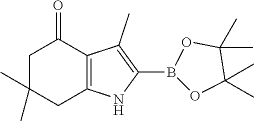 |
| B40 | D | 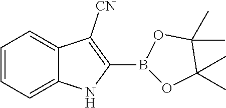 [SMILES: CC1(C)OB(c2[nH]c3ccccc3c2C#N)OC1(C)C] |
| B41 | D | 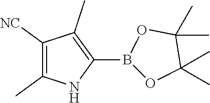 |
| B42 | D | 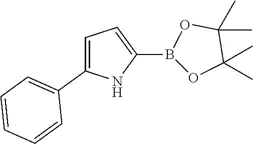 [SMILES: CC1(C)OB(c2ccc(-c3ccccc3)[nH]2)OC1(C)C] |
| B43 | D | 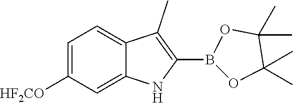 |
| B44 | D | 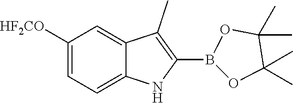 |

### SYNTHESIS OF THE EXAMPLES

### Example 1: N-(2-amino-2-methylpropyl)-6-(6-chloro-5-fluoro-3-methyl-1H-indol-2-yl)pyrazine-2-carboxamide

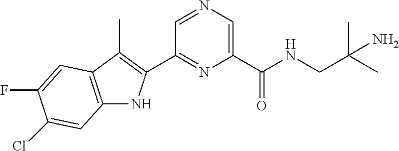 [SMILES: Cc1c(-c2cncc(C(=O)NCC(C)(C)N)n2)[nH]c2cc(Cl)c(F)cc12]

The title compound was prepared in the following way:

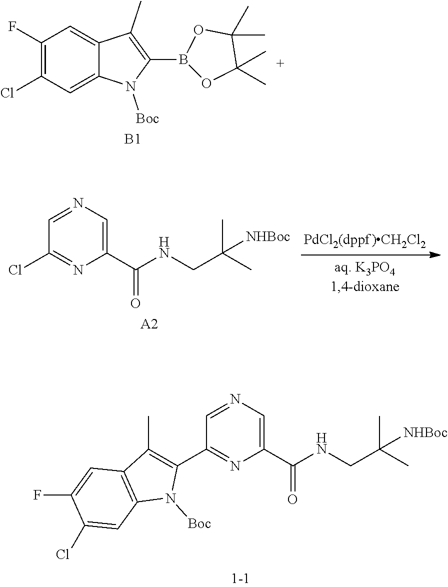

Compound B1 (3.34 g, 8.15 mmol), Compound A2 (2.1 g, 6.39 mmol), and PdCl₂(dppf)·CH₂Cl₂ (0.26 g, 0.32 mmol) were placed in a 100 mL round bottom flask. To this mixture was added 1,4-dioxane (10 mL) followed by aq. K₃PO₄ (9.6 mL, 19.16 mmol, 2 M). The flask was evacuated and back-filled with N₂, then the reaction mixture was stirred at 100° C. for 45 min. After dilution with EtOAc, the mixture was washed with water, aq. N-acetyl-cysteine (40 mL, 500 mg dissolved in 50 ml of water), and washed with sat. NaHCO₃. The organic layer was dried over Na₂SO₄, filtered, and concentrated in vacuo. The residue was purified by flash chromatography over silica gel (EtOAc/heptane gradient 0 to 50%) to afford the coupling product, which was further purified by trituration with MeOH—CH₃CN (sonication and stirred at rt overnight) to afford tert-butyl 2-(6-((2-((tert-butoxycarbonyl)amino)-2-methylpropyl)carbamoyl)pyrazin-2-yl)-6-chloro-5-fluoro-3-methyl-1H-indole-1-carboxylate (2.06 g, 56% yield). LCMS (ESI) m/z calcd for C₂₈H₃₅ClFN₅O₅ 575.2, found 576.3 (M+H)⁺.

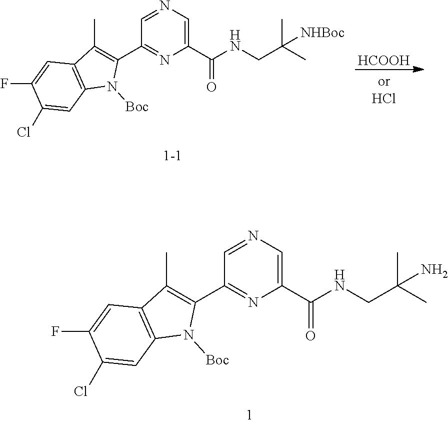 [SMILES: Cc1c(-c2cncc(C(=O)NCC(C)(C)NC(=O)OC(C)(C)C)n2)n(C(=O)OC(C)(C)C)c2cc(Cl)c(F)cc12 ; "HCOOH" ; Cc1c(-c2cncc(C(=O)NCC(C)(C)N)n2)n(C(=O)OC(C)(C)C)c2cc(Cl)c(F)cc12]

1) HCOOH procedure: A mixture of Compound 1-1 (1.93 g, 3.35 mmol) in HCOOH (10.1 mL, 268 mmol) was stirred at 70° C. for 7 h, then left at rt overnight. The same reaction was repeated with another two batches (167 mg, 0.29 mmol, and 1.618 g, 2.81 mmol). These reaction mixture was combined and concentrated in vacuo. The residue was basified with aq. NaOH. The resulting suspension was sonicated and stirred well to make complete free form. To this suspension was added MeOH and the resulting slurry was stirred at rt for 30 min (occasionally sonicated), then filtered and washed with water, and dried. The obtained product was suspended in MeOH—CH₃CN, and the resulting slurry was stirred at rt for 10 min (occasionally sonicated), then filtered, washed with MeOH—CH₃CN, and dried to afford the title compound (2.17 g, 89% yield) as a free base form. ¹H NMR (500 MHZ, DMSO-d₆) δ 11.97 (s, 1H), 9.30 (s, 1H), 9.07 (s, 1H), 8.91-8.80 (m, 1H), 7.74 (d, J=10.0 Hz, 1H), 7.64-7.58 (m, 1H), 3.31 (d, J=7.5 Hz, 2H), 2.65 (s, 3H), 1.07 (s, 6H). ¹⁹F NMR (470 MHz, DMSO-d₆) δ −127.4. LCMS (ESI) m/z calcd for C₁₈H₁₉ClFN₅O 375.1, found 376.2 (M+H)⁺.

2) HCl procedure: To a 100 mL round bottom flask containing Compound 1-1 (167 mg, 0.290 mmol) was added 4 M HCl in 1,4-dioxane (3 mL, 12.00 mmol) and the reaction was stirred at rt for 69 h. The resulting product was collected by filtration, washed with CH₃CN, and dried to afford to the title compound (105 mg, 86% yield) as a HCl salt form. ¹H NMR (500 MHZ, DMSO-d₆) δ 12.33 (s, 1H), 9.59 (t, J=6.6 Hz, 1H), 9.34 (s, 1H), 9.10 (s, 1H), 8.01 (s, 3H), 7.75 (d, J=10.1 Hz, 1H), 7.65 (d, J=6.3 Hz, 1H), 3.60 (d, J=6.6 Hz, 2H), 2.66 (s, 3H), 1.32 (s, 6H). ¹⁹F NMR (470 MHZ, DMSO-d₆) δ −127.5. LCMS (ESI) m/z calcd for C₁₈H₁₉ClFN₅₀ 375.1 found 376.0 [M+H]⁺.

### Example 2: N-(2-Amino-2-methylpropyl)-6-(5-(trifluoromethoxy)-1H-indol-2-yl)pyrazine-2-carboxamide

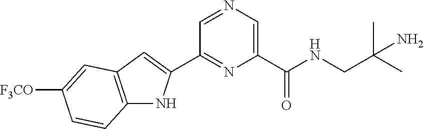 [SMILES: CC(C)(N)CNC(=O)c1cncc(-c2cc3cc(OC(F)(F)F)ccc3[nH]2)n1]

The title compound was prepared in the following way:

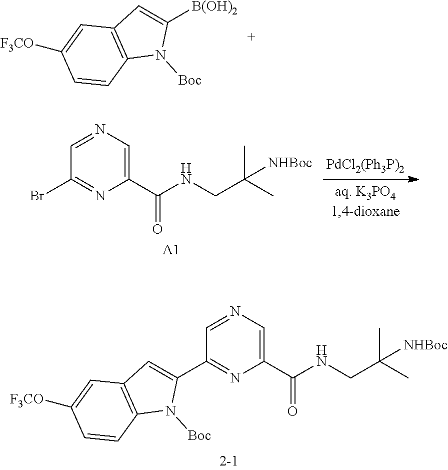

Compound A1 (190 mg, 0.509 mmol), (1-(tert-butoxycarbonyl)-5-(trifluoromethoxy)-1H-indol-2-yl) boronic acid (211 mg, 0.611 mmol, CAS 1034566-16-9), and PdCl₂(Ph₃P)₂ (18 mg, 0.025 mmol) were placed in a vial. The vial was purged with N₂, and capped. To this mixture were added 1,4-dioxane (0.3 mL) and aq. K₃PO₄ (0.76 mL, 1.527 mmol, 2 M). The reaction mixture was stirred at 100° C. for 30 min. The reaction mixture was directly loaded to silica gel and purified by flash chromatography over silica gel (EtOAc/heptane gradient 0 to 50%) to afford tert-butyl 2-(6-((2-((tert-butoxycarbonyl)amino)-2-methylpropyl)carbamoyl)pyrazin-2-yl)-5-(trifluoromethoxy)-1H-indole-1-carboxylate (204 mg, 68% yield). LCMS (ESI) m/z calcd for C₂₈H₃₄F₃N₅O₆ 593.3, found 594.3 (M+H)⁺.

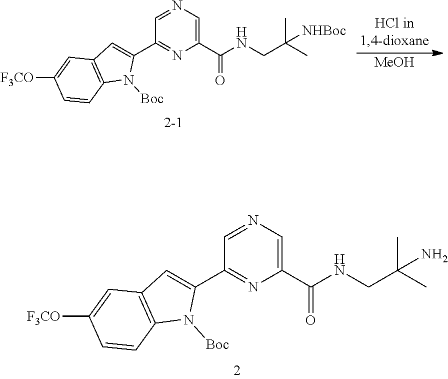 [SMILES: "MeOH" ; CC(C)(CNC(=O)c1cncc(-c2cc3cc(OC(F)(F)F)ccc3n2C(=O)OC(C)(C)C)n1)NC(=O)OC(C)(C)C ; CC(C)(N)CNC(=O)c1cncc(-c2cc3cc(OC(F)(F)F)ccc3n2C(=O)OC(C)(C)C)n1]

To a solution of Compound 2-1 (204 mmol) in MeOH (2 mL) was added HCl in 1,4-dioxane (5 mL, 20 mmol, 4 M). The reaction mixture was stirred at rt for 39 h. The reaction mixture was concentrated in vacuo. The residue was triturated with MeOH—CH₃CN, collected by filtration, washed with CH₃CN, and dried to afford the title compound (144 mg, 97% yield). ¹H NMR (500 MHz, DMSO-d₆) δ12.96 (s, 1H), 9.90 (t, J=6.6 Hz, 1H), 9.53 (d, J=0.5 Hz, 1H), 9.06 (d, J=0.5 Hz, 1H), 8.24 (s, 3H), 7.67-7.62 (m, 2H), 7.54 (dd, J=2.2, 0.8 Hz, 1H), 7.20 (ddd, J=8.9, 2.4, 1.0 Hz, 1H), 3.62 (d, J=6.6 Hz, 2H), 1.33 (s, 6H). ¹⁹F NMR (470 MHZ, DMSO-d₆) δ −56.9. LCMS (ESI) m/z calcd for C₁₈H₁₈F₃N₅O₂ 393.1 found 394.1 [M+H]⁺.

### Example 3: N-(2-amino-2-methylpropyl)-6-(3-methyl-5-(pentafluoro-λ⁶-sulfaneyl)-1H-indol-2-yl)pyrazine-2-carboxamide

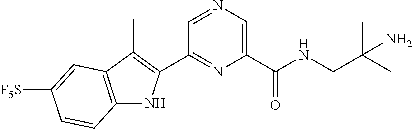

The title compound was prepared in the following way:

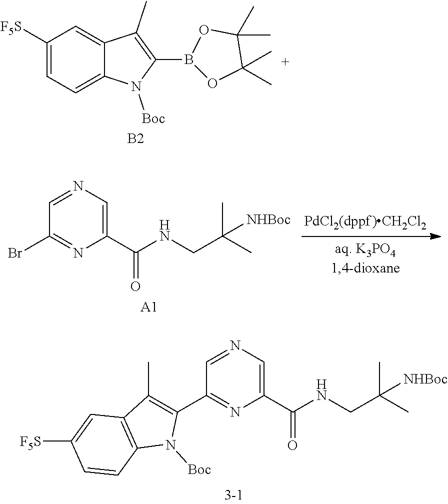

Compound B2 (470 mg, 0.972 mmol), Compound A1 (300 g, 0.804 mmol), and PdCl₂(dppf)·CH₂Cl₂ (0.033 g, 0.040 mmol) were placed in a 100 mL round bottom flask. To this mixture was added 1,4-dioxane (2 mL) followed by aq. K₃PO₄ (1.2 mL, 2.4 mmol, 2 M). The flask was evacuated and back-filled with N₂, then the reaction mixture was stirred at 100° C. for 30 min. The reaction mixture was diluted with EtOAc, washed with water and brine. The aq. layer was extracted with EtOAc. The combined organic extracts were dried over Na₂SO₄, filtered, and concentrated in vacuo. The residue was purified by flash chromatography over silica gel (EtOAc/heptane gradient 0 to 50%) to afford tert-butyl 2-(6-((2-((tert-butoxycarbonyl)amino)-2-methylpropyl)carbamoyl)pyrazin-2-yl)-3-methyl-5-(pentafluoro-λ⁶-sulfaneyl)-1H-indole-1-carboxylate (448 mg, 84% yield). LCMS (ESI) m/z calcd for C₂₈H₃₆F₅N₅O₅S 649.2, found 650.3 (M+H)⁺.

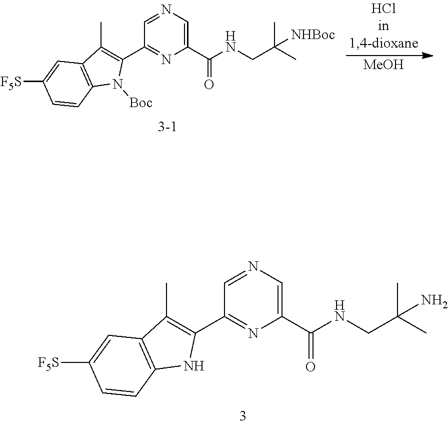

To a suspension of tert-butyl 2-(6-((2-((tert-butoxycarbonyl)amino)-2-methylpropyl)carbamoyl)pyrazin-2-yl)-3-methyl-5-(pentafluoro-λ⁶-sulfaneyl)-1H-indole-1-carboxylate (155 mg, 0.239 mmol) in MeOH (5 mL) was added HCl in 1,4-dioxane (6 mL, 24 mmol, 4 M). The reaction mixture was stirred at rt for 62 h, and concentrated in vacuo. The residue was triturated with MeOH—CH₃CN, collected by filtration, washed with MeOH—CH₃CN, and dried to afford the title compound (89 mg, 76% yield) as a HCl salt form. ¹H NMR (500 MHZ, DMSO-d₆) δ 12.37 (s, 1H), 9.46 (d, J=6.9 Hz, 1H), 9.40 (s, 1H), 9.14 (s, 1H), 8.31 (d, J=2.1 Hz, 1H), 7.92 (s, 3H), 7.73 (dd, J=9.0, 2.2 Hz, 1H), 7.67 (d, J=8.9 Hz, 1H), 3.60 (d, J=6.7 Hz, 2H), 2.76 (s, 3H), 1.33 (s, 6H). ¹⁹F NMR (470 MHZ, DMSO-d₆) δ 66.9. LCMS (ESI) m/z calcd for C₁₈H₂₀F₅N₅OS 449.1 found 450.3 [M+H]⁺.

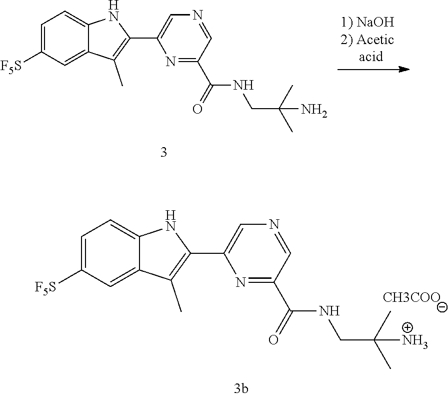

Step 1: A round bottom flask containing crude HCl salt (from a 50 g, 77 mmol deprotection of 3-1) was charged with water (1.25 L) and the resulting solution was brought to a pH between 9-14 by the addition of a 10% NaOH solution (150 mL). The mixture was stirred for 5 hours and the resulting slurry was filtered, washed with water and dried to afford N-(2-amino-2-methylpropyl)-6-(3-methyl-5-(pentafluoro-λ⁶-sulfaneyl)-1H-indol-2-yl)pyrazine-2-carboxamide as a free base (3a) (30.5 g, 88% yield).

Step 2: A round bottom flask was charged with N-(2-amino-2-methylpropyl)-6-(3-methyl-5-(pentafluoro-λ⁶-sulfaneyl)-1H-indol-2-yl)pyrazine-2-carboxamide (3a) (50 g, 111 mmol) and taken up in EtOH (150 mL). The mixture was warmed to 50° C. and treated with siliabond thiol resin (5 g) and stirred overnight. The mixture was cooled to room temperature, treated with Jacobi carbon (5 g) and warmed to 50° C. for 4 hours. The mixture was and filtered at 50° C. through celite, washed with additional warm EtOH and the filtrate transferred to a round bottom flask. The filtrate was diluted with water (50 mL), the mixture warmed to 50° C. and treated with 5 mL of a 10% solution of acetic acid in ethanol/water (96:4). Stirred for 2 hours at 50° C. and slowly cooled to 10° C. The resulting slurry was collected by filtration, washed with wet ethanol (96:4) and dried to give N-(2-amino-2-methylpropyl)-6-(3-methyl-5-(pentafluoro-λ⁶-sulfaneyl)-1H-indol-2-yl)pyrazine-2-carboxamide acetate (3b) (48 g, 85% yield). ¹H NMR (500 MHZ, DMSO-d₆) δ 12.83 (s, 1H), 9.60 (m, 1H), 9.36 (s, 1H), 9.09 (s, 1H), 8.27 (d, J=2.2 Hz, 1H), 7.70 (dd, J=9.0, 2.2 Hz, 1H), 7.63 (d, J=9.0 Hz, 1H), 3.49-3.33 (m, 2H), 2.74 (s, 3H), 1.91 (s, 3H), 1.16 (s, 6H). ¹⁹F NMR (470 MHZ, DMSO-d₆) δ 67.21, 90.85. LCMS (ESI) m/z calcd for C₁₈H₂₀F₅N₅OS 449.1 found 450.14 [M+H]⁺.

Examples 4-70 were prepared from the appropriate boronic acid or boronate ester and halo-pyrazine amide in a manner analogous to Examples 1-3.

| Example | Structure | NMR | LC-MS |
|---|---|---|---|
| 4 | 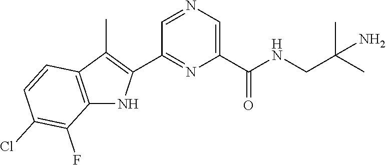 [SMILES: Cc1c(-c2cncc(C(=O)NCC(C)(C)N)n2)[nH]c2c(F)c(Cl)ccc12] | ¹H NMR (500 MHz, DMSO- d₆) δ 12.23 (s, 1H), 9.58 (t, J = 6.7 Hz, 1H), 9.37 (s, 1H), 9.12 (s, 1H), 8.05 (s, 3H), 7.58 (d, J = 8.6 Hz, 1H), 7.19 (dd, J = 8.6, 6.4 Hz, 1H), 3.61 (d, J = 6.6 Hz, 2H), 2.69 (s, 3H), 1.33 (s, 6H). ¹⁹F NMR (470 MHz, DMSO- | MS m/z calcd for C₁₈H₁₉ClFN₅O 375.1 found 376.2 [M + H]⁺ |
| | N-(2-amino-2-methylpropyl)- | d₆) δ -34.2 | |
| | 6-(6-chloro-7-fluoro-3-methyl- | | |
| | 1H-indol-2-yl)pyrazine-2- | | |
| | carboxamide | | |
| 5 | 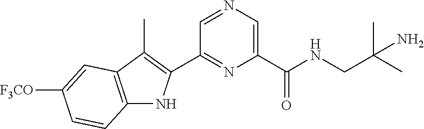 [SMILES: Cc1c(-c2cncc(C(=O)NCC(C)(C)N)n2)[nH]c2ccc(OC(F)(F)F)cc12] | ¹H NMR (500 MHz, DMSO- d₆) δ 12.38 (s, 1H), 9.66 (t, J = 6.6 Hz, 1H), 9.36 (s, 1H), 9.10 (s, 1H), 8.07 (s, 3H), 7.70 (d, J = 2.3 Hz, 1H), 7.62 (d, J = 8.8 Hz, 1H), 7.24-7.19 (m, 1H), 3.61 (d, J = 6.6 Hz, 2H), | MS m/z calcd for C₁₉H₂₀F₃N₅O₂ 407.2 found 408.1 [M + H]⁺ |
| | N-(2-amino-2-methylpropyl)- | 2.69 (s, 3H), 1.33 (s, 6H). | |
| | 6-(3-methyl-5- | ¹⁹F NMR (470 MHz, DMSO- | |
| | (trifluoromethoxy)-1H-indol-2- | d₆) δ -55.8 | |
| | yl)pyrazine-2-carboxamide | | |
| 6 | 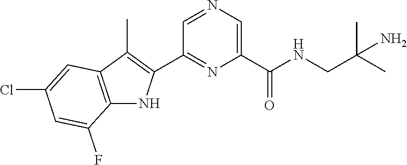 [SMILES: Cc1c(-c2cncc(C(=O)NCC(C)(C)N)n2)[nH]c2c(F)cc(Cl)cc12] | ¹H NMR (400 MHz, DMSO- d₆) δ 12.47 (s, 1H), 9.75 (s, 1H), 9.37 (s, 1H), 9.12 (s, 1H), 8.22 (s, 2H), 7.67 (d, J = 1.7 Hz, 1H), 7.23 (dd, J 10.9, 1.7 Hz, 1H), 3.59 (d, J = 6.6 Hz, 2H), 2.67 (s, 3H), 1.32 (s, 6H). ¹⁹F NMR (376 MHz, DMSO- d₆) δ -128.8 | MS m/z calcd for C₁₈H₁₉ClFN₅O 375.1 found 376.1 [M + H]⁺ |
| | N-(2-amino-2-methylpropyl)- | | |
| | 6-(5-chloro-7-fluoro-3-methyl- | | |
| | 1H-indol-2-yl)pyrazine-2- | | |
| | carboxamide | | |
| 7 | 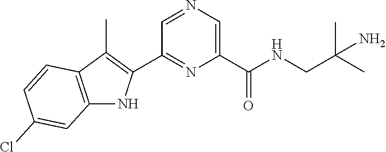 [SMILES: Cc1c(-c2cncc(C(=O)NCC(C)(C)N)n2)[nH]c2cc(Cl)ccc12] | ¹H NMR (500 MHz, DMSO- d₆) δ 12.55 (s, 1H), 9.82 (t, J = 6.6 Hz, 1H), 9.33 (s, 1H), 9.07 (s, 1H), 8.27- 8.12 (m, 3H), 7.71 (d, J = 8.5 Hz, 1H), 7.58 (d, J = 1.9 Hz, 1H), 7.08 (dd, J = 8.5, 1.9 Hz, 1H), 3.62 (d, J = 6.6 Hz, 2H), 2.68 (s, 3H), 1.33 (s, 6H). | MS m/z calcd for C₁₈H₂₀ClN₅O 357.1 found 358.3 [M + H]⁺ |
| | N-(2-amino-2-methylpropyl)- | | |
| | 6-(6-chloro-3-methyl-1H- | | |
| | indol-2-yl)pyrazine-2- | | |
| | carboxamide | | |
| 8 | 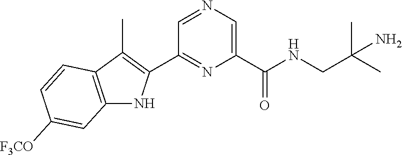 [SMILES: Cc1c(-c2cncc(C(=O)NCC(C)(C)N)n2)[nH]c2cc(OC(F)(F)F)ccc12] | ¹H NMR (400 MHz, DMSO- d₆) δ 11.95 (s, 1H), 9.38 (m, 1H), 9.35 (s, 1H), 9.10 (s, 1H), 7.91 (s, 3H), 7.82 (d, J = 8.7 Hz, 1H), 7.44 (s, 1H), 7.08 (dd, J = 8.7, 2.1 Hz, 1H), 3.59 (d, J = 6.7 Hz, 2H), 2.71 (s, 3H), 1.33 (s, 6H). | MS m/z calcd for C₁₉H₂₀F₃N₅O₂ 407.2 found 408.3 [M + H]⁺ |
| | N-(2-amino-2-methylpropyl)- | ¹⁹F NMR (376 MHz, DMSO- | |
| | 6-(3-methyl-6- | d₆) δ -56.8, -73.7 (TFA). | |
| | (trifluoromethoxy)-1H-indol-2- | | |
| | yl)pyrazine-2-carboxamide | | |
| 9 | 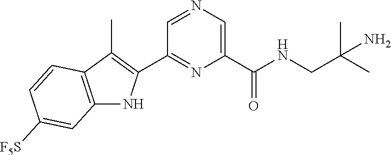 | ¹H NMR (400 MHz, DMSO- d₆) δ 12.77 (s, 1H), 9.66 (t, J = 6.7 Hz, 1H), 9.40 (s, 1H), 9.14 (s, 1H), 8.08- 7.97 (m, 4H), 7.89 (d, J = 8.9 Hz, 1H), 7.55 (dd, J = 8.9, 2.1 Hz, 1H), 3.60 (d, J = 6.6 Hz, 2H), 2.72 (s, 3H), 1.32 (s, 6H). | MS m/z calcd for C₁₈H₂₀F₅N₅OS 449.1 found 450.1 [M + H]⁺ |
| | N-(2-amino-2-methylpropyl)- | | |
| | 6-(3-methyl-6-(pentafluoro- | | |
| | □⁶-sulfaneyl)-1H-indol-2- | | |
| | yl)pyrazine-2-carboxamide | | |
| 10 | 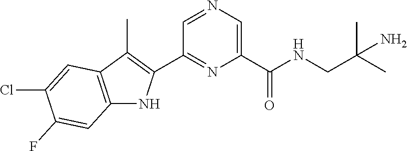 [SMILES: Cc1c(-c2cncc(C(=O)NCC(C)(C)N)n2)[nH]c2cc(F)c(Cl)cc12] | ¹H NMR (500 MHz, DMSO- d₆) δ 12.41 (s, 1H), 9.62 (t, J = 6.6 Hz, 1H), 9.33 (s,1H), 9.08 (s, 1H), 8.05 (s, 3H), 7.94 (d, J = 7.3 Hz, 1H), 7.47 (d, J = 9.8 Hz, 1H), 3.60 (d, J = 6.6 Hz, 2H), 2.67 (s, 3H), 1.32 (s, 6H). ¹⁹F NMR (470 MHz, DMSO- | MS m/z calcd for C₁₈H₁₉ClFN₅O 375.1 found 376.2 [M + H]⁺ |
| | N-(2-amino-2-methylpropyl)- | d₆) δ -121.1 | |
| | 6-(5-chloro-6-fluoro-3-methyl- | | |
| | 1H-indol-2-yl)pyrazine-2- | | |
| | carboxamide | | |
| 11 | 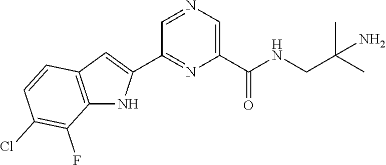 [SMILES: CC(C)(N)CNC(=O)c1cncc(-c2cc3ccc(Cl)c(F)c3[nH]2)n1] | ¹H NMR (400 MHz, DMSO- d₆) δ 12.54 (s, 1H), 9.76 (t, J = 6.6 Hz, 1H), 9.57 (s, 1H), 9.09 (s, 1H), 8.07 (s, 3H), 7.67 (t, J = 2.9 Hz, 1H), 7.52 (d, J = 8.3 Hz, 1H), 7.25-7.13 (m, 1H), 3.59 (d, J = 6.7 Hz, 2H), 1.32 (s, | MS m/z calcd for C₁₇H₁₇ClFN₅O 361.1 found 362.1 [M + H]⁺ |
| | N-(2-amino-2-methylpropyl)- | 6H). | |
| | 6-(6-chloro-7-fluoro-1H-indol- | ¹⁹F NMR (376 MHz, DMSO- | |
| | 2-yl)pyrazine-2-carboxamide | d₆) δ -133.5 | |
| 12 | 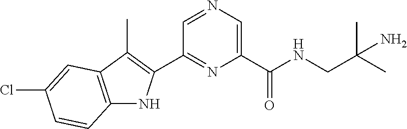 [SMILES: Cc1c(-c2cncc(C(=O)NCC(C)(C)N)n2)[nH]c2ccc(Cl)cc12] | ¹H NMR (500 MHz, DMSO- d₆) δ 12.52 (s, 1H), 9.81 (t, J = 6.6 Hz, 1H), 9.34 (s, 1H), 9.08 (s, 1H), 8.21 (s, 3H), 7.75 (d, J = 2.1 Hz, 1H), 7.57 (d, J = 8.6 Hz, 1H), 7.22 (dd, J = 8.6, 2.1 Hz, 1H), 3.62 (d, J = 6.6 Hz, 2H), 2.67 (s, 3H), 1.33 (s, | MS m/z calcd for C₁₈H₂₀ClN₅O 357.1 found 358.2 [M + H]⁺ |
| | N-(2-amino-2-methylpropyl)- | 6H). | |
| | 6-(5-chloro-3-methyl-1H- | | |
| | indol-2-yl)pyrazine-2- | | |
| | carboxamide | | |
| 13 | 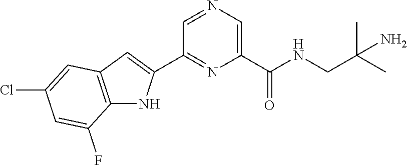 [SMILES: CC(C)(N)CNC(=O)c1cncc(-c2cc3cc(Cl)cc(F)c3[nH]2)n1] | ¹H NMR (400 MHz, DMSO- d₆) δ 12.59 (s, 1H), 9.80 (t, J = 6.7 Hz, 1H), 9.57 (s, 1H), 9.10 (s, 1H), 8.13 (s, 3H), 7.65-7.58 (m, 2H), 7.25 (dd, J = 10.9, 1.7 Hz, 1H), 3.60 (d, J = 6.7 Hz, 2H), 1.32 (s, 6H). | MS m/z calcd for C₁₇H₁₇ClFN₅O 361.1 found 362.2 [M + H]⁺ |
| | N-(2-amino-2-methylpropyl)- | ¹⁹F NMR (376 MHz, DMSO- | |
| | 6-(5-chloro-7-fluoro-1H-indol- | d₆) δ -127.7 | |
| | 2-yl)pyrazine-2-carboxamide | | |
| 14 | 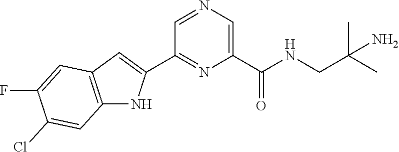 [SMILES: CC(C)(N)CNC(=O)c1cncc(-c2cc3cc(F)c(Cl)cc3[nH]2)n1] | ¹H NMR (400 MHz, DMSO- d₆) δ 12.86 (s, 1H), 9.79 (t, J = 6.6 Hz, 1H), 9.54 (s, 1H), 9.07 (s, 1H), 8.10 (s, 3H), 7.69 (d, J = 6.3 Hz, 1H), 7.67 (d, J = 3.1 Hz, 1H), 7.51 (d, J = 1.9 Hz, 1H), 3.60 (d, J = 6.7 Hz, 3H), 1.32 (s, 6H). | MS m/z calcd for C₁₇H₁₇ClFN₅O 361.1 found 362.1 [M + H]⁺ |
| | N-(2-amino-2-methylpropyl)- | ¹⁹F NMR (376 MHz, DMSO- | |
| | 6-(6-chloro-5-fluoro-1H-indol- | d₆) δ -127.0 | |
| | 2-yl)pyrazine-2-carboxamide | | |
| 15 | 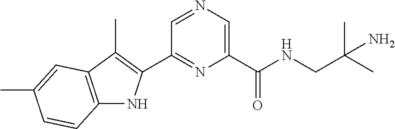 [SMILES: Cc1ccc2[nH]c(-c3cncc(C(=O)NCC(C)(C)N)n3)c(C)c2c1] | ¹H NMR (400 MHz, DMSO- d₆) δ 11.90 (s, 1H), 9.63 (t, J = 6.6 Hz, 1H), 9.30 (s, 1H), 9.02 (s, 1H), 8.08 (s, 3H), 7.45 (s, 1H), 7.41 (d, J = 8.3 Hz, 1H), 7.06 (d, J = 8.6 Hz, 1H), 3.59 (d, J = 6.6 Hz, 2H), 2.65 (s, 3H), 2.41 | MS m/z calcd for C₁₉H₂₃N₅O 337.2 found 338.1 [M + H]⁺ |
| | N-(2-amino-2-methylpropyl)- | (s, 3H), 1.32 (s, 6H). | |
| | 6-(3,5-dimethyl-1H-indol-2- | | |
| | yl)pyrazine-2-carboxamide | | |
| 16 | 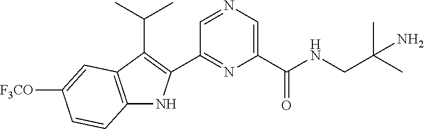 [SMILES: CC(C)c1c(-c2cncc(C(=O)NCC(C)(C)N)n2)[nH]c2ccc(OC(F)(F)F)cc12] | ¹H NMR (500 MHz, DMSO- d₆) δ 12.43 (s, 1H), 9.58 (t, J = 6.6 Hz, 1H), 9.23 (s, 1H), 9.13 (s, 1H), 8.21 (s, 3H), 7.80-7.75 (m, 1H), 7.66 (d, J = 8.9 Hz, 1H), 7.21 (ddt, J = 8.8, 2.1, 1.0 Hz, 1H), 3.77 (m, 1H), 3.62 | MS m/z calcd for C₂₁H₂₄F₃N₅O₂ 435.2 found 436.3 [M + H]⁺ |
| | N-(2-amino-2-methylpropyl)- | (d, J = 6.6 Hz, 2H), 1.52 (d, | |
| | 6-(3-isopropyl-5- | J = 7.0 Hz, 6H), 1.33 (s, | |
| | (trifluoromethoxy)-1H-indol-2- | 6H). | |
| | yl)pyrazine-2-carboxamide | ¹⁹F NMR (470 MHz, DMSO- | |
| | | d₆) δ -56.9 | |
| 17 | 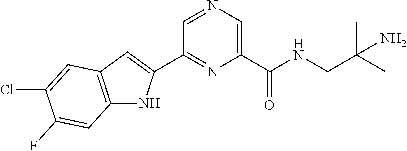 [SMILES: CC(C)(N)CNC(=O)c1cncc(-c2cc3cc(Cl)c(F)cc3[nH]2)n1] | ¹H NMR (400 MHz, DMSO- d₆) δ 12.54 (s, 2H), 9.59 (t, J = 6.8 Hz, 1H), 9.53 (s, 1H), 9.06 (s, 1H), 7.96 (s, 3H), 7.91 (d, J = 7.4 Hz, 1H), 7.53 (s, 1H), 7.52 (d, J = 9.9 Hz, 1H), 3.58 (d, J = 6.6 Hz, 3H), 1.32 (s, 6H). ¹⁹F NMR (376 MHz, DMSO- | MS m/z calcd for C₁₇H₁₇ClFN₅O 361.1 found 362.2 [M + H]⁺ |
| | N-(2-amino-2-methylpropyl)- | d₆) δ -120.9 | |
| | 6-(5-chloro-6-fluoro-1H-indol- | | |
| | 2-yl)pyrazine-2-carboxamide | | |
| 18 | 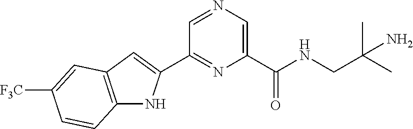 [SMILES: CC(C)(N)CNC(=O)c1cncc(-c2cc3cc(C(F)(F)F)ccc3[nH]2)n1] | ¹H NMR (500 MHz, DMSO- d₆) δ 12.88 (s, 1H), 9.77 (t, J = 6.6 Hz, 1H), 9.58 (s, 1H), 9.10 (s, 1H), 8.09 (s, 1H), 8.07 (s, 3H), 7.76 (d, J = 8.6 Hz, 1H), 7.68 (d, J = 3.0 Hz, 1H), 7.53 (dd, J = 8.6, 1.8 Hz, 1H), 3.62 (d, | MS m/z calcd for C₁₈H₁₈F₃N₅O 377.2 found 378.1 [M + H]⁺ |
| | N-(2-amino-2-methylpropyl)- | J = 6.6 Hz, 2H), 1.34 (s, 6H). | |
| | 6-(5-(trifluoromethyl)-1H- | ¹⁹F NMR (470 MHz, DMSO- | |
| | indol-2-yl)pyrazine-2- | d₆) δ -58.8 | |
| | carboxamide | | |
| 19 | 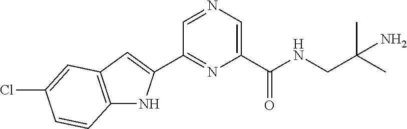 [SMILES: CC(C)(N)CNC(=O)c1cncc(-c2cc3cc(Cl)ccc3[nH]2)n1] | ¹H NMR (500 MHz, DMSO- d₆) δ 12.65 (s, 1H), 9.76 (t, J = 6.7 Hz, 1H), 9.53 (s, 1H), 9.07 (s, 1H), 8.09 (s, 3H), 7.73 (s, 1H), 7.58 (d, J = 8.7 Hz, 1H), 7.49 (s, 1H), 7.27-7.21 (m, 1H), 3.61 (d, J = 6.6 Hz, 2H), 1.33 (s, | MS m/z calcd for C₁₇H₁₈ClN₅O 343.1 found 344.0[M + H]⁺ |
| | N-(2-amino-2-methylpropyl)- | 6H). | |
| | 6-(5-chloro-1H-indol-2- | | |
| | yl)pyrazine-2-carboxamide | | |
| 20 | 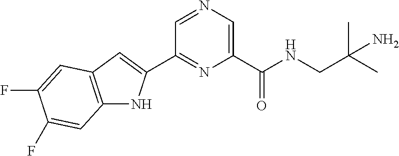 [SMILES: CC(C)(N)CNC(=O)c1cncc(-c2cc3cc(F)c(F)cc3[nH]2)n1] | ¹H NMR (400 MHz, DMSO- d₆) δ 12.66 (s, 1H), 9.71 (t, J = 6.7 Hz, 1H), 9.52 (s, 1H), 9.06 (s, 1H), 8.06 (s, 3H), 7.71 (dd, J = 11.1, 8.0 Hz, 1H), 7.53 (s, 1H), 7.52- 7.48 (m, 1H), 3.60 (d, J = 6.7 Hz, 2H), 1.33 (s, 6H). ¹⁹F NMR (376 MHz, DMSO- | MS m/z calcd for C₁₇H₁₇F₂N₅O 345.1 found 346.2 [M + H]⁺ |
| | N-(2-amino-2-methylpropyl)- | d₆) δ 141.6, -147.2 | |
| | 6-(5,6-difluoro-1H-indol-2- | | |
| | yl)pyrazine-2-carboxamide | | |
| 21 | 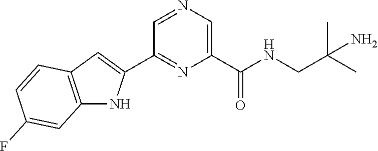 [SMILES: CC(C)(N)CNC(=O)c1cncc(-c2cc3ccc(F)cc3[nH]2)n1] | ¹H NMR (400 MHz, DMSO- d₆) δ 12.65 (s, 1H), 9.78 (t, J = 6.6 Hz, 1H), 9.49 (s, 1H), 9.02 (s, 1H), 8.14 (s, 3H), 7.67 (dd, J = 8.8, 5.5 Hz, 1H), 7.52 (s, 1H), 7.28 (dd, J = 10.0, 2.3 Hz, 1H), 6.95 (td, J = 9.3, 2.4 Hz, 1H), 3.60 (d, J = 6.6 Hz, | MS m/z calcd for C₁₇H₁₈FN₅O 327.2 found 328.2 [M + H]⁺ |
| | N-(2-amino-2-methylpropyl)- | 2H), 1.32 (s, 6H). | |
| | 6-(6-fluoro-1H-indol-2- | ¹⁹F NMR (376 MHz, DMSO- | |
| | yl)pyrazine-2-carboxamide | d₆) δ -118.4 | |
| 22 | 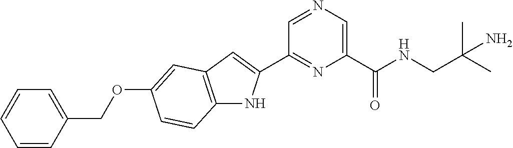 [SMILES: CC(C)(N)CNC(=O)c1cncc(-c2cc3cc(OCc4ccccc4)ccc3[nH]2)n1] | ¹H NMR (500 MHz, DMSO- d₆) δ 12.18 (s, 1H), 9.67 (t, J = 6.5 Hz, 1H), 9.47 (s, 1H), 9.01 (d, J = 1.7 Hz, 1H), 8.06 (s, 3H), 7.50 (d, J = 7.6 Hz, 2H), 7.47 (d, J = 8.9 Hz, 1H), 7.44-7.39 (m, 3H), 7.34 (t, J = 7.3 Hz, 1H), 7.22 (s, 1H), 7.00-6.95 (m, | MS m/z calcd for C₂₄H₂₅N₅O₂ 415.2 found 416.1 [M + H]⁺ |
| | N-(2-amino-2-methylpropyl)- | 1H), 5.14 (s, 2H), 3.60 (d, | |
| | 6-(5-(benzyloxy)-1H-indol-2- | J = 6.7 Hz, 2H), 1.33 (s, 6H). | |
| | yl)pyrazine-2-carboxamide | | |
| 23 | 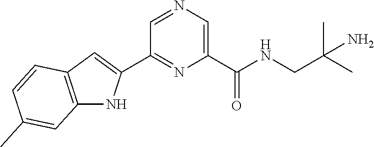 [SMILES: Cc1ccc2cc(-c3cncc(C(=O)NCC(C)(C)N)n3)[nH]c2c1] | ¹H NMR (500 MHz, DMSO- d₆) δ 12.21 (s, 1H), 9.75 (t, J = 6.5 Hz, 1H), 9.47 (s, 1H), 9.00 (s, 1H), 8.11 (s, 3H), 7.54 (d, J = 8.1 Hz, 1H), 7.45 (s, 1H), 7.35 (s, 1H), 6.92 (d, J = 8.2 Hz, 1H), 3.61 (d, J = 6.6 Hz, 2H), 2.44 (s, 3H), 1.33 (s, | MS m/z calcd for C₁₈H₂₁N₅O 323.2 found 324.0 [M + H]⁺ |
| | N-(2-amino-2-methylpropyl)- | 6H). | |
| | 6-(6-methyl-1H-indol-2- | | |
| | yl)pyrazine-2-carboxamide | | |
| 24 | 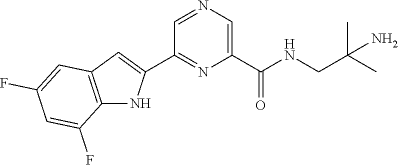 [SMILES: CC(C)(N)CNC(=O)c1cncc(-c2cc3cc(F)cc(F)c3[nH]2)n1] | ¹H NMR (400 MHz, DMSO- d₆) δ 12.49 (s, 1H), 9.80 (t, J = 6.7 Hz, 1H), 9.57 (s, 1H), 9.09 (s, 1H), 8.15 (s, 3H), 7.63 (t, J = 2.6 Hz, 1H), 7.35 (dd, J = 9.2, 2.2 Hz, 1H), 7.20-7.10 (m, 1H), 3.60 (d, J = 6.6 Hz, 2H), 1.32 (s, 6H). ¹⁹F NMR (376 MHz, DMSO- | MS m/z calcd for C₁₇H₁₇F₂N₅O 345.1 found 346.2 [M + H]⁺ |
| | N-(2-amino-2-methylpropyl)- | d₆) δ -121.1, -126.9 | |
| | 6-(5,7-difluoro-1H-indol-2- | | |
| | yl)pyrazine-2-carboxamide | | |
| 25 | 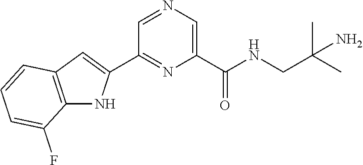 [SMILES: CC(C)(N)CNC(=O)c1cncc(-c2cc3cccc(F)c3[nH]2)n1] | ¹H NMR (400 MHz, DMSO- d₆) δ 12.30 (s, 1H), 9.77 (t, J = 6.7 Hz, 1H), 9.56 (s, 1H), 9.07 (s, 1H), 8.10 (s, 3H), 7.65 (t, J = 2.6 Hz, 1H), 7.50 (d, J = 7.1 Hz, 1H), 7.13-7.02 (m, 2H), 3.60 (d, J = 6.6 Hz, 2H), 1.33 (s, 6H). ¹⁹F NMR (376 MHz, DMSO- | MS m/z calcd for C₁₇H₁₈FN₅O 327.2 found 328.1 [M + H]⁺ |
| | N-(2-amino-2-methylpropyl)- | d₆) δ -131.0 | |
| | 6-(7-fluoro-1H-indol-2- | | |
| | yl)pyrazine-2-carboxamide | | |
| 26 | 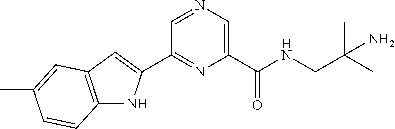 [SMILES: Cc1ccc2[nH]c(-c3cncc(C(=O)NCC(C)(C)N)n3)cc2c1] | ¹H NMR (500 MHz, DMSO- d₆) δ 12.23 (s, 1H), 9.73 (t, J = 6.7 Hz, 1H), 9.48 (s, 1H), 9.01 (d, J = 1.6 Hz, 1H), 8.10 (s, 3H), 7.45 (d, J = 9.3 Hz, 1H), 7.43 (s, 1H), 7.42 (s, 1H), 7.07 (d, J = 8.3 Hz, 1H), 3.61 (d, J = 6.5 Hz, | MS m/z calcd for C₁₈H₂₁N₅O 323.2 found 324.0 [M + H]⁺ |
| | N-(2-amino-2-methylpropyl)- | 2H), 2.40 (s, 3H), 1.33 (s, | |
| | 6-(5-methyl-1H-indol-2- | 6H). | |
| | yl)pyrazine-2-carboxamide | | |
| 27 | 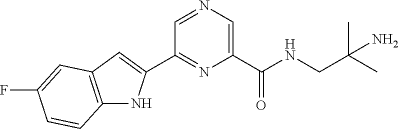 [SMILES: CC(C)(N)CNC(=O)c1cncc(-c2cc3cc(F)ccc3[nH]2)n1] | ¹H NMR (500 MHz, DMSO- d₆) δ 12.53 (s, 1H), 9.74 (t, J = 6.7 Hz, 1H), 9.52 (s, 1H), 9.06 (s, 1H), 8.09 (s, 3H), 7.56 (dd, J = 9.1, 4.5 Hz, 1H), 7.50 (s, 1H), 7.44 (dd, J = 9.8, 2.6 Hz, 1H), 7.10 (td, J = 9.1, 2.5 Hz, | MS m/z calcd for C₁₇H₁₈FN₅O 327.2 found 328.1 [M + H]⁺ |
| | N-(2-amino-2-methylpropyl)- | 1H), 3.61 (d, J = 6.5 Hz, | |
| | 6-(5-fluoro-1H-indol-2- | 2H), 1.33 (s, 6H). | |
| | yl)pyrazine-2-carboxamide | ¹⁹F NMR (470 MHz, DMSO- | |
| | | d₆) δ -123.7 | |
| 28 | 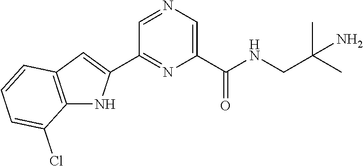 [SMILES: CC(C)(N)CNC(=O)c1cncc(-c2cc3cccc(Cl)c3[nH]2)n1] | ¹H NMR (300 MHz, DMSO- d₆) δ 11.83 (s, 1H), 9.74 (t, J = 6.6 Hz, 1H), 9.58 (s, 1H), 9.06 (s, 1H), 8.04 (s, 3H), 7.69 (s, 1H), 7.65 (d, J = 7.8 Hz, 1H), 7.31 (d, J = 7.5 Hz, 1H), 7.09 (t, J = 7.6 Hz, 1H), 3.58 (d, J = 6.5 Hz, | MS m/z calcd for C₁₇H₁₈ClN₅O 343.1 found 344.2 [M + H]⁺ |
| | N-(2-amino-2-methylpropyl)- | 3H), 1.31 (s, 6H). | |
| | 6-(7-chloro-1H-indol-2- | | |
| | yl)pyrazine-2-carboxamide | | |
| 29 | 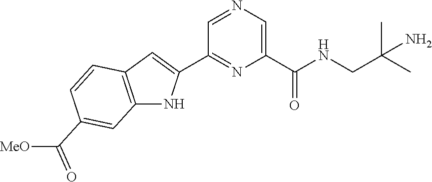 [SMILES: COC(=O)c1ccc2cc(-c3cncc(C(=O)NCC(C)(C)N)n3)[nH]c2c1] | ¹H NMR (500 MHz, DMSO- d₆) δ 12.89 (s, 1H), 9.79 (t, J = 6.6 Hz, 1H), 9.58 (s, 1H), 9.10 (s, 1H), 8.24 (s, 1H), 8.07 (s, 3H), 7.77 (d, J = 8.4 Hz, 1H), 7.69 (dd, J = 8.4, 1.5 Hz, 1H), 7.61 (dd, J = 2.2, 0.9 Hz, 1H), 3.90 (s, 3H), 3.62 (d, J = 6.6 Hz, | MS m/z calcd for C₁₉H₂₁N₅O₃ 367.2 found 368.2 [M + H]⁺ |
| | Methyl 2-(6-((2-amino-2- | 2H), 1.34 (s, 6H). | |
| | methylpropyl)carbamoyl)pyrazin- | | |
| | 2-yl)-1H-indole-6- | | |
| | carboxylate | | |
| 30 | 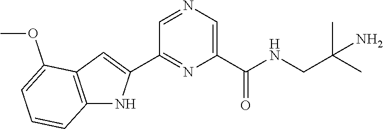 [SMILES: COc1cccc2[nH]c(-c3cncc(C(=O)NCC(C)(C)N)n3)cc12] | ¹H NMR (500 MHz, DMSO- d₆) δ 12.32 (s, 1H), 9.72 (t, J = 6.7 Hz, 1H), 9.52 (s, 1H), 9.00 (s, 1H), 8.09 (s, 3H), 7.57 (d, J = 2.4 Hz, 1H), 7.16 (d, J = 4.1 Hz, 2H), 6.57 (d, J = 4.3 Hz, 1H), 3.92 (s, 3H), 3.60 (d, | MS m/z calcd for C₁₈H₂₁N₅O₂ 339.2 found 340.1[M + H]⁺ |
| | N-(2-amino-2-methylpropyl)- | J = 6.6 Hz, 2H), 1.33 (s, 6H). | |
| | 6-(4-methoxy-1H-indol-2- | | |
| | yl)pyrazine-2-carboxamide | | |
| 31 | 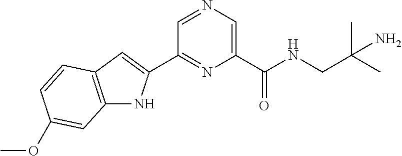 [SMILES: COc1ccc2cc(-c3cncc(C(=O)NCC(C)(C)N)n3)[nH]c2c1] | ¹H NMR (500 MHz, DMSO- d₆) δ 12.15 (s, 1H), 9.68 (t, J = 6.5 Hz, 1H), 9.43 (s, 1H), 8.97 (s, 1H), 8.07 (s, 3H), 7.54 (d, J = 8.7 Hz, 1H), 7.44 (s, 1H), 7.01 (s, 1H), 6.75 (dd, J = 8.8, 2.6 Hz, 1H), 3.83 (s, 3H), 3.60 (d, J = 6.6 Hz, 2H), 1.34 (s, | MS m/z calcd for C₁₈H₂₁N₅O₂ 339.2 found 340.1 [M + H]⁺ |
| | N-(2-amino-2-methylpropyl)- | 6H). | |
| | 6-(6-methoxy-1H-indol-2- | | |
| | yl)pyrazine-2-carboxamide | | |
| 32 | 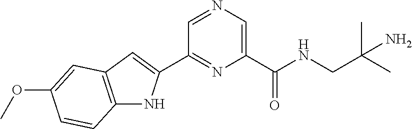 [SMILES: COc1ccc2[nH]c(-c3cncc(C(=O)NCC(C)(C)N)n3)cc2c1] | ¹H NMR (500 MHz, DMSO- d₆) δ 12.22 (s, 1H), 9.71 (t, J = 6.5 Hz, 1H), 9.47 (s, 1H), 9.01 (s, 1H), 8.10 (s, 3H), 7.45 (d, J = 8.8 Hz, 1H), 7.41 (s, 1H), 7.13 (s, 1H), 6.91-6.87 (m, 1H), | MS m/z calcd for C₁₈H₂₁N₅O₂ 339.2 found 340.1 [M + H]⁺ |
| | N-(2-amino-2-methylpropyl)- | 3.80 (s, 3H), 3.60 (d, J = 6.6 | |
| | 6-(5-methoxy-1H-indol-2- | Hz, 2H), 1.33 (s, 6H). | |
| | yl)pyrazine-2-carboxamide | | |
| 33 | 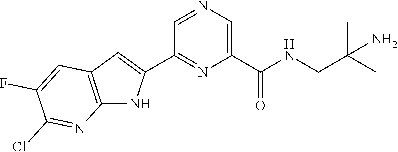 [SMILES: CC(C)(N)CNC(=O)c1cncc(-c2cc3cc(F)c(Cl)nc3[nH]2)n1] | ¹H NMR (300 MHz, DMSO- d₆) δ 12.96 (s, 1H), 9.65 (t, J = 6.9 Hz, 1H), 9.61 (s, 1H), 9.10 (s, 1H), 8.28 (d, J = 9.2 Hz, 1H), 7.97 (s, 3H), 7.58 (d, J = 2.0 Hz, 1H), 3.55 (d, J = 6.6 Hz, 2H), 1.32 (s, 6H). ¹⁹F NMR (282 MHz, DMSO- | MS m/z calcd for C₁₆H₁₆ClFN₆O 362.1 found 363.1 [M + H]⁺ |
| | N-(2-amino-2-methylpropyl)- | d₆) δ -131.0 | |
| | 6-(6-chloro-5-fluoro-1H- | | |
| | pyrrolo[2,3-b]pyridin-2- | | |
| | yl)pyrazine-2-carboxamide | | |
| 34 | 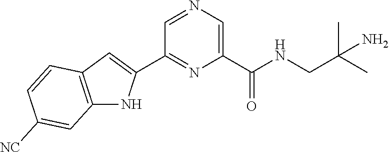 [SMILES: CC(C)(N)CNC(=O)c1cncc(-c2cc3ccc(C#N)cc3[nH]2)n1] | ¹H NMR (500 MHz, DMSO- d₆) δ 12.56 (s, 1H), 9.60 (s, 1H), 9.50 (br, 1H), 9.12 (s, 1H), 8.06 (s, 1H), 7.92- 7.66 (br, 3H), 7.87 (d, J = 8.3 Hz, 1H), 7.69 (s, 1H), 7.43 (d, J = 8.3 Hz, 1H), 3.60 (d, J = 6.7 Hz, 2H), 1.33 (s, 6H). | MS m/z calcd for C₁₈H₁₈N₆O 334.2 found 335.3 [M + H]⁺ |
| | N-(2-amino-2-methylpropyl)- | | |
| | 6-(6-cyano-1H-indol-2- | | |
| | yl)pyrazine-2-carboxamide | | |
| 35 | 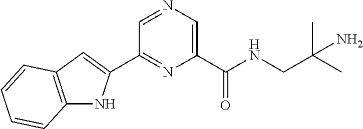 [SMILES: CC(C)(N)CNC(=O)c1cncc(-c2cc3ccccc3[nH]2)n1] | ¹H NMR (500 MHz, DMSO- d₆) δ 12.41 (s, 1H), 9.77 (t, J = 6.6 Hz, 1H), 9.51 (s, 1H), 9.03 (s, 1H), 8.13 (s, 3H), 7.66 (d, J = 8.0 Hz, 1H), 7.57 (dd, J = 8.2, 1.0 Hz, 1H), 7.51 (d, J = 2.1 Hz, 1H), 7.24 (ddd, J = 8.2, 6.9, | MS m/z calcd for C₁₇H₁₉N₅O 309.2 found 310.0 [M + H]⁺ |
| | N-(2-amino-2-methylpropyl)- | 1.2 Hz, 1H), 7.08 (ddd, J = | |
| | 6-(1H-indol-2-yl)pyrazine-2- | 8.0, 6.9, 1.0 Hz, 1H), 3.61 | |
| | carboxamide | (d, J = 6.6 Hz, 2H), 1.34 (s, | |
| | | 6H). | |
| 36 | 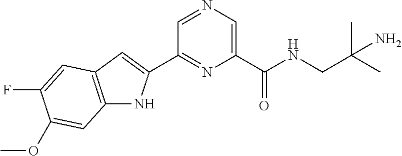 [SMILES: COc1cc2[nH]c(-c3cncc(C(=O)NCC(C)(C)N)n3)cc2cc1F] | ¹H NMR (400 MHz, DMSO- d₆) δ 12.14 (s, 2H), 9.57 (t, J = 6.5 Hz, 1H), 9.44 (s, 1H), 8.99 (s, 1H), 7.96 (s, 3H), 7.47 (d, J = 11.7 Hz, 1H), 7.43 (d, J = 2.1 Hz, 1H), 7.14 (d, J = 7.3 Hz, 1H), 3.58 (d, J = 6.6 Hz, 2H), 1.33 (s, 6H). | MS m/z calcd for C₁₈H₂₀FN₅O₂ 357.2 found 358.1 [M + H]⁺ |
| | N-(2-amino-2-methylpropyl)- | ¹⁹F NMR (376 MHz, DMSO- | |
| | 6-(5-fluoro-6-methoxy-1H- | d₆) δ -142.5 | |
| | indol-2-yl)pyrazine-2- | | |
| | carboxamide | | |
| 37 | 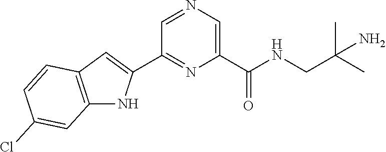 [SMILES: CC(C)(N)CNC(=O)c1cncc(-c2cc3ccc(Cl)cc3[nH]2)n1] | ¹H NMR (400 MHz, DMSO- d₆) δ 12.86 (s, 1H), 9.64 (t, J = 6.8 Hz, 1H), 9.60 (s, 1H), 9.10 (s, 1H), 8.19 (d, J = 8.2 Hz, 1H), 7.91 (s, 3H), 7.60 (s, 1H), 7.24 (d, J = 8.3 Hz, 1H), 3.56 (d, J = 6.9 Hz, 2H), 1.34 (s, 6H). | MS m/z calcd for C₁₆H₁₇ClN₆O 344.1 found 345.3 [M + H]⁺ |
| | N-(2-amino-2-methylpropyl)- | | |
| | 6-(6-chloro-1H-pyrrolo[2,3- | | |
| | b]pyridin-2-yl)pyrazine-2- | | |
| | carboxamide | | |
| 38 | 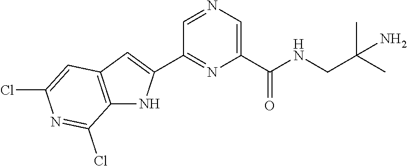 [SMILES: CC(C)(N)CNC(=O)c1cncc(-c2cc3cc(Cl)nc(Cl)c3[nH]2)n1] | ¹H NMR (500 MHz, DMSO- d₆) δ 12.67 (s, 1H), 9.77 (t, J = 6.5 Hz, 1H), 9.69 (s, 1H), 9.19 (s, 1H), 8.04 (s, 3H), 7.85 (s, 1H), 7.73 (d, J = 1.9 Hz, 1H), 3.60 (d, J = 6.6 Hz, 2H), 1.33 (s, 6H). | MS m/z calcd for C₁₆H1₆Cl₂N₆O 378.1 found 379.2 [M + H]⁺ |
| | N-(2-amino-2-methylpropyl)- | | |
| | 6-(5,7-dichloro-1H- | | |
| | pyrrolo[2,3-c]pyridin-2- | | |
| | yl)pyrazine-2-carboxamide | | |
| 39 | 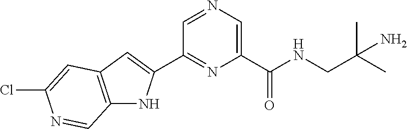 [SMILES: CC(C)(N)CNC(=O)c1cncc(-c2cc3cc(Cl)ncc3[nH]2)n1] | ¹H NMR (500 MHz, DMSO- d₆) δ 12.66 (s, 1H), 9.65 (s, 1H), 9.50 (t, J = 6.7 Hz, 1H), 9.19 (s, 1H), 8.77 (t, J = 1.0 Hz, 1H), 7.94 (s, 3H), 7.79 (d, J = 0.9 Hz, 1H), 7.61 (dd, J = 2.0, 0.9 Hz, 1H), 3.61 (d, J = 6.7 Hz, 2H), | MS m/z calcd for C₁₆H₁₇ClN₆O 344.1 found 345.3 [M + H]⁺ |
| | N-(2-amino-2-methylpropyl)- | 1.34 (s, 6H). | |
| | 6-(5-chloro-1H-pyrrolo[2,3- | | |
| | c]pyridin-2-yl)pyrazine-2- | | |
| | carboxamide | | |
| 40 | 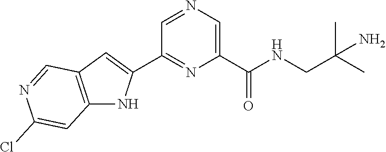 [SMILES: CC(C)(N)CNC(=O)c1cncc(-c2cc3cnc(Cl)cc3[nH]2)n1] | ¹H NMR (500 MHz, DMSO- d₆) δ 12.55 (s, 1H), 9.63 (s, 1H), 9.46 (t, J = 6.7 Hz, 1H), 9.14 (s, 1H), 8.83 (d, J = 0.9 Hz, 1H), 7.88 (s, 3H), 7.76 (dd, J = 2.0, 1.0 Hz, 1H), 7.63-7.58 (m, 1H), 3.60 (d, J = 6.7 Hz, 2H), 1.34 (s, 6H). | MS m/z calcd for C₁₆H₁₇ClN₆O 344.1 found 345.1 [M + H]⁺ |
| | N-(2-amino-2-methylpropyl)- | | |
| | 6-(6-chloro-1H-pyrrolo[3,2- | | |
| | c]pyridin-2-yl)pyrazine-2- | | |
| | carboxamide | | |
| 41 | 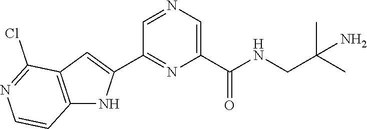 [SMILES: CC(C)(N)CNC(=O)c1cncc(-c2cc3c(Cl)nccc3[nH]2)n1] | ¹H NMR (400 MHz, DMSO- d₆) δ 12.74 (s, 1H), 9.69 (s, 1H), 9.46 (t, J = 6.6 Hz, 1H), 9.14 (s, 1H), 8.11 (d, J = 5.7 Hz, 1H), 7.89 (s, 3H), 7.75 (dd, J = 2.2, 1.0 Hz, 1H), 7.59 (dd, J = 5.7, 1.0 Hz, 1H), 3.59 (d, J = 6.6 Hz, | MS m/z calcd for C₁₆H₁₇ClN₆O 344.1 found 345.3 [M + H]⁺ |
| | N-(2-amino-2-methylpropyl)- | 2H), 1.33 (s, 6H). | |
| | 6-(4-chloro-1H-pyrrolo[3,2- | | |
| | c]pyridin-2-yl)pyrazine-2- | | |
| | carboxamide | | |
| 42 | 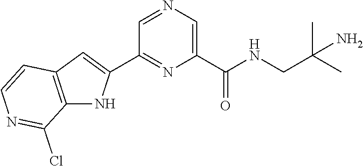 [SMILES: CC(C)(N)CNC(=O)c1cncc(-c2cc3ccnc(Cl)c3[nH]2)n1] | ¹H NMR (300 MHz, DMSO- d₆) δ 12.37 (s, 1H), 9.72 (t, J = 6.6 Hz, 1H), 9.67 (s, 1H), 9.15 (s, 1H), 8.00 (s, 3H), 7.98 (s, 1H), 7.75 (d, J = 2.2 Hz, 1H), 7.69 (d, J = 5.7 Hz, 1H), 3.59 (d, J = 6.4 Hz, 2H), 1.31 (s, 6H). | MS m/z calcd for C₁₆H₁₇ClN₆O 344.1 found 345.1 [M + H]⁺ |
| | N-(2-amino-2-methylpropyl)- | | |
| | 6-(7-chloro-1H-pyrrolo[2,3- | | |
| | c]pyridin-2-yl)pyrazine-2- | | |
| | carboxamide | | |
| 43 | 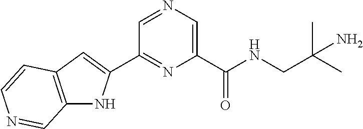 [SMILES: CC(C)(N)CNC(=O)c1cncc(-c2cc3ccncc3[nH]2)n1] | ¹H NMR (500 MHz, DMSO- d₆) δ 14.78 (s, 1H), 10.01 (t, J = 6.5 Hz, 1H), 9.81 (s, 1H), 9.29 (s, 1H), 9.17 (s, 1H), 8.37 (d, J = 6.4 Hz, 1H), 8.29 (d, J = 6.4 Hz, 1H), 8.11 (s, 3H), 7.94 (d, J = 1.8 Hz, 1H), 3.63 (d, J = | MS m/z calcd for C₁₆H₁₈N₆O 310.2 found 311.0 [M + H]⁺ |
| | N-(2-amino-2-methylpropyl)- | 6.6 Hz, 2H), 1.35 (s, 6H). | |
| | 6-(1H-pyrrolo[2,3-c]pyridin-2- | | |
| | yl)pyrazine-2-carboxamide | | |
| 44 | 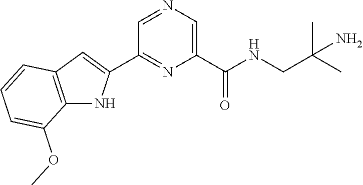 [SMILES: COc1cccc2cc(-c3cncc(C(=O)NCC(C)(C)N)n3)[nH]c12] | ¹H NMR (500 MHz, DMSO- d₆) δ 11.75 (s, 1H), 9.71 (t, J = 6.7 Hz, 1H), 9.53 (s, 1H), 9.03 (s, 1H), 7.99 (s, 3H), 7.56 (d, J = 2.4 Hz, 1H), 7.25 (d, J = 8.0 Hz, 1H), 7.03 (t, J = 7.8 Hz, 1H), 6.83 (d, J = 7.7 Hz, 1H), 4.00 (s, 3H), 3.58 (d, J = 6.6 Hz, 2H), 1.34 (s, 6H). | MS m/z calcd for C₁₈H₂₁N₅O₂ 339.2 found 340.1 [M+H]+ |
| | N-(2-amino-2-methylpropyl)- | | |
| | 6-(7-methoxy-1H-indol-2- | | |
| | yl)pyrazine-2-carboxamide | | |
| 45 | 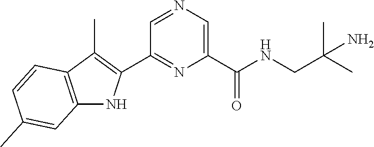 [SMILES: Cc1ccc2c(C)c(-c3cncc(C(=O)NCC(C)(C)N)n3)[nH]c2c1] | ¹H NMR (500 MHz, DMSO- d₆) δ 11.90 (s, 1H), 9.66 (t, J = 6.6 Hz, 1H), 9.29 (s, 1H), 9.02 (s, 1H), 8.10 (s, 3H), 7.57 (d, J = 8.2 Hz, 1H), 7.32 (s, 1H), 6.92 (dd, J= 8.3, 1.5 Hz, 1H), 3.61 (d, J = 6.6 Hz, 2H), 2.66 (s, 3H), 2.44 (s, 3H), 1.33 (s, 6H) | MS m/z calcd for C₁₉H₂₃N₅O 337.2 found 338.2 [M + H]⁺ |
| | N-(2-Amino-2-methylpropyl)- | | |
| | 6-(3,6-dimethyl-1H-indol-2- | | |
| | yl)pyrazine-2-carboxamide | | |
| 46 | 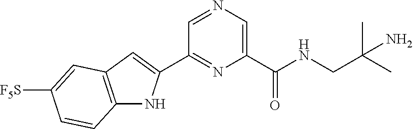 | ¹H NMR (500 MHz, DMSO- d₆) δ 12.95 (s, 1H), 9.75 (t, J = 6.6 Hz, 1H), 9.59 (s, 1H), 9.11 (s, 1H), 8.30 (s, 1H), 8.05 (s, 3H), 7.73 (d, J = 1.4 Hz, 2H), 7.70 (d, J = 2.1 Hz, 1H), 3.61 (d, J = 6.6 Hz, 2H), 1.34 (s, 6H). | MS m/z calcd for C₁₇H₁₈F₅N₅OS 435.1 found 436.1 [M + H]⁺ |
| | N-(2-amino-2-methylpropyl)- | | |
| | 6-(5-(pentafluoro-λ⁶- | | |
| | sulfaneyl)-1H-indol-2- | | |
| | yl)pyrazine-2-carboxamide | | |
| 47 | 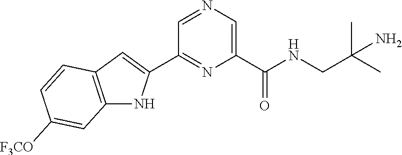 [SMILES: CC(C)(N)CNC(=O)c1cncc(-c2cc3ccc(OC(F)(F)F)cc3[nH]2)n1] | ¹H NMR (400 MHz, DMSO- d₆) δ 12.3 (s, 1H), 9.56 (s, 1H), 9.50 (t, J = 6.7 Hz, 1H), 9.09 (s, 1H), 7.92 (s, 3H), 7.80 (d, J = 8.6 Hz, 1H), 7.64 (dd, J = 2.1, 0.9 Hz, 1H), 7.52 (s, 1H), 7.10 (ddd, J = 8.7, 2.2, 1.0 Hz, 1H), 3.60 (d, J = 6.7 Hz, 2H), | MS m/z calcd for C₁₈H₁₈F₃N₅O₂ 393.1 found 394.3 [M + H]⁺ |
| | N-(2-amino-2-methylpropyl)- | 1.34 (s, 6H). | |
| | 6-(6-(trifluoromethoxy)-1H- | ¹⁹F NMR (376 MHz, DMSO- | |
| | indol-2-yl)pyrazine-2- | d₆) δ -56.8, -73.5 (TFA) | |
| | carboxamide | | |
| 48 | 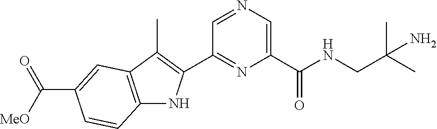 [SMILES: COC(=O)c1ccc2[nH]c(-c3cncc(C(=O)NCC(C)(C)N)n3)c(C)c2c1] | ¹H NMR (500 MHz, DMSO- d₆) δ 12.59 (s, 1H), 9.73 (t, J = 6.7 Hz, 1H), 9.39 (s, 1H), 9.13 (s, 1H), 8.22 (s, 1H), 8.08 (s, 3H), 7.81 (d, J = 8.5 Hz, 1H), 7.68 (dd, J = 8.4, 1.5 Hz, 1H), 3.89 (s, 3H), 3.62 (d, J = 6.6 Hz, | MS m/z calcd for C₂₀H₂₃N₅O3 381.2 found 382.0 [M + H]⁺ |
| | Methyl 2-(6-((2-amino-2- | 2H), 2.72 (s, 3H), 1.34 (s, | |
| | methylpropyl)carbamoyl)pyrazin- | 6H). | |
| | 2-yl)-3-methyl-1H-indole- | | |
| | 5-carboxylate | | |
| 49 | 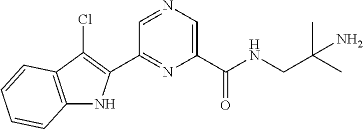 [SMILES: CC(C)(N)CNC(=O)c1cncc(-c2[nH]c3ccccc3c2Cl)n1] | ¹H NMR (500 MHz, DMSO- d₆) δ 12.82 (s, 1H), 9.87 (t, J = 6.6 Hz, 1H), 9.75 (s, 1H), 9.15 (s, 1H), 8.12 (s, 3H), 7.67 (dd, J = 8.2, 0.9 Hz, 1H), 7.64 (dd, J = 8.0, 0.9 Hz, 1H), 7.35 (ddd, J = 8.3, 7.0, 1.2 Hz, 1H), 7.21 | MS m/z calcd for C₁₇H₁₈ClN₅O 343.1 found 344.0 [M + H]⁺ |
| | N-(2-amino-2-methylpropyl)- | (ddd, J = 7.9, 6.9, 0.9 Hz, | |
| | 6-(3-chloro-1H-indol-2- | 1H), 3.62 (d, J = 6.6 Hz, | |
| | yl)pyrazine-2-carboxamide | 2H), 1.34 (s, 6H). | |
| 50 | 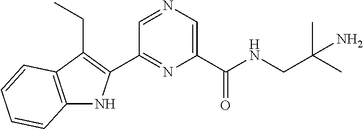 [SMILES: CCc1c(-c2cncc(C(=O)NCC(C)(C)N)n2)[nH]c2ccccc12] | ¹H NMR (500 MHz, DMSO- d₆) δ 12.00 (s, 1H), 9.57 (s, 1H), 9.26 (d, J = 2.4 Hz, 1H), 9.06 (d, J = 2.1 Hz, 1H), 8.08 (s, 3H), 7.73- 7.66 (m, 1H), 7.54 (d, J = 8.3 Hz, 1H), 7.24 (t, J = 7.8 Hz, 1H), 7.08 (t, J = 7.3 Hz, | MS m/z calcd for C₁₉H₂₃N₅O 337.2 found 338.2 [M+H]+ |
| | N-(2-amino-2-methylpropyl)- | 1H), 3.67-3.54 (m, 2H), | |
| | 6-(3-ethyl-1H-indol-2- | 3.19 (d, J = 7.9 Hz, 2H), | |
| | yl)pyrazine-2-carboxamide | 1.33 (s, 6H), 1.29 (t, J = 7.9 | |
| | | Hz, 3H). | |
| 51 | 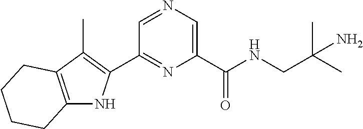 [SMILES: Cc1c(-c2cncc(C(=O)NCC(C)(C)N)n2)[nH]c2c1CCCC2] | ¹H NMR (500 MHz, DMSO- d₆) δ 11.30 (s, 1H), 9.40 (t, J = 6.7 Hz, 1H), 8.93 (s, 1H), 8.76 (s, 1H), 7.99 (s, 3H), 3.55 (d, J = 6.6 Hz, 2H), 2.68-2.64 (m, 2H), 2.42-2.36 (m, 2H), 2.28 (s, 3H), 1.80-1.71 (m, 4H), | MS m/z calcd for C₁₈H₂₅N₅O 327.2 found 328.1 [M + H]⁺ |
| | N-(2-amino-2-methylpropyl)- | 1.30 (s, 6H). | |
| | 6-(3-methyl-4,5,6,7- | | |
| | tetrahydro-1H-indol-2- | | |
| | yl)pyrazine-2-carboxamide | | |
| 52 | 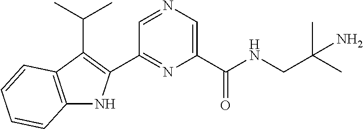 [SMILES: CC(C)c1c(-c2cncc(C(=O)NCC(C)(C)N)n2)[nH]c2ccccc12] | ¹HNMR (500 MHz, DMSO- d₆) δ 11.54 (s, 1H), 9.19 (s, 1H), 9.12 (t, J = 6.7 Hz, 1H), 9.09 (s, 1H), 7.89- 7.84 (m, 3H), 7.49 (d, J = 8.2 Hz, 1H), 7.22 (ddd, J = 8.1, 6.8, 1.0 Hz, 1H), 7.08- 7.01 (m, 1H), 3.80 (h, J = | MS m/z calcd for C₂₀H₂₅N₅O 351.2 found 352.3 [M + H]⁺ |
| | N-(2-amino-2-methylpropyl)- | 7.0 Hz, 1H), 3.57 (d, J = 6.7 | |
| | 6-(3-isopropyl-1H-indol-2- | Hz, 2H), 1.51 (d, J = 7.0 Hz, | |
| | yl)pyrazine-2-carboxamide | 6H), 1.31 (s, 6H). | |
| 53 | 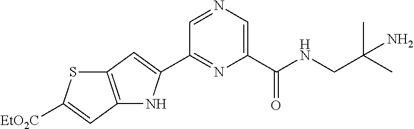 [SMILES: CCOC(=O)c1cc2[nH]c(-c3cncc(C(=O)NCC(C)(C)N)n3)cc2s1] | ¹H NMR (500 MHz, DMSO- d₆) δ 12.89 (s, 1H), 9.66 (t, J = 6.8 Hz, 1H), 9.42 (s, 1H), 9.01 (s, 1H), 8.04 (s, 3H), 7.77 (s, 1H), 7.55 (s, 1H), 4.32 (q, J = 7.1 Hz, 2H), 3.59 (d, J = 6.6 Hz, 2H), 1.33 (d, J = 3.4 Hz, | MS m/z calcd for C₁₈H₂₁N₅O₃S 387.1 found 388.0 [M + H]⁺ |
| | Ethyl 5-(6-((2-amino-2- | 9H). | |
| | methylpropyl)carbamoyl)pyrazin- | | |
| | 2-yl)-4H-thieno[3,2- | | |
| | b]pyrrole-2-carboxylate | | |
| 54 | 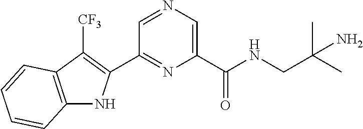 [SMILES: CC(C)(N)CNC(=O)c1cncc(-c2[nH]c3ccccc3c2C(F)(F)F)n1] | ¹H NMR (500 MHz, DMSO- d₆) δ 13.18 (s, 1H), 9.47 (t, J = 6.7 Hz, 1H), 9.27 (s, 1H), 9.26 (s, 1H), 8.03 (s, 3H), 7.79 (d, J = 8.2 Hz, 1H), 7.74 (d, J = 8.2 Hz, 1H), 7.40 (t, J = 7.6 Hz, 1H), 7.29 (t, J= 7.6 Hz, 1H), 3.60 | MS m/z calcd for C₁₈H₁₈F₃N₅O 377.2 found 378.0 [M + H]⁺ |
| | N-(2-amino-2-methylpropyl)- | (d, J = 6.7 Hz, 2H), 1.32 (s, | |
| | 6-(3-(trifluoromethyl)-1H- | 6H). | |
| | indol-2-yl)pyrazine-2- | ¹⁹F NMR (470 MHz, DMSO- | |
| | carboxamide | d₆) δ -51.2 | |
| 55 | 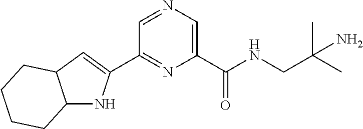 [SMILES: CC(C)(N)CNC(=O)c1cncc(C2=CC3CCCCC3N2)n1] | ¹H NMR (500 MHz, DMSO- d₆) δ 11.56 (s, 1H), 9.51 (t, J = 6.8 Hz, 1H), 9.06 (s, 1H), 8.74 (s, 1H), 8.02 (s, 3H), 6.83 (d, J = 2.2 Hz, 1H), 3.54 (d, J = 6.6 Hz, 2H), 2.68 (t, J = 6.2 Hz, 2H), 2.49 (t, J = 5.4 Hz, 2H), 1.83- | MS m/z calcd for C₁₇H₂₃N₅O 313.2 found 314.1 [M + H]⁺ |
| | N-(2-amino-2-methylpropyl)- | 1.75 (m, 2H), 1.74-1.69 | |
| | 6-(4,5,6,7-tetrahydro-1H- | (m, 2H), 1.30 (s, 6H). | |
| | indol-2-yl)pyrazine-2- | | |
| | carboxamide | | |
| 56 | 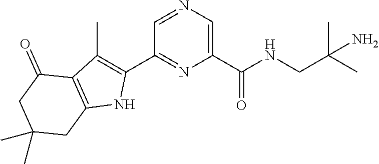 [SMILES: Cc1c(-c2cncc(C(=O)NCC(C)(C)N)n2)[nH]c2c1C(=O)CC(C)(C)C2] | ¹H NMR (500 MHz, DMSO- d₆) δ 12.35 (s, 1H), 9.57 (t, J = 6.6 Hz, 1H), 9.07 (s, 1H), 8.94 (s, 1H), 8.00 (s, 3H), 3.57 (d, J = 6.4 Hz, 2H), 2.82 (s, 2H), 2.66 (s, 3H), 2.29 (s, 2H), 1.30 (s, 6H), 1.08 (s, 6H). | MS m/z calcd for C₂₀H₂₇N₅O₂ 369.2 found 370.3 [M + H]⁺ |
| | N-(2-amino-2-methylpropyl)- | | |
| | 6-(3,6,6-trimethyl-4-oxo- | | |
| | 4,5,6,7-tetrahydro-1H-indol-2- | | |
| | yl)pyrazine-2-carboxamide | | |
| 57 | 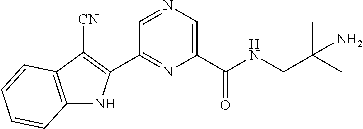 [SMILES: CC(C)(N)CNC(=O)c1cncc(-c2[nH]c3ccccc3c2C#N)n1] | ¹H NMR (500 MHz, DMSO- d₆) δ 13.15 (s, 1H), 9.61 (s, 1H), 9.26 (s, 1H), 9.00 (t, J = 6.8 Hz, 1H), 7.90 (s, 3H), 7.78 (d, J = 8.0 Hz, 1H), 7.72 (dd, J = 8.3, 1.0 Hz, 1H), 7.46 (ddd, J = 8.2, 7.0, 1.2 Hz, 1H), 7.36 (ddd, J = | MS m/z calcd for C₁₈H₁₈N₆O 334.2 found 335.2 [M + H]⁺ |
| | N-(2-amino-2-methylpropyl)- | 8.0, 7.0, 1.0 Hz, 1H), 3.60 | |
| | 6-(3-cyano-1H-indol-2- | (d, J = 6.8 Hz, 2H), 1.33 (s, | |
| | yl)pyrazine-2-carboxamide | 6H). | |
| 58 | 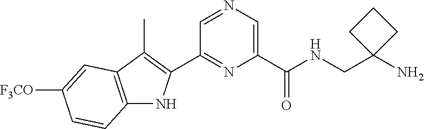 [SMILES: Cc1c(-c2cncc(C(=O)NCC3(N)CCC3)n2)[nH]c2ccc(OC(F)(F)F)cc12] | ¹H NMR (300 MHz, DMSO- d₆) δ 12.37 (s, 1H), 9.77 (t, J = 6.0 Hz, 1H), 9.36 (s, 1H), 9.09 (s, 1H), 8.28 (s, 3H), 7.70 (s, 1H), 7.61 (d, J = 8.7 Hz, 1H), 7.21 (d, J = 8.5 Hz, 1H), 3.80 (d, J = 6.3 Hz, 2H), 2.68 (s, 3H), 1.95- | MS m/z calcd for C₂₀H₂₀F₃N₅O₂ 419.2 found 420.3 [M + H]⁺ |
| | N-((1- | 1.85 (m, 4H), 1.26-1.20 | |
| | aminocyclobutyl)methyl)-6-(3- | (m, 2H). ¹⁹F NMR (282 | |
| | methyl-5-(trifluoromethoxy)- | MHz, DMSO-d₆) δ -56.9 | |
| | 1H-indol-2-yl)pyrazine-2- | | |
| | carboxamide | | |
| 59 | 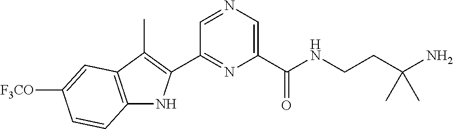 [SMILES: Cc1c(-c2cncc(C(=O)NCCC(C)(C)N)n2)[nH]c2ccc(OC(F)(F)F)cc12] | ¹H NMR (400 MHz, DMSO- d₆) δ 12.19 (s, 1H), 9.56 (t, J = 6.1 Hz, 1H), 9.31 (s, 1H), 9.04 (s, 1H), 8.04 (s, 3H), 7.68 (s, 1H), 7.58 (d, J = 8.8 Hz, 1H), 7.19 (d, J = 8.8 Hz, 1H), 3.50 (q, J = 7.2 Hz, 2H), 2.66 (s, 3H), 1.92 | MS m/z calcd for C₂₀H₂₂F₃N₅O₂ 421.1 found 422.2 [M + H]⁺ |
| | N-(3-amino-3-methylbutyl)-6- | (t, J = 7.6 Hz, 2H), 1.32 (s, | |
| | (3-methyl-5- | 6H). | |
| | (trifluoromethoxy)-1H-indol-2- | ¹⁹F NMR (376 MHz, DMSO- | |
| | yl)pyrazine-2-carboxamide | d₆) δ -56.9 | |
| 60 | 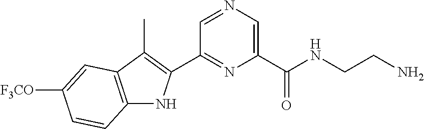 [SMILES: Cc1c(-c2cncc(C(=O)NCCN)n2)[nH]c2ccc(OC(F)(F)F)cc12] | ¹H NMR (400 MHz, DMSO- d₆) δ 12.49 (s, 1H), 9.81 (t, J = 6.1 Hz, 1H), 9.34 (s, 1H), 9.06 (s, 1H), 8.08 (s, 3H), 7.68 (d, J = 2.2 Hz, 1H), 7.62 (d, J = 9.0 Hz, 1H), 7.20 (dd, J = 8.7, 2.2 Hz, 1H), 3.69 (q, J = 6.1 Hz, | MS m/z calcd for C₁₇H₁₆F₃N₅O₂ 379.1 found 380.1 [M + H]⁺ |
| | N-(2-aminoethyl)-6-(3-methyl- | 2H), 3.12 (q, J = 6.0 Hz, | |
| | 5-(trifluoromethoxy)-1H-indol- | 2H), 2.68 (s, 3H). | |
| | 2-yl)pyrazine-2-carboxamide | ¹⁹F NMR (376 MHz, DMSO- | |
| | | d₆) δ -56.9 | |
| 61 | 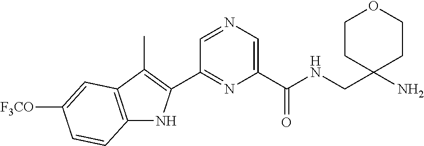 [SMILES: Cc1c(-c2cncc(C(=O)NCC3(N)CCOCC3)n2)[nH]c2ccc(OC(F)(F)F)cc12] | ¹H NMR (500 MHz, DMSO- d₆) δ 11.89 (s, 1H), 9.39 (t, J = 6.7 Hz, 1H), 9.37 (s, 1H), 9.12 (s, 1H), 8.08 (s, 3H), 7.73 (d, J = 2.3 Hz, 1H), 7.58 (d, J = 8.8 Hz, 1H), 7.27-7.21 (m, 1H), 3.83-3.76 (m, 6H), 3.75- 3.69 (m, 4H), 2.70 (s, 3H), | MS m/z calcd for C₂₁H₂₂F₃N₅O₃ 449.2 found 450.1 [M + H]⁺ |
| | N-((4-aminotetrahydro-2H- | 1.89-1.83 (m, 2H), 1.79- | |
| | pyran-4-yl)methyl)-6-(3- | 1.73 (m, 2H). | |
| | methyl-5-(trifluoromethoxy)- | ¹⁹F NMR (376 MHz, DMSO- | |
| | 1H-indol-2-yl)pyrazine-2- | d₆) δ -56.9, -74.0 (TFA) | |
| | carboxamide | | |
| 62 | 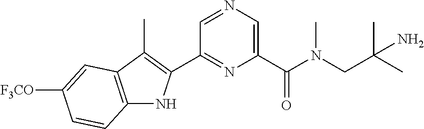 [SMILES: Cc1c(-c2cncc(C(=O)N(C)CC(C)(C)N)n2)[nH]c2ccc(OC(F)(F)F)cc12] | ¹H NMR (400 MHz, DMSO- d₆) δ 11.85 (s, 1H), 9.22 (s, 1H), 8.84 (s, 1H), 8.07 (s, 3H), 7.65 (d, J = 2.2 Hz, 1H), 7.57 (d, J = 8.9 Hz, 1H), 7.19 (dd, J = 8.7, 2.3 Hz, 1H), 3.72 (s, 3H), 3.20 (s, 3H), 2.64 (s, 3H), 1.38 | MS m/z calcd for C₂₀H₂₂F₃N₅O₂ 421.2 found 422.2 [M + H]⁺ |
| | N-(2-amino-2-methylpropyl)- | (s, 6H). | |
| | N-methyl-6-(3-methyl-5- | ¹⁹F NMR (376 MHz, DMSO- | |
| | (trifluoromethoxy)-1H-indol-2- | d₆) δ -56.9 | |
| | yl)pyrazine-2-carboxamide | | |
| 63 | 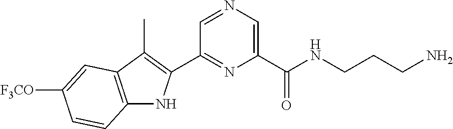 [SMILES: Cc1c(-c2cncc(C(=O)NCCCN)n2)[nH]c2ccc(OC(F)(F)F)cc12] | ¹H NMR (400 MHz, DMSO- d₆) δ 12.18 (s, 1H), 9.58 (t, J = 6.3 Hz, 1H), 9.34 (s, 1H), 9.06 (s, 1H), 7.84 (s, 3H), 7.70 (s, 1H), 7.60 (d, J = 8.8 Hz, 1H), 7.22 (d, J = 9.0 Hz, 1H), 3.51 (q, J = 6.5 Hz, 2H), 2.91 (q, J = 6.5 Hz, | MS m/z calcd for C₁₈H₁₈F₃N₅O₂ 393.1 found 392.1 [M + H]⁺ |
| | N-(3-aminopropyl)-6-(3- | 2H), 2.68 (s, 3H), 1.95- | |
| | methyl-5-(trifluoromethoxy)- | 1.88 (q, J = 7.0 Hz, 2H). | |
| | 1H-indol-2-yl)pyrazine-2- | ¹⁹F NMR (376 MHz, DMSO- | |
| | carboxamide | d₆) δ -56.9 | |
| 64 | 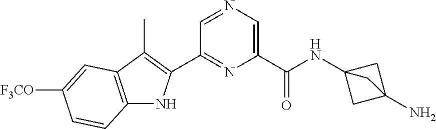 [SMILES: Cc1c(-c2cncc(C(=O)NC34CC(N)(C3)C4)n2)[nH]c2ccc(OC(F)(F)F)cc12] | ¹H NMR (400 MHz, DMSO- d₆) δ 12.19 (s, 1H), 9.89 (s, 1H), 9.33 (s, 1H), 9.03 (s, 1H), 8.85 (s, 4H), 7.70 (s, 1H), 7.61 (d, J = 8.7 Hz, 1H), 7.21 (d, J = 8.9 Hz, 1H), 2.67 (s, 3H), 2.45 (s, 6H) | MS m/z calcd for C₂₀H₁₈F₃N₅O₂ 417.1 found 418.2 [M + H]⁺ |
| | N-(3 | ¹⁹F NMR (376 MHz, DMSO- | |
| | aminobicyclo[1.1.1]pentan-1- | d₆) δ -56.9 | |
| | yl)-6-(3-methyl-5- | | |
| | (trifluoromethoxy)-1H-indol-2- | | |
| | yl)pyrazine-2-carboxamide | | |
| 65 | 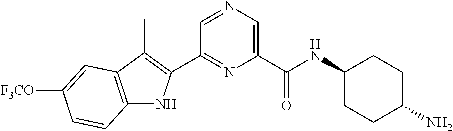 [SMILES: Cc1c(-c2cncc(C(=O)N[C@H]3CC[C@H](N)CC3)n2)[nH]c2ccc(OC(F)(F)F)cc12] | ¹H NMR (400 MHz, DMSO- d₆) δ 12.28 (s, 1H), 9.31 (s, 1H), 9.05 (s, 1H), 9.02 (d, J = 8.6 Hz, 1H), 7.97 (d, J = 5.4 Hz, 3H), 7.69 (d, J = 2.2 Hz, 1H), 7.61 (d, J = 8.8 Hz, 1H), 7.20 (dd, J = 8.8, 2.3 Hz, 1H), 3.89-3.80 (m, | MS m/z calcd for C₂₁H₂₂F₃N₅O₂ 433.2 found 434.2 [M + H]⁺ |
| | N-((1r,4r)-4-aminocyclohexyl)- | 2H), 2.66 (s, 3H), 2.09- | |
| | 6-(3-methyl-5- | 1.92 (m, 4H), 1.75-1.63 | |
| | (trifluoromethoxy)-1H-indol-2- | (m, 2H), 1.56-1.43 (m, | |
| | yl)pyrazine-2-carboxamide | 2H). | |
| | | ¹⁹F NMR (376 MHz, DMSO- | |
| | | d₆) δ -56.9 | |
| 66 | 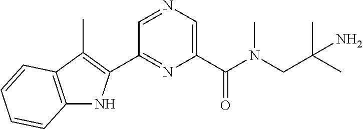 [SMILES: Cc1c(-c2cncc(C(=O)N(C)CC(C)(C)N)n2)[nH]c2ccccc12] | ¹H NMR (400 MHz, DMSO- d₆) δ 11.57 (d, J = 7.7 Hz, 1H), 9.19 (s, 1H), 8.79 (s, 1H), 8.16 (s, 2H), 7.64 (d, J = 7.9 Hz, 1H), 7.49 (d, J = 8.3 Hz, 1H), 7.20 (t, J = 7.6 Hz, 1H), 7.06 (t, J = 7.5 Hz, 1H), 3.72 (s, 2H), 3.20 (s, | MS m/z calcd for C₁₉H₂₃N₅O 337.2 found 338.4 [M + H]⁺ |
| | N-(2-amino-2-methylpropyl)- | 3H), 2.64 (s, 3H), 1.38 (s, | |
| | N-methyl-6-(3-methyl-1H- | 6H). | |
| | indol-2-yl)pyrazine-2- | | |
| | carboxamide | | |
| 67 | 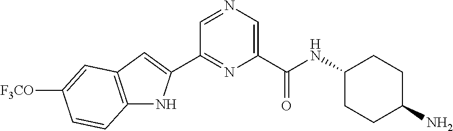 [SMILES: N[C@H]1CC[C@H](NC(=O)c2cncc(-c3cc4cc(OC(F)(F)F)ccc4[nH]3)n2)CC1] | ¹H NMR (400 MHz, DMSO-d₆) δ 12.58 (s, 1H), 9.50 (s, 1H), 9.08 (d, J = 8.4 Hz, 1H), 9.03 (s, 1H), 7.97 (d, J = 5.3 Hz, 3H), 7.65 (d, J = 7.8 Hz, 2H), 7.55 (s, 1H), 7.21 (d, J = 9.2 Hz, 1H), 3.93-3.82 (m, 1H), | MS m/z calcd for C_{2O}H₂₀F₃N₅O₂ 419.2 found 420.1 [M + H]⁺ |
| | N-((1r,4r)-4- | 3.11-2.97 (m, 1H), 2.05 (d, | |
| | aminocyclohexyl)-6-(5- | J = 12.4 Hz, 3H), 1.97 (d, | |
| | (trifluoromethoxy)-1H-indol- | J = 12.9 Hz, 2H), 1.71 (q, J = | |
| | 2-yl)pyrazine-2-carboxamide | 12.4 Hz, 2H), 1.49 (q, J = | |
| | | 12.6 Hz, 2H). | |
| | | ¹⁹F NMR (376 MHz, | |
| | | DMSO-d₆) δ -56.9 | |
| 68 | 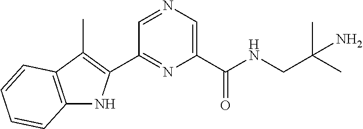 [SMILES: Cc1c(-c2cncc(C(=O)NCC(C)(C)N)n2)[nH]c2ccccc12] | ¹H NMR (400 MHz, DMSO-d₆) δ 11.75 (s, 1H), 9.30 (s, 1H), 9.02 (s, 1H), 8.98 (t, J = 6.7 Hz, 1H), 7.69 (d, J = 8.0 Hz, 1H), 7.50 (d, J = 8.2 Hz, 1H), 7.25 (t, J = 7.5 Hz, 1H), 7.09 (t, J = 7.5 Hz, 1H), | MS m/z calcd for C₁₉H₂₂N₄O 322.2 found 323.1 [M + H]⁺ |
| | 6-(3-methyl-1H-indol-2-yl)- | 3.27 (d, J = 6.7 Hz, 2H), | |
| | N-neopentylpyrazine-2- | 2.68 (s, 3H), 0.96 (s, 9H). | |
| | carboxamide | | |
| 69 | 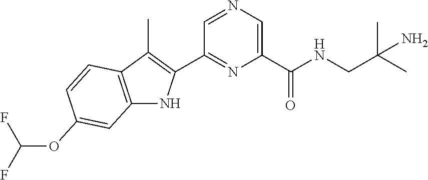 [SMILES: Cc1c(-c2cncc(C(=O)NCC(C)(C)N)n2)[nH]c2cc(OC(F)F)ccc12] | ¹H NMR (400 MHz, DMSO- d₆) δ 12.05 (s, 1H), 9.46 (t, J = 6.7 Hz, 1H), 9.31 (s, 1H), 9.05 (s, 1H), 7.95 (s, 3H), 7.73 (d, J = 8.7 Hz, 1H), 7.25 (s, 1H), 7.22 (t, JHF = 74.0 Hz, 1H), 6.93 (dd, J = 8.8, 2.1 Hz, 1H), 3.57 (d, J = 6.4 Hz, 2H), 2.67 (s, 3H), 1.31 (s, 6H). ¹⁹F NMR (376 MHz, DMSO- | MS m/z calcd for C₁₉H₂₁F₂N₅O₂ 389.2 found 390.1 [M + H]⁺ |
| | N-(2-Amino-2-methylpropyl)- | d₆) δ -80.9, -81.2 | |
| | 6-(6-(difluoromethoxy)-3- | | |
| | methyl-1H-indol-2- | | |
| | yl)pyrazine-2-carboxamide | | |
| 70 | 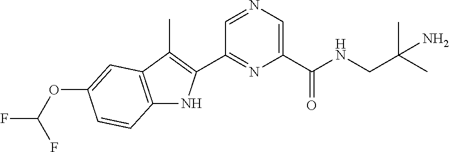 [SMILES: Cc1c(-c2cncc(C(=O)NCC(C)(C)N)n2)[nH]c2ccc(OC(F)F)cc12] | ¹H NMR (400 MHz, DMSO-d₆) δ 12.24 (s, 1H), 9.66 (t, J = 6.8 Hz, 1H), 9.32 (s, 1H), 9.06 (s, 1H), 8.07 (s, 3H), 7.54 (d, J = 8.8 Hz, 1H), 7.47 (s, 1H), 7.16 (t, JHF = 75.0 Hz, 1H), 7.06 (d, J = 8.7 Hz, 1H), 3.59 (d, J = 6.7 Hz, 2H), | LCMS (ESI) m/z calcd for C₁₉H₂₁F₂N₅O₂ 389.2 found 390.2 [M + H]⁺ |
| | N-(2-amino-2-methylpropyl)- | 3.55 (s, 3H), 2.65 (s, 3H), | |
| | 6-(5-(difluoromethoxy)-3- | 1.30 (s, 6H). 19F NMR (376 | |
| | methyl-1H-indol-2- | MHz, DMSO-d₆) δ -80.4, | |
| | yl)pyrazine-2-carboxamide | -80.6 | |

### Example 71: N-(2-amino-2-methylpropyl)-6-(5-(aminomethyl)-3-methyl-1H-indol-2-yl)pyrazine-2-carboxamide

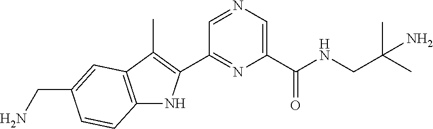 [SMILES: Cc1c(-c2cncc(C(=O)NCC(C)(C)N)n2)[nH]c2ccc(CN)cc12]

The title compound was prepared in the following way:

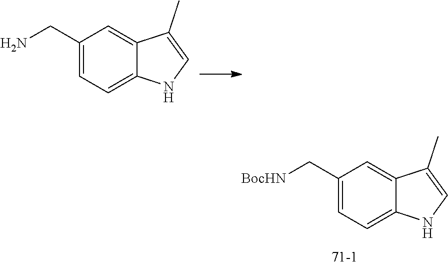 [SMILES: Cc1c[nH]c2ccc(CN)cc12 ; Cc1c[nH]c2ccc(CNC(=O)OC(C)(C)C)cc12]

To a mixture of (3-methyl-1H-indol-5-yl) methanamine (300 mg, 1.872 mmol, CAS 933735-99-0) in CH₃CN (2 mL) was added a solution of Boc₂O (409 mg, 1.872 mmol) in CH₃CN (3 mL) at rt. The reaction mixture was sonicated to crash the starting amine, then stirred at rt for 20 min. The reaction mixture was concentrated in vacuo. The residue was purified by flash chromatography over silica gel (EtOAc/heptane gradient 0 to 50%) to afford the title compound (517 mg, 95% yield). ¹H NMR (500 MHZ, DMSO-d₆) δ 10.64 (s, 1H), 7.32 (s, 1H), 7.31-7.27 (m, 1H), 7.25 (d, J=8.3 Hz, 1H), 7.07 (dd, J=2.4, 1.2 Hz, 1H), 6.98 (dd, J=8.4, 1.6 Hz, 1H), 4.19 (d, J=6.2 Hz, 2H), 2.23 (d, J=1.0 Hz, 3H), 1.40 (s, 9H).

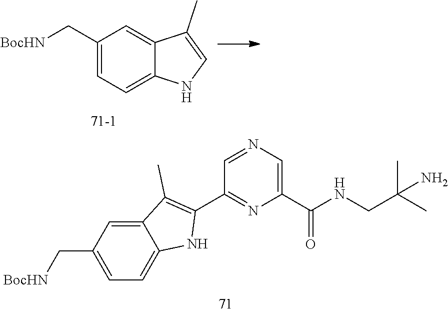 [SMILES: Cc1c[nH]c2ccc(CNC(=O)OC(C)(C)C)cc12 ; Cc1c(-c2cncc(C(=O)NCC(C)(C)N)n2)[nH]c2ccc(CNC(=O)OC(C)(C)C)cc12]

The title compound was prepared from Compound 71-1 in a manner analogous to Example 45. ¹H NMR (500 MHZ, DMSO-d₆) δ 12.35 (s, 1H), 9.78 (t, J=6.6 Hz, 1H), 9.35 (s, 1H), 9.08 (s, 1H), 8.22 (s, 3H), 8.15 (s, 3H), 7.82 (s, 1H), 7.59 (d, J=8.3 Hz, 1H), 7.34 (dd, J=8.5, 1.7 Hz, 1H), 4.13 (q, J=5.7 Hz, 2H), 3.62 (d, J=6.6 Hz, 2H), 2.71 (s, 3H), 1.33 (s, 6H). LCMS (ESI) m/z calcd for C₁₉H₂₄N₆O 352.2 found 353.3 [M+H]⁺.

### Example 72: Ethyl 5-(6-((2-amino-2-methylpropyl)carbamoyl)pyrazin-2-yl)-6-methyl-4H-thieno[3,2-b]pyrrole-2-carboxylate

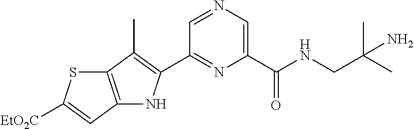 [SMILES: CCOC(=O)c1cc2[nH]c(-c3cncc(C(=O)NCC(C)(C)N)n3)c(C)c2s1]

The title compound was prepared in the following way:

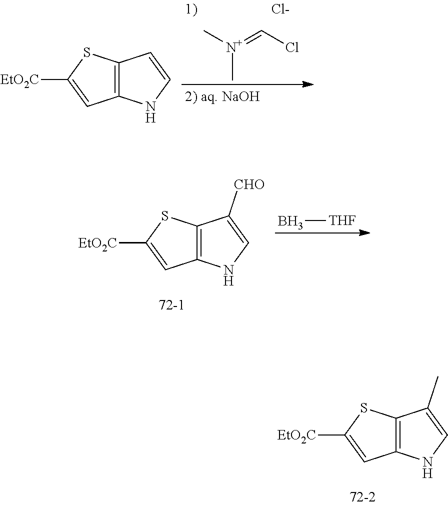

To a solution of ethyl 4H-thieno[3,2-b]pyrrole-2-carboxylate (300 mg, 1.537 mmol) in CH₃CN (1 mL) was added a suspension of N-(chloromethylene)-N-methylmethanaminium chloride (295 mg, 2.305 mmol) in CH₃CN (1 mL). The mixture was stirred at rt for 50 min. Water (1 mL) was added, then the reaction mixture was basified with aq. NaOH (2 M) to pH>6, and was stirred at 100° C. for 20 min. After the bulk of solvent was concentrated. The residue was purified by flash chromatography over silica gel (EtOAc/heptane gradient 0 to 100%) to afford ethyl 6-formyl-4H-thieno[3,2-b]pyrrole-2-carboxylate (227 mg). LCMS (ESI) m/z calcd for C₁₀H₉NO₃S 223.0, found 224.0 (M+H)⁺. To a solution of Compound 72-1 in THF (3 mL) was added BH₃-THF (2 mL, 2 mmol, 1 M) at 0° C. The reaction mixture was stirred at rt for 1 h. The reaction was quenched with aq. NH₄Cl, and the mixture was extracted with EtOAc. The organic layer was washed with water and brine, dried over Na₂SO₄, filtered, and concentrated in vacuo.

The residue was purified by flash chromatography over silica gel (EtOAc/heptane gradient 0 to 30%) to afford ethyl 6-methyl-4H-thieno[3,2-b]pyrrole-2-carboxylate (46 mg, 14% yield in 2 steps). LCMS (ESI) m/z calcd for C₁₀H₁₁NO₂S 209.1, found 210.0 (M+H)⁺.

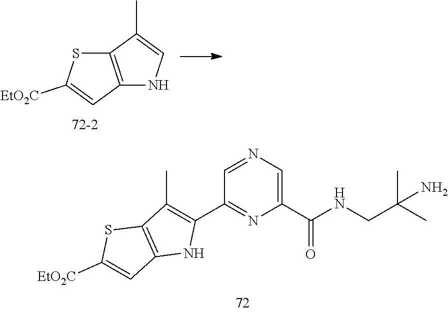 [SMILES: CCOC(=O)c1cc2[nH]cc(C)c2s1 ; CCOC(=O)c1cc2[nH]c(-c3cncc(C(=O)NCC(C)(C)N)n3)c(C)c2s1]

The title compound was prepared from Compound 72-2 in a manner analogous to Example 45. ¹H NMR (500 MHZ, DMSO-d₆) δ 12.58 (s, 1H), 9.55 (t, J=6.6 Hz, 1H), 9.23 (s, 1H), 9.03 (s, 1H), 8.02 (s, 3H), 7.74 (s, 1H), 4.32 (q, J=7.1 Hz, 2H), 3.58 (s, 2H), 2.61 (s, 3H), 1.34 (t, J=7.2 Hz, 3H), 1.32 (s, 6H). LCMS (ESI) m/z calcd for C₁₉H₂₃N₅O₃S 401.2 found 402.1 [M+H]⁺

### Example 73: N-(2-amino-2-methylpropyl)-6-(2,6-dimethyl-4H-thieno[3,2-b]pyrrol-5-yl)pyrazine-2-carboxamide

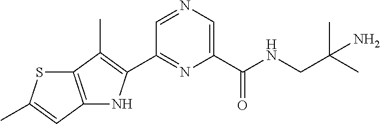 [SMILES: Cc1cc2[nH]c(-c3cncc(C(=O)NCC(C)(C)N)n3)c(C)c2s1]

The title compound was prepared in the following way:

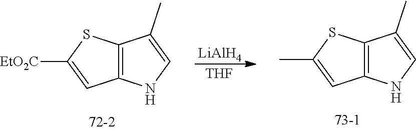

To a solution of Compound 72-2 (308 mg, 1.472 mmol) in THF (5 mL) was added LiAlH₄ (3 mL, 6.90 mmol, 2.3 M in THF) at 0° C. The reaction mixture was stirred at 70° C. for 2.5 h. The reaction was quenched with Na₂SO₄·10H₂O. The mixture was diluted with EtOAc, and filtered through celite pad, washed with EtOAc. The filtrate was concentrated in vacuo. The residue was purified by flash chromatography over silica gel (EtOAc/heptane gradient 0 to 20%) to afford 2,6-dimethyl-4H-thieno[3,2-b]pyrrole (154 mg, 69% yield) as a colorless oil. LCMS (ESI) m/z calcd for C₈H₉NS 151.1, found 152.1 (M+H)⁺.

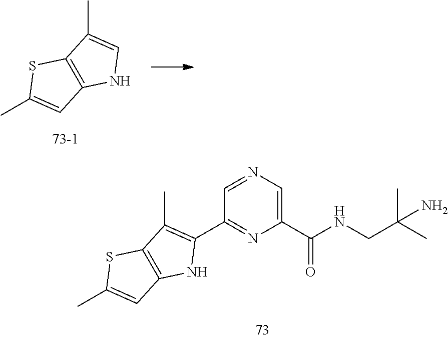

The title compound was prepared from Compound 73-1 in a manner analogous to Example 45. ¹H NMR (500 MHZ, DMSO-d₆) δ 11.94 (s, 1H), 9.49 (t, J=6.7 Hz, 1H), 9.08 (s, 1H), 8.88 (s, 1H), 8.01 (s, 3H), 6.86 (d, J=1.4 Hz, 1H), 3.57 (d, J=6.7 Hz, 2H), 2.54 (s×2, 6H), 1.31 (s, 6H). LCMS (ESI) m/z calcd for C₁₇H₂₁N₅OS 343.2 found 344.3 [M+H]⁺.

### Examples 74: N-(2-Amino-2-methylpropyl)-6-(5-bromo-3-methyl-1H-indol-2-yl)pyrazine-2-carboxamide; and

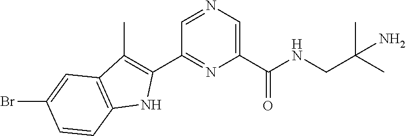 [SMILES: Cc1c(-c2cncc(C(=O)NCC(C)(C)N)n2)[nH]c2ccc(Br)cc12]

### Example 75: N-(2-Amino-2-methylpropyl)-6-(3-methyl-5-(methylsulfonyl)-1H-indol-2-yl)pyrazine-2-carboxamide

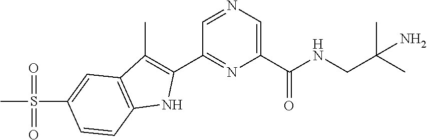 [SMILES: Cc1c(-c2cncc(C(=O)NCC(C)(C)N)n2)[nH]c2ccc(S(C)(=O)=O)cc12]

The title compounds were prepared in the following way:

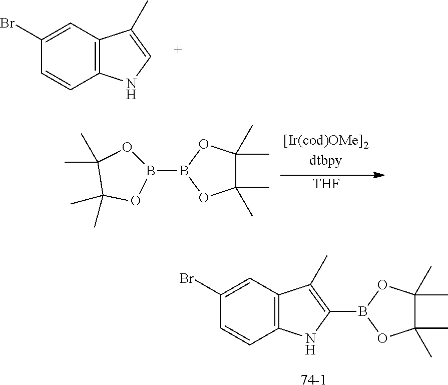

B₂(Pin)₂ (116 mg, 0.457 mmol), [Ir(cod)OMe]₂ (10.1 mg, 0.015 mmol), and dtbpy (8.2 mg, 0.030 mmol) were placed in a 8 mL vial, and the vial was purged by N₂, then THF (2.5 mL) was added. After being stirred at rt for 5 min, 5-(methylsulfonyl)-1H-indole (160 mg, 0.762 mmol) was added. N₂ was bubbled into the reaction mixture. The reaction mixture was stirred at 85° C. for 13 h., and concentrated in vacuo. The residue was purified by flash chromatography over silica gel (EtOAc/heptane gradient 0 to 40%) to afford 5-bromo-3-methyl-2-(4,4,5,5-tetramethyl-1,3,2-dioxaborolan-2-yl)-1H-indole (64 mg, 25% yield) as a colorless oil. LCMS (ESI) m/z calcd for C₁₅H₁₉B⁷⁹BrNO₂ 335.1, found 336.1 (M+H)⁺.

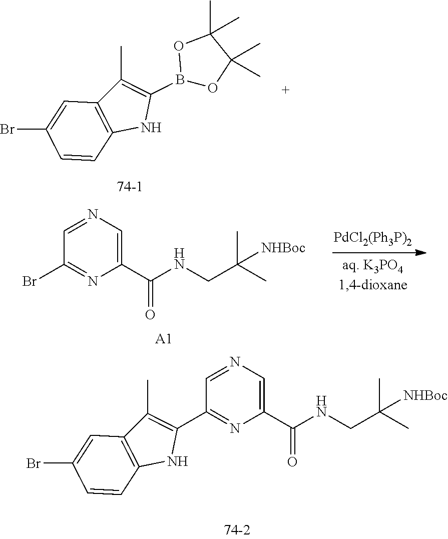

Compound A1 (65 mg, 0.174 mmol), PdCl₂(Ph₃P)₂ (6.1 mg, 0.0087 mmol), and aq. K₃PO₄ (0.17 mL, 0.34 mmol, 2 M) were placed in a 8 mL vial. A solution of Compound 74-1 (64 mg, 0.19 mmol) in 1,4-dioxane (0.7 mL) was added. The vial was capped, and was purged with N₂, then the reaction mixture was stirred at 100° C. for 30 min. The reaction mixture was directly loaded to silica gel and purified by flash chromatography over silica gel (EtOAc/heptane gradient 0 to 50%) to afford tert-butyl (1-(6-(5-bromo-3-methyl-1H-indol-2-yl)pyrazine-2-carboxamido)-2-methylpropan-2-yl) carbamate (63 mg, 72% yield). LCMS (ESI) m/z calcd for C₂₃H₂₈⁸¹BrN₅O₃ 503.1, found 504.3 (M+H)⁺.

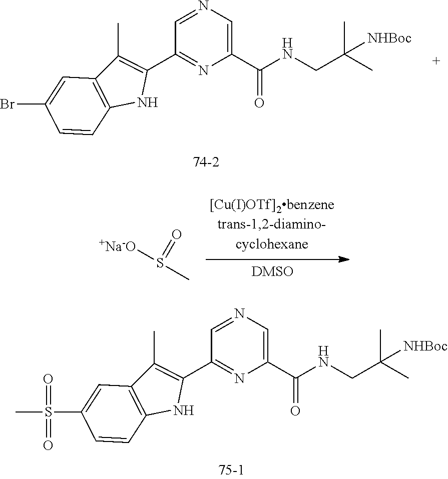

Compound 74-2 (24 mg, 0.048 mmol) and sodium methanesulfinate (9.8 mg, 0.096 mmol) were placed in a 8 mL vial. The vial was capped and purged with N₂, and DMSO (0.3 mL) was added. To this mixture was added trans-1,2-diaminocyclohexane (0.0023 mL, 0.019 mmol) followed by [Cu(I)(OTf)]₂·benzene (2.4 mg, 0.0048 mmol). The reaction mixture was stirred at 100° C. for 14 h. After dilution with EtOAc, the mixture was washed with water (×2) and brine, dried over Na₂SO₄, filtered, and concentrated in vacuo. The residue was purified by flash chromatography over silica gel (EtOAc/heptane gradient 0 to 100%) to afford tert-butyl (2-methyl-1-(6-(3-methyl-5-(methylsulfonyl)-1H-indol-2-yl)pyrazine-2-carboxamido) propan-2-yl) carbamate (3.9 mg, 67% purity, mixture with the unreacted-Br). LCMS (ESI) m/z calcd for C₂₄H₃₁N₅O₅S 501.2, found 502.3 (M+H)⁺.

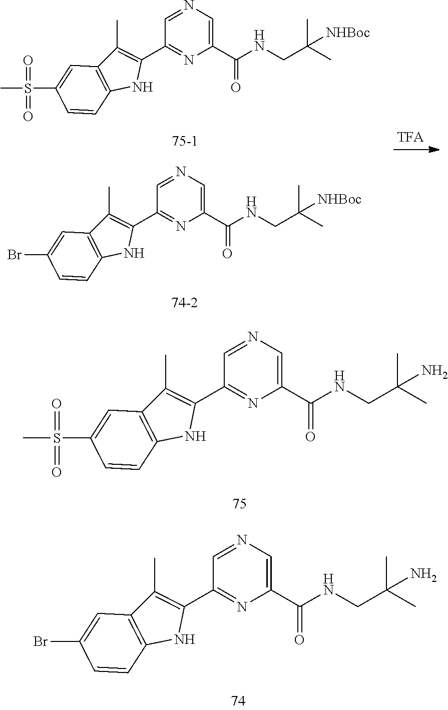 [SMILES: Cc1c(-c2cncc(C(=O)NCC(C)(C)NC(=O)OC(C)(C)C)n2)[nH]c2ccc(S(C)(=O)=O)cc12 ; Cc1c(-c2cncc(C(=O)NCC(C)(C)NC(=O)OC(C)(C)C)n2)[nH]c2ccc(Br)cc12 ; Cc1c(-c2cncc(C(=O)NCC(C)(C)N)n2)[nH]c2ccc(S(C)(=O)=O)cc12 ; Cc1c(-c2cncc(C(=O)NCC(C)(C)N)n2)[nH]c2ccc(Br)cc12]

A mixture of Compound 75-1 and Compound 74-2 (19 mg) in TFA (0.4 mL) was stirred at rt for 30 min. The mixture was concentrated in vacuo and the residue was purified by prep-HPLC to afford the title compounds.

Example 74: ¹H NMR (500 MHZ, DMSO-d₆) δ 11.87 (s, 1H), 9.35 (s, 1H), 9.33 (d, J=6.9 Hz, 1H), 9.10 (s, 1H), 7.92 (d, J=1.9 Hz, 1H), 7.88 (s, 3H), 7.47 (d, J=8.6 Hz, 1H), 7.36 (dd, J=8.6, 1.9 Hz, 1H), 3.59 (d, J=6.6 Hz, 2H), 2.67 (s, 3H), 1.33 (s, 6H). LCMS (ESI) m/z calcd for C₁₈H₂₀⁸¹BrN₅O 403.1 found 404.2 [M+H]⁺.

Example 75: ¹H NMR (500 MHZ, DMSO-d₆) δ 12.22 (s, 1H), 9.41 (s, 1H), 9.35 (t, J=6.7 Hz, 1H), 9.14 (s, 1H), 8.33 (d, J=1.7 Hz, 1H), 7.88 (s, 3H), 7.77 (dd, J=8.6, 1.7 Hz, 1H), 7.72 (d, J=8.6 Hz, 1H), 3.60 (d, J=6.7 Hz, 2H), 3.24 (s, 3H), 2.77 (s, 3H), 1.33 (s, 6H). LCMS (ESI) m/z calcd for C₁₉H₂₃N₅O₃S 401.2 found 402.3 [M+H]⁺.

### Example 76: N-(2-Amino-2-methylpropyl)-6-(3,6,6-trimethyl-4,5,6,7-tetrahydro-1H-indol-2-yl)pyrazine-2-carboxamide

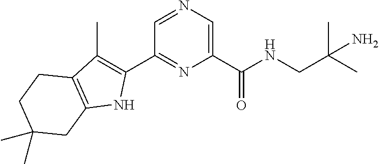 [SMILES: Cc1c(-c2cncc(C(=O)NCC(C)(C)N)n2)[nH]c2c1CCC(C)(C)C2]

The title compound was prepared in the following way:

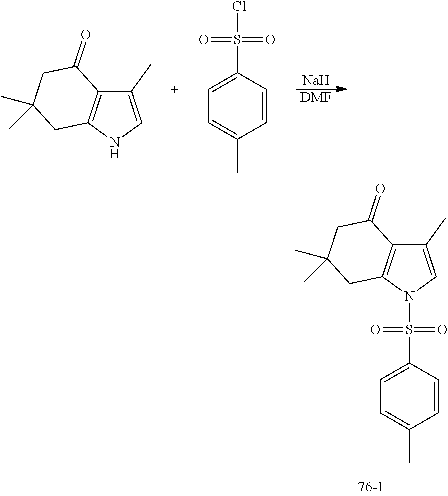

To a solution of 3,6,6-trimethyl-1,5,6,7-tetrahydro-4H-indol-4-one (206 mg, 1.162 mmol) in DMF (3 mL) was added NaH (70 mg, 1.743 mmol, 60% dispersion in mineral oil) at rt. After being stirred at rt for 5 min, 4-methylbenzenesulfonyl chloride (332 mg, 1.743 mmol) was added.

The reaction mixture was stirred at rt for 1.5 h. After dilution with EtOAc, the mixture was washed with aq. KHSO₄, water, sat. aq. NaHCO₃, water, and brine, dried over Na₂SO₄, filtered, and concentrated in vacuo to afford 3,6,6-trimethyl-1-tosyl-1,5,6,7-tetrahydro-4H-indol-4-one (409 mg, quant.). The product was used for the next reaction without purification. LCMS (ESI) m/z calcd for C₁₈H₂₁NO₃S 331.1, found 332.3 (M+H)⁺.

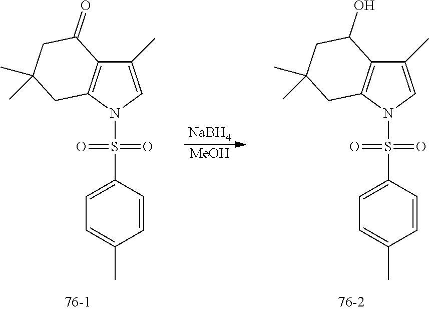

To a suspension of Compound 76-1 (409 mg) in MeOH was added NaBH₄ (80 mg, 2.115 mmol) at 0° C. After being stirred at rt for 40 min, NaBH₄ (40 mg, 1.058 mmol) was added. After 5 min, NaBH₄ (140 mg, 3.701 mmol) and THF (2 mL) were added. After 40 min, NaBH₄ (170 mg, 4.494 mmol) was added. The reaction mixture was stirred at rt for 70 min. The reaction was quenched with aq. KHSO₄, and the reaction mixture was diluted with water, extracted with EtOAc. The organic layer was washed with water and brine, dried over Na₂SO₄, filtered, and concentrated in vacuo. The residue was purified by flash chromatography over silica gel (EtOAc/heptane gradient 0 to 50%) to afford 3,6,6-trimethyl-1-tosyl-4,5,6,7-tetrahydro-1H-indol-4-ol (364 mg, 88% yield in 2 steps). ¹H NMR (500 MHZ, Chloroform-d) δ 7.66-7.61 (m, 2H), 7.30-7.26 (m, 2H), 6.96 (q, J=1.0 Hz, 1H), 2.53-2.49 (m, 2H), 2.41 (s, 3H), 2.10 (d, J=1.2 Hz, 3H), 1.82 (dd, J=13.1, 5.9 Hz, 1H), 1.44 (dd, J=13.2, 7.1 Hz, 1H), 1.03 (s, 3H), 0.86 (s, 3H).

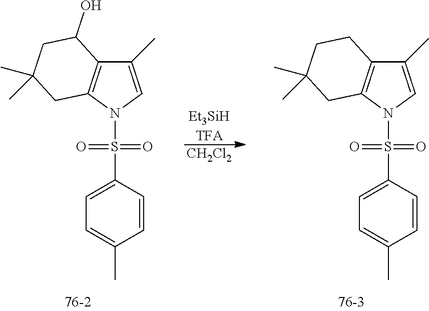

To a solution of Compound 76-2 (128 mg, 0.384 mmol) in CH₂Cl₂ (1 mL) was added Et₃SiH (0.31 mL, 1.92 mmol) followed by TFA (0.15 mL, 1.92 mmol) dropwise. The reaction mixture was stirred at rt for 20 min.

The same reaction was repeated with Compound 76-2 (245 mg, 0.735 mmol), Et₃SiH (0.31 mL, 1.92 mmol), and TFA (0.15 mL, 1.92 mmol) in CH₂Cl₂ (2 mL) at rt for 10 min.

Both reaction mixtures were combined, and concentrated in vacuo. The residue was purified by flash chromatography over silica gel (EtOAc/heptane gradient 0 to 20%) to afford 3,6,6-trimethyl-1-tosyl-4,5,6,7-tetrahydro-1H-indole (217 mg, 63% yield). LCMS (ESI) m/z calcd for C₁₈H₂₃NO₂S 317.1, found 318.2 (M+H)⁺.

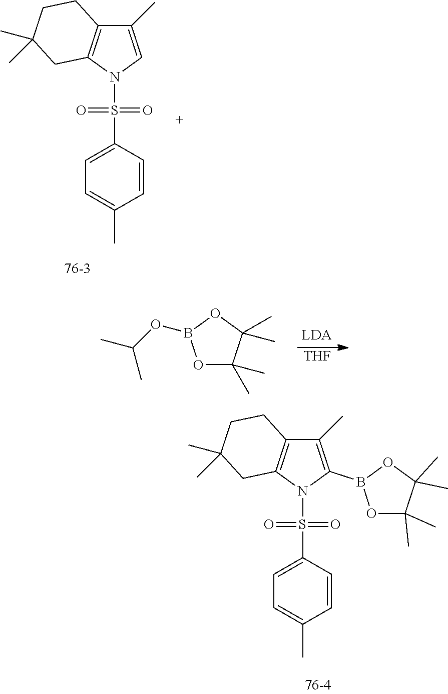

To a solution of Compound 76-3 (217 mg, 0.684 mmol) in THF (2 mL) was added 2-isopropoxy-4,4,5,5-tetramethyl-1,3,2-dioxaborolane (0.28 mL, 1.367 mmol). After cooling to −78° C., LDA (1 mL, 2.0 mmol, 2.0 M in THF/heptane/ethylbenzene) was added to this reaction mixture via syringe. The reaction mixture was stirred at −78° C. to −32° C. for 20 min. The reaction was quenched with aq. KHSO₄, and the resulting mixture was extracted with EtOAc. The organic layer was washed with water and brine. The combined aq. layers were extracted with EtOAc. The combined organic extracts were dried over Na₂SO₄, filtered, and concentrated in vacuo. The residue was purified by flash chromatography over silica gel (EtOAc/heptane gradient 0 to 10%) to afford 3,6,6-trimethyl-2-(4,4,5,5-tetramethyl-1,3,2-dioxaborolan-2-yl)-1-tosyl-4,5,6,7-tetrahydro-1H-indole (71 mg, 23% yield) as a purple oil. LCMS (ESI) m/z calcd for C₂₄H₃₄BNO₄S 443.2, found 444.3 (M+H)⁺.

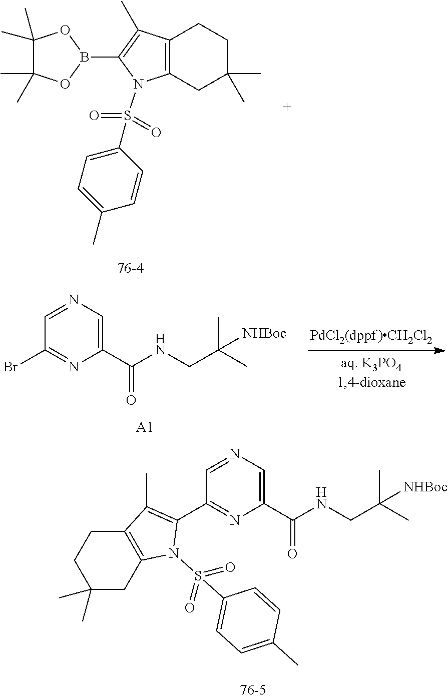

Compound 76-4 (70 mg, 0.158 mmol), Compound A1 (50 mg, 0.134 mmol), and PdCl₂(dppf)·CH₂Cl₂ (5.5 mg, 0.0067 mmol) were placed in a vial. The vial was capped and was purged with N₂. To this mixture was added 1,4-dioxane (0.4 mL) followed by aq. K₃PO₄ (0.32 mL, 0.64 mmol, 2 M). The reaction mixture was stirred at 100° C. for 55 min., and concentrated in vacuo. The residue was purified by flash chromatography over silica gel (EtOAc/heptane gradient 0 to 75%) to afford tert-butyl (2-methyl-1-(6-(3,6,6-trimethyl-1-tosyl-4,5,6,7-tetrahydro-1H-indol-2-yl)pyrazine-2-carboxamido) propan-2-yl) carbamate (74 mg, 91% yield) as a yellow oil. LCMS (ESI) m/z calcd for C₃₂H₄₃N₅O₅S 609.3, found 610.3 (M+H)⁺.

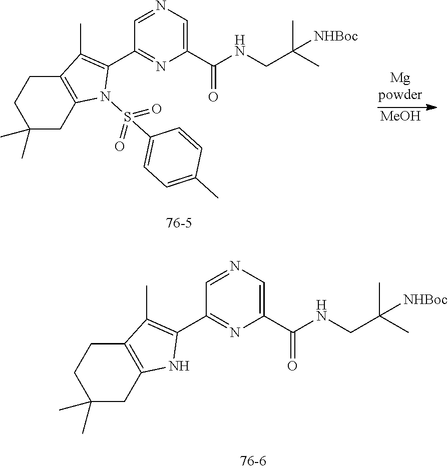 [SMILES: "MeOH" ; Cc1ccc(S(=O)(=O)n2c3c(c(C)c2-c2cncc(C(=O)NCC(C)(C)NC(=O)OC(C)(C)C)n2)CCC(C)(C)C3)cc1 ; Cc1c(-c2cncc(C(=O)NCC(C)(C)NC(=O)OC(C)(C)C)n2)[nH]c2c1CCC(C)(C)C2]

To a solution of Compound 76-5 (70 mg, 0.115 mmol) in MeOH (1 mL) was added Mg powder (28 mg, 1.148 mmol). The reaction mixture was stirred at rt for 30 min (EXP071-2), then at 45° C. for 30 min. The reaction was quenched with aq. KHSO₄, and the reaction mixture was diluted with water, and extracted with EtOAc. The organic layer was washed with water and brine, dried over Na₂SO₄, filtered, and concentrated in vacuo. The residue was purified by flash chromatography over silica gel (EtOAc/heptane gradient 0 to 50%) to afford tert-butyl (2-methyl-1-(6-(3,6,6-trimethyl-4,5,6,7-tetrahydro-1H-indol-2-yl)pyrazine-2-carboxamido) propan-2-yl) carbamate (36 mg, 80% purity, 55% yield) as a yellow oil. LCMS (ESI) m/z calcd for C₂₅H₃₇N₅O₃ 455.3, found 456.3 (M+H)⁺.

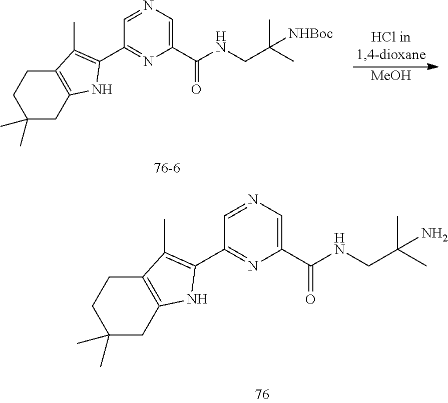 [SMILES: "MeOH" ; Cc1c(-c2cncc(C(=O)NCC(C)(C)NC(=O)OC(C)(C)C)n2)[nH]c2c1CCC(C)(C)C2 ; Cc1c(-c2cncc(C(=O)NCC(C)(C)N)n2)[nH]c2c1CCC(C)(C)C2]

To a solution of Compound 76-6 (36 mg, 0.063 mmol) in MeOH (0.5 mL) was added HCl in 1,4-dioxane (1 mL, 4 mmol, 4 M). The reaction mixture was stirred at rt for 40 min. The reaction mixture was concentrated in vacuo. The residue was triturated with MeOH—CH₃CN, collected by filtration, washed with MeOH—CH₃CN, and dried to give the title compound. ¹H NMR (500 MHZ, DMSO-d₆) δ 11.38 (s, 1H), 9.49 (t, J=6.5 Hz, 1H), 8.91 (s, 1H), 8.75 (s, 1H), 8.06 (s, 3H), 3.55 (d, J=6.7 Hz, 2H), 2.45 (s, 2H), 2.40 (t, J=6.4 Hz, 2H), 2.29 (s, 3H), 1.51 (t, J=6.3 Hz, 2H), 1.29 (s, 6H), 1.00 (s, 6H). LCMS (ESI) m/z calcd for C₂₀H₂₉N₅O 355.2 found 356.3 [M+H]⁺.

### Example 77: N-((1S,2S)-2-Aminocyclopentyl)-6-(6-chloro-5-fluoro-3-methyl-1H-indol-2-yl)pyrazine-2-carboxamide

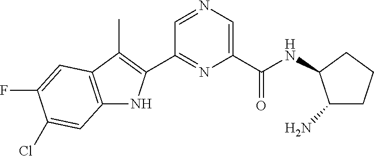 [SMILES: Cc1c(-c2cncc(C(=O)N[C@H]3CCC[C@@H]3N)n2)[nH]c2cc(Cl)c(F)cc12]

The title compound was prepared in the following way:

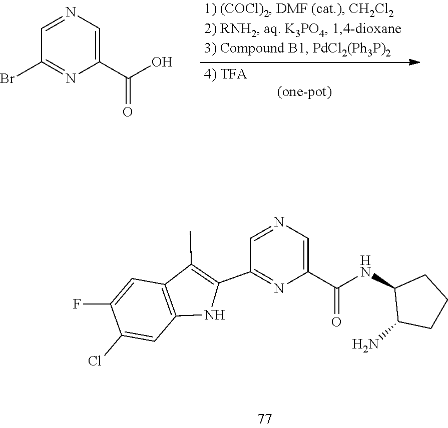 [SMILES: O=C(O)c1cncc(Br)n1 ; Cc1c(-c2cncc(C(=O)N[C@H]3CCC[C@@H]3N)n2)[nH]c2cc(Cl)c(F)cc12]

To a suspension of 6-bromopyrazine-2-carboxylic acid (25 mg, 0.123 mmol) in CH₂Cl₂ (0.31 mL) was added oxalyl chloride (0.043 mL, 0.493 mmol) and one drop of DMF at 0° C. (exothermic reaction). The reaction mixture was stirred at 40° C. for 20 min, and then concentrated in vacuo.

To a solution of tert-butyl ((1S,2S)-2-aminocyclopentyl) carbamate (0.034 mg, 0.172 mmol, CAS 586961-34-4) and aq. K₃PO₄ (0.31 mL, 0.62 mmol, 2M) in 1,4-dioxane (0.31 mL) was added the prepared solution of the —COCl in 1,4-dioxane (0.31 mL) at 0° C. The reaction was warmed up to rt. After being stirred for 30 min, the reaction vessel was flushed with argon before tert-butyl 6-chloro-5-fluoro-3-methyl-2-(4,4,5,5-tetramethyl-1,3,2-dioxaborolan-2-yl)-1H-indole-1-carboxylate (76 mg, 0.185 mmol) and bis(triphenylphosphine) palladium (II) chloride (4.3 mg, 6.16 μmol). The resulting mixture was stirred at 95° C. for 2 h. The organic layer was filtered S-TMT cartridge to remove the residual Pd and then evaporated to dryness. Then the resulting crude material was treated with TFA (0.25 mL) for 2 h until the reaction was completed. The reaction mixture was concentrated in vacuo and the residue was purified by prep-HPLC to afford the title compound (19.7 mg, 31% yield). ¹H NMR (400 MHZ, DMSO-d₆) δ 11.86 (s, 1H), 9.33 (d, J=1.8 Hz, 1H), 9.09 (d, J=1.7 Hz, 1H), 9.06 (d, J=8.1 Hz, 1H), 8.08 (s, 3H), 7.75 (dd, J=10.2, 1.8 Hz, 1H), 7.64 (dd, J=6.4, 1.8 Hz, 1H), 6.52 (s, 2H), 4.38 (q, J=8.2 Hz, 1H), 3.59 (d, J=6.1 Hz, 2H), 2.65 (s, 3H), 2.13 (d, J=9.8 Hz, 2H), 1.90-1.76 (m, 3H), 1.69 (dd, J=13.3, 7.2 Hz, 1H). ¹⁹F NMR (376 MHZ, DMSO-d₆) δ −127.3. LCMS (ESI) m/z calcd for C₁₉H₁₉ClFN₅O 387.1 found 388.3 [M+H]⁺.

Examples 78-82 were prepared from the appropriate amine in a manner analogous to Example 77.

| Example | Structure | NMR | LC-MS |
|---|---|---|---|
| 78 | 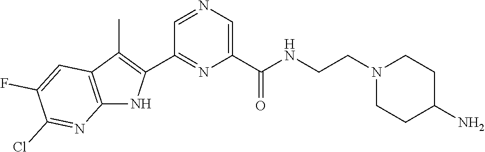 [SMILES: Cc1c(-c2cncc(C(=O)NCCN3CCC(N)CC3)n2)[nH]c2nc(Cl)c(F)cc12] | ¹H NMR (500 MHz, DMSO- d₆) δ 11.90 (s, 1H), 9.62 (s, 1H), 9.48 (t, J = 6.2 Hz, 1H), 9.36 (s, 1H), 9.08 (s, 1H), 8.12 (s, 3H), 7.72 (d, J = 2.3 Hz, 1H), 7.58 (d, J = 8.8 Hz, 1H), 7.26-7.21 (m, 1H), 3.79 (m, 4H), 3.34 (m, 3H), 3.12 (m, 2H), 2.69 (s, 3H), 2.14 (m, | MS m/z calcd for C₂₁H₂₄ClFN₆O 430.2 found 431.4 [M + H]⁺ |
| | N-(2-(4-aminopiperidin-1- | 2H), 1.77 (m, 2H). | |
| | yl)ethyl)-6-(6-chloro-5-fluoro- | ¹⁹F NMR (376 MHz, DMSO- | |
| | 3-methyl-1H-indol-2- | d₆) δ -73.8 (TFA), -127.4 | |
| | yl)pyrazine-2-carboxamide | | |
| 79 | 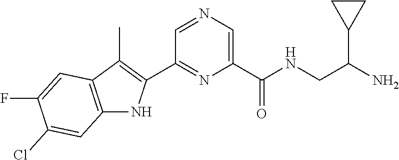 [SMILES: Cc1c(-c2cncc(C(=O)NCC(N)C3CC3)n2)[nH]c2cc(Cl)c(F)cc12] | ¹H NMR (500 MHz, DMSO- d₆) δ 11.86 (s, 1H), 9.36 (s, 1H), 9.33 (t, J = 6.4 Hz, 2H), 9.10 (s, 1H), 7.97 (s, 3H), 7.72 (d, J = 1.9 Hz, 1H), 7.58 (d, J = 8.8 Hz, 1H), 7.23 (d, J = 9.3 Hz, 1H), 3.83-3.65 (m, 2H), 2.69 (s, 3H), 2.68-2.63 (m, 1H), 1.07-0.95 (m, 1H), | MS m/z calcd for C₁₉H₁₉ClFN₅O 387.1 found 388.3 [M + H]⁺ |
| | N-(2-amino-2- | 0.70-0.55 (m, 2H), 0.51- | |
| | cyclopropylethyl)-6-(6- | 0.46 (m, 1H), 0.44-0.34 (m, | |
| | chloro-5-fluoro-3-methyl-1H- | 1H). | |
| | indol-2-yl)pyrazine-2- | ¹⁹F NMR (376 MHz, DMSO- | |
| | carboxamide | d₆) δ -73.5 (TFA), -127.4 | |
| 80 | 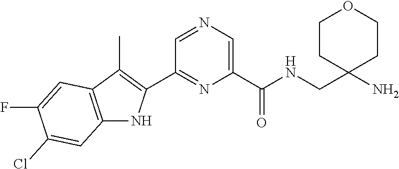 [SMILES: Cc1c(-c2cncc(C(=O)NCC3(N)CCOCC3)n2)[nH]c2cc(Cl)c(F)cc12] | ¹H NMR (400 MHz, DMSO- d₆) δ 11.84 (s, 1H), 9.34 (m, 2H), 9.11 (d, J = 1.7 Hz, 1H), 8.06 (s, 3H), 7.75 (dd, J = 10.1, 1.7 Hz, 1H), 7.62 (dd, J = 6.3, 1.7 Hz, 1H), 3.82- 3.75 (m, 6H), 3.74-3.66 (m, 4H), 2.65 (s, 3H), 1.89-1.81 (m, 2H), 1.79-1.70 (m, 2H). ¹⁹F NMR (376 MHz, DMSO- | MS m/z calcd for C₂₀H₂₁ClFN₅O₂ 417.1 found 418.4 [M + H]⁺ |
| | N-((4-aminotetrahydro-2H- | d₆) δ -73.7 (TFA), -127.3 | |
| | pyran-4-yl)methyl)-6-(6- | | |
| | chloro-5-fluoro-3-methyl-1H- | | |
| | indol-2-yl)pyrazine-2- | | |
| | carboxamide | | |
| 81 | 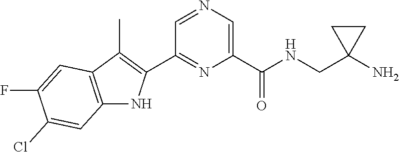 [SMILES: Cc1c(-c2cncc(C(=O)NCC3(N)CC3)n2)[nH]c2cc(Cl)c(F)cc12] | ¹H NMR (500 MHz, DMSO- d₆) δ 11.88 (s, 1H), 9.36 (s, 1H), 9.34 (t, J = 6.5 Hz, 1H), 9.11 (s, 1H), 8.23 (s, 3H), 7.72 (s, 1H), 7.59 (d, J = 8.8 Hz, 1H), 7.23 (ddd, J = 8.8, 2.4, 1.0 Hz, 1H), 3.70 (d, J = 6.4 Hz, 2H), 2.70 (s, 3H), 1.01-0.97 (m, 2H), 0.95- | MS m/z calcd for C₁₈H₁₇ClFN₅O 373.1 found 374.3 [M + H]⁺ |
| | N-((1- | 0.91 (m, 2H). | |
| | aminocyclopropyl)methyl)-6- | ¹⁹F NMR (376 MHz, DMSO- | |
| | (6-chloro-5-fluoro-3-methyl- | d₆) δ -73.5 (TFA), -127.4 | |
| | 1H-indol-2-yl)pyrazine-2- | | |
| | carboxamide | | |
| 82 | 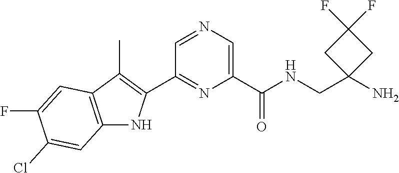 [SMILES: Cc1c(-c2cncc(C(=O)NCC3(N)CC(F)(F)C3)n2)[nH]c2cc(Cl)c(F)cc12] | ¹H NMR (500 MHz, DMSO- d₆) δ 11.82 (s, 1H), 9.53 (t, J = 6.5 Hz, 1H), 9.38 (s, 1H), 9.11 (s, 1H), 8.46 (s, 3H), 7.73 (d, J = 2.3 Hz, 1H), 7.60 (d, J = 8.8 Hz, 1H), 7.26- 7.22 (m, 1H), 3.84 (d, J = 6.5 Hz, 2H), 3.17-3.10 (m, 2H), 2.99-2.92 (m, 2H), 2.70 (s, 3H). | MS m/z calcd for C₁₉H₁₇ClF₃N₅O 423.1 found 424.4 [M + H]⁺ |
| | N-((1-amino-3,3- | | |
| | difluorocyclobutyl)methyl)-6- | | |
| | (6-chloro-5-fluoro-3-methyl- | | |
| | 1H-indol-2-yl)pyrazine-2- | | |
| | carboxamide | | |

### Example 83: N-(2-amino-2-methylpropyl)-6-(3-methyl-1H-indol-2-yl)pyrazine-2-carboxamide

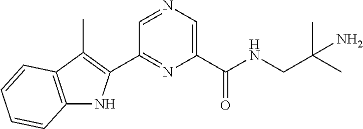 [SMILES: Cc1c(-c2cncc(C(=O)NCC(C)(C)N)n2)[nH]c2ccccc12]

The title compound was prepared in the following way:

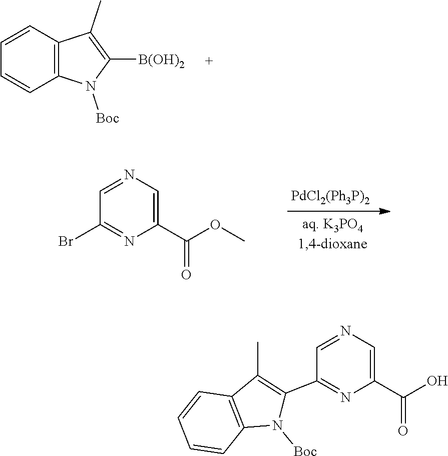 [SMILES: Cc1c(B(O)O)n(C(=O)OC(C)(C)C)c2ccccc12 ; COC(=O)c1cncc(Br)n1 ; Cc1c(-c2cncc(C(=O)O)n2)n(C(=O)OC(C)(C)C)c2ccccc12]

A mixture of (1-(tert-butoxycarbonyl)-3-methyl-1H-indol-2-yl) boronic acid (462 mg, 1.681 mmol, CAS 352359-20-7), methyl 6-bromopyrazine-2-carboxylate (304 mg, 1.401 mmol), and PdCl₂(Ph₃P)₂ (49 mg, 0.070 mmol) in 1,4-dioxane (2 mL)-aq. K₃PO₄ (2.1 mL, 4.2 mmol, 2 M) was stirred at 120° C. for 90 min in a microwave vial. Aq. NaOH (1 mL, 2 mmol, 2 M) was added, and the reaction mixture was stirred at 60° C. for 12 h. The reaction mixture was transferred to a separate funnel with water and Et₂O, and the aq. layer was separated. The aq. layer was acidified with aq. KHSO₄, and extracted with EtOAc (×3). The combined organic extracts were washed with aq. KHSO₄ and brine, dried over Na₂SO₄, filtered, and concentrated in vacuo. The residue was triturated with CH₃CN, and the precipitated product was filtered out. The filtrate was concentrated in vacuo to afford 6-(1-(tert-butoxycarbonyl)-3-methyl-1H-indol-2-yl)pyrazine-2-carboxylic acid (315 mg, 64% yield). LCMS (ESI) m/z calcd for C₁₉H₁₉N₃O₄ 353.1, found 354.0 (M+H)⁺.

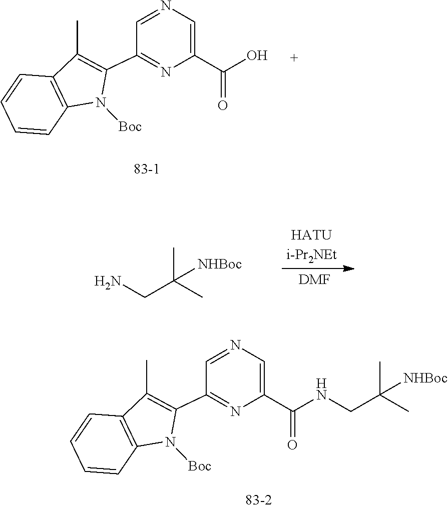

To a mixture of Compound 83-1 (69 mg, 0.195 mmol) and tert-butyl (1-amino-2-methylpropan-2-yl) carbamate (37 mg, 0.195 mmol) in DMF (0.6 mL) at rt was added i-Pr₂NEt (0.068 mL, 0.391 mmol) followed by HATU (89 mg, 0.234 mmol). The reaction mixture was stirred at rt for 3.5 h. After dilution with EtOAc, the mixture was washed with aq. KHSO₄, water, sat. aq. NaHCO₃, water, and brine, dried over Na₂SO₄, filtered, and concentrated in vacuo. The residue was purified by flash chromatography over silica gel (EtOAc/heptane gradient 0 to 50%) to afford tert-butyl 2-(6-((2-((tert-butoxycarbonyl)amino)-2-methylpropyl)carbamoyl)pyrazin-2-yl)-3-methyl-1H-indole-1-carboxylate (62 mg, 60% yield). LCMS (ESI) m/z calcd for C₂₈H₃₇N₅O₅ 523.3, found 524.3 (M+H)⁺.

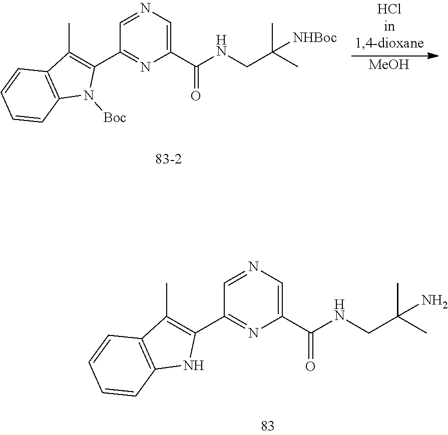 [SMILES: "MeOH" ; Cc1c(-c2cncc(C(=O)NCC(C)(C)NC(=O)OC(C)(C)C)n2)n(C(=O)OC(C)(C)C)c2ccccc12 ; Cc1c(-c2cncc(C(=O)NCC(C)(C)N)n2)[nH]c2ccccc12]

To a solution of Compound 83-2 (61 mg, 0.116 mmol) in MeOH (0.2 mL) was added HCl in 1,4-dioxane (1 mL, 4 mmol, 4 M). The reaction mixture was stirred at rt for 16 h. The reaction mixture was concentrated in vacuo. The residue was triturated with MeOH—CH₃CN, collected by filtration, washed with MeOH—CH₃CN, and dried to afford the title compound (31 mg, 73% yield). ¹H NMR (500 MHZ, DMSO-d₆) δ 12.05 (s, 1H), 9.66 (t, J=6.6 Hz, 1H), 9.33 (s, 1H), 9.05 (s, 1H), 8.09 (s, 3H), 7.69 (d, J=8.0 Hz, 1H), 7.57-7.51 (m, 1H), 7.24 (ddd, J=8.1, 6.9, 1.1 Hz, 1H), 7.08 (ddd, J=8.0, 6.9, 1.0 Hz, 1H), 3.61 (d, J=6.7 Hz, 2H), 2.70 (s, 3H), 1.33 (s, 6H). LCMS (ESI) m/z calcd for C₁₈H₂₁N₅O 323.2 found 324.0 [M+H]⁺.

Examples 84-101 were prepared from the appropriate amine in a manner analogous to Example 83.

| Example | Structure | NMR | LC-MS |
|---|---|---|---|
| 84 | 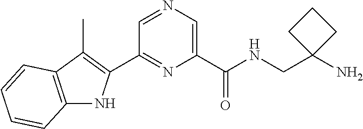 [SMILES: Cc1c(-c2cncc(C(=O)NCC3(N)CCC3)n2)[nH]c2ccccc12] | ¹H NMR (400 MHz, DMSO-d₆) δ 11.64 (s, 1H), 9.50 (t, J = 6.6 Hz, 1H), 9.35 (s, 1H), 9.07 (s, 1H), 8.14 (s, 3H), 7.72 (d, J = 8.0 Hz, 1H), 7.52 (dd, J = 8.2, 1.1 Hz, 1H), 7.27 (ddd, J = 8.1, 6.9, 1.1 Hz, | MS m/z calcd for C₁₉H₂₁N₅O 335.2 found 336.3 [M + H]⁺ |
| | N-((1- | 1H), 7.11 (ddd, J = 8.0, | |
| | aminocyclobutyl)methyl)-6- | 6.9, 1.0 Hz, 1H), 3.80 (d, | |
| | (3-methyl-1H-indol-2- | J = 6.6 Hz, 2H), 2.71 (s, | |
| | yl)pyrazine-2-carboxamide | 3H), 2.34-2.27 (m, 2H), | |
| | | 2.25-2.18 (m, 2H), 1.97- | |
| | | 1.87 (m, 2H). | |
| 85 | 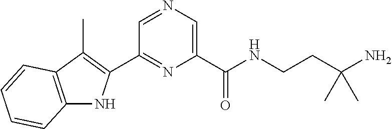 [SMILES: Cc1c(-c2cncc(C(=O)NCCC(C)(C)N)n2)[nH]c2ccccc12] | ¹H NMR (400 MHz, DMSO-d₆) δ 11.98 (s, 1H), 9.65 (t, J = 6.3 Hz, 1H), 9.30 (s, 1H), 9.01 (s, 1H), 8.15 (s, 3H), 7.68 (d, J = 8.2 Hz, 1H), 7.53 (d, J = 8.2 Hz, 1H), 7.24 (t, J = 7.6 Hz, 1H), 7.07 (t, J = | MS m/z calcd for C₁₉H₂₃N₅O 337.2 found 338.2 [M + H]⁺ |
| | N-(3-amino-3-methylbutyl)- | 7.7 Hz, 1H), 3.52 (q, J = | |
| | 6-(3-methyl-1H-indol-2- | 7.2 Hz, 2H), 2.68 (s, 3H), | |
| | yl)pyrazine-2-carboxamide | 1.94 (q, J = 7.2 Hz, 2H), | |
| | | 1.34 (s, 6H). | |
| 86 | 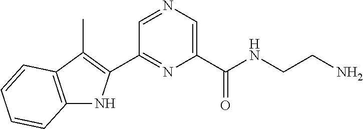 [SMILES: Cc1c(-c2cncc(C(=O)NCCN)n2)[nH]c2ccccc12] | ¹H NMR (500 MHz, DMSO-d₆) δ 12.03 (s, 1H), 9.70 (d, J = 6.6 Hz, 1H), 9.32 (s, 1H), 9.03 (s, 1H), 8.06 (s, 3H), 7.69 (d, J = 8.0 Hz, 1H), 7.54 (d, J = 8.1 Hz, 1H), 7.24 (t, J = 7.6 Hz, 1H), 7.08 (t, J = | MS m/z calcd for C₁₆H₁₇N₅O 295.1 found 296.0[M + H]⁺ |
| | N-(2-aminoethyl)-6-(3- | 7.5 Hz, 1H), 3.70 (d, J = | |
| | methyl-1H-indol-2- | 6.3 Hz, 2H), 3.12 (d, J = | |
| | yl)pyrazine-2-carboxamide | 5.5 Hz, 2H), 2.69 (s, 3H). | |
| 87 | 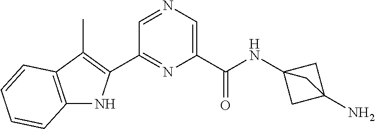 [SMILES: Cc1c(-c2cncc(C(=O)NC34CC(N)(C3)C4)n2)[nH]c2ccccc12] | ¹H NMR (500 MHz, DMSO-d₆) δ 11.79 (s, 1H), 9.82 (s, 1H), 9.32 (d, J = 1.6 Hz, 1H), 9.00 (d, J = 1.6 Hz, 1H), 8.85 (s, 3H), 7.70 (d, J = 8.1 Hz, 1H), 7.53 (d, J = 8.2 Hz, 1H), 7.25 (t, J = 7.6 Hz, | MS m/z calcd for C₁₉H₁₉N₅O 333.2 found 334.1 [M + H]⁺ |
| | N-(3- | 1H), 7.09 (t, J = 7.5 Hz, | |
| | aminobicyclo[1.1.1]pentan- | 1H), 2.68 (s, 3H), 2.46 (d, | |
| | 1-yl)-6-(3-methyl-1H-indol- | J = 1.6 Hz, 6H). | |
| | 2-yl)pyrazine-2- | | |
| | carboxamide | | |
| 88 | 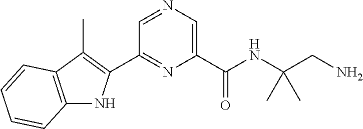 [SMILES: Cc1c(-c2cncc(C(=O)NC(C)(C)CN)n2)[nH]c2ccccc12] | ¹H NMR (500 MHz, DMSO-d₆) δ 11.91 (s, 1H), 9.33 (s, 1H), 9.04 (s, 1H), 8.39 (s, 1H), 8.02 (s, 3H), 7.70 (d, J = 8.0 Hz, 1H), 7.52 (d, J = 8.2 Hz, 1H), 7.25 (t, J = 7.6 Hz, 1H), 7.09 (t, J = 7.5 Hz, | MS m/z calcd for C₁₈H₂₁N₅O 323.2 found 324.0 [M + H]⁺ |
| | N-(1-amino-2- | 1H), 3.37 (d, J = 6.1 Hz, | |
| | methylpropan-2-yl)-6-(3- | 2H), 2.68 (s, 3H), 1.54 (s, | |
| | methyl-1H-indol-2- | 6H). | |
| | yl)pyrazine-2-carboxamide | | |
| 89 | 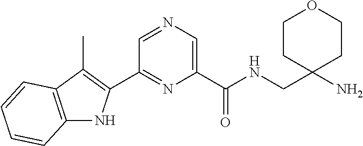 [SMILES: Cc1c(-c2cncc(C(=O)NCC3(N)CCOCC3)n2)[nH]c2ccccc12] | ¹H NMR (500 MHz, DMSO-d₆) δ 11.59 (s, 1H), 9.39 (t, J = 6.6 Hz, 1H), 9.34 (s, 1H), 9.07 (s, 1H), 8.07 (s, 3H), 7.71 (d, J = 8.0 Hz, 1H), 7.49 (d, J = 8.2 Hz, 1H), 7.26 (t, J = 7.6 Hz, 1H), 7.14-7.07 (m, 1H), 3.82-3.77 (m, | MS m/z calcd for C₂₀H₂₃N₅O₂ 365.2 found 366.3 [M + H]⁺ |
| | N-((4-aminotetrahydro-2H- | 4H), 3.71 (ddd, J = 12.0, | |
| | pyran-4-yl)methyl)-6-(3- | 8.3, 3.4 Hz, 2H), 2.70 (s, | |
| | methyl-1H-indol-2- | 3H), 1.86 (dt, J = 14.0, 4.3 | |
| | yl)pyrazine-2-carboxamide | Hz, 2H), 1.79-1.72 (m, | |
| | | 2H). | |
| 90 | 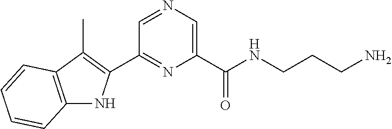 [SMILES: Cc1c(-c2cncc(C(=O)NCCCN)n2)[nH]c2ccccc12] | ¹H NMR (500 MHz, DMSO-d₆) δ 11.86 (s, 1H), 9.59 (t, J = 6.6 Hz, 1H), 9.32 (s, 1H), 9.02 (s, 1H), 7.86 (s, 3H), 7.70 (d, J = 8.0 Hz, 1H), 7.53 (d, J = 8.2 Hz, 1H), 7.25 (t, J = 7.5 Hz, 1H), 7.09 (t, J = | MS m/z calcd for C₁₇H₁₉N₅O 309.2 found 310.0 [M + H]⁺ |
| | N-(3-aminopropyl)-6-(3- | 7.5 Hz, 1H), 3.59-3.44 | |
| | methyl-1H-indol-2- | (m, 2H), 2.94-2.89 (m, | |
| | yl)pyrazine-2-carboxamide | 2H), 2.69 (s, 3H), 2.01- | |
| | | 1.85 (m, 2H). | |
| 91 | 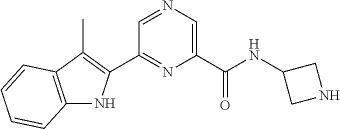 [SMILES: Cc1c(-c2cncc(C(=O)NC3CNC3)n2)[nH]c2ccccc12] | ¹H NMR (400 MHz, DMSO-d₆) δ 11.54 (s, 1H), 9.60 (d, J = 6.6 Hz, 1H), 9.34 (s, 1H), 9.04 (s, 1H), 8.82 (s, 2H), 7.71 (d, J = 8.0 Hz, 1H), 7.50 (d, J = 8.2 Hz, 1H), 7.27 (ddd, J = 8.2, 6.9, 1.2 Hz, 1H), | MS m/z calcd for C₁₇H₁₇N₅O 307.1 found 308.2 [M + H]⁺ |
| | N-(azetidin-3-yl)-6-(3- | 7.11 (ddd, J= 8.0, 7.0, 1.0 | |
| | methyl-1H-indol-2- | Hz, 1H), 4.92-4.82 (m, | |
| | yl)pyrazine-2-carboxamide | 1H), 4.36-4.26 (m, 4H), | |
| | | 2.69 (s, 3H). | |
| 92 | 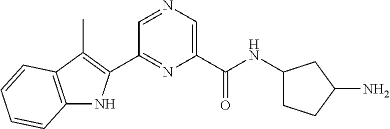 [SMILES: Cc1c(-c2cncc(C(=O)NC3CCC(N)C3)n2)[nH]c2ccccc12] | ¹H NMR (500 MHz, DMSO-d₆) δ 11.69 (s, 1H), 9.31 (s, 1H), 9.03 (d, J = 6.2 Hz, 1H), 9.03 (s, 1H), 8.01 (s, 3H), 7.71 (d, J = 8.0 Hz, 1H), 7.49 (d, J = 8.1 Hz, 1H), 7.27 (t, J = 7.5 Hz, 1H), 7.11 (t, J = | MS m/z calcd for C₁₉H₂₁N₅O 335.2 found 336.3[M + H]⁺ |
| | N-(3-aminocyclopentyl)-6- | 7.5 Hz, 1H), 4.37-4.29 | |
| | (3-methyl-1H-indol-2- | (m, 1H), 3.65-3.57 (m, | |
| | yl)pyrazine-2-carboxamide | 1H), 2.69 (s, 3H), 2.51- | |
| | | 2.44 (m, 1H), 2.14-1.90 | |
| | | (m, 3H), 1.85-1.73 (m, | |
| | | 2H). | |
| 93 | 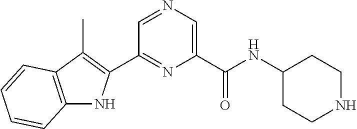 [SMILES: Cc1c(-c2cncc(C(=O)NC3CCNCC3)n2)[nH]c2ccccc12] | ¹H NMR (500 MHz, DMSO-d₆) δ 11.88 (s, 1H), 9.31 (s, 1H), 9.11 (d, J = 8.0 Hz, 1H), 9.02 (s, 1H), 8.77 (br, 1H), 8.61 (br, 1H), 7.70 (d, J = 8.0 Hz, 1H), 7.52 (dd, J = 8.2, 1.0 Hz, 1H), 7.25 (ddd, , | MS m/z calcd for C₁₉H₂₁N₅O 335.2 found 336.0 [M + H]⁺ |
| | 6-(3-methyl-1H-indol-2-yl)- | J = 8.1, 6.9, 1.1 Hz, 1H) | |
| | N-(piperidin-4-yl)pyrazine-2- | 7.09 (ddd, J= 8.1, 6.9, 1.0 | |
| | carboxamide | Hz, 1H), 4.23-4.12 (m, | |
| | | 1H), 3.42-3.37 (m, 2H), | |
| | | 3.14-3.04 (m, 2H), 2.68 | |
| | | (s, 3H), 2.10-2.05 (m, | |
| | | 2H), 2.03-1.91 (m, 2H). | |
| 94 | 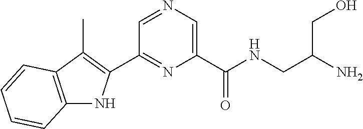 [SMILES: Cc1c(-c2cncc(C(=O)NCC(N)CO)n2)[nH]c2ccccc12] | ¹H NMR (500 MHz, DMSO-d₆) δ 11.59 (s, 1H), 9.35 (t, J = 6.2 Hz, 1H), 9.32 (s, 1H), 9.04 (s, 1H), 7.99 (s, 3H), 7.70 (d, J = 8.0 Hz, 1H), 7.50 (d, J = 8.2 Hz, 1H), 7.26 (t, J = 7.6 Hz, 1H), 7.10 (t, J = | MS m/z calcd for C₁₇H₁₉N₅O₂ 325.2 found 326.3 [M + H]⁺ |
| | N-(2-amino-3- | 7.5 Hz, 1H), 3.75-3.60 | |
| | hydroxypropyl)-6-(3-methyl- | (m, 6H), 2.69 (s, 3H). | |
| | 1H-indol-2-yl)pyrazine-2- | | |
| | carboxamide | | |
| 95 | 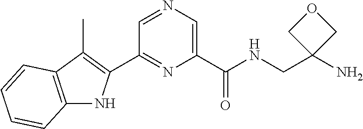 [SMILES: Cc1c(-c2cncc(C(=O)NCC3(N)COC3)n2)[nH]c2ccccc12] | ¹H NMR (500 MHz, DMSO-d₆) δ 11.60 (s, 1H), 9.62 (t, J = 6.5 Hz, 1H), 9.36 (s, 1H), 9.07 (s, 1H), 8.62 (s, 3H), 7.72 (d, J = 8.0 Hz, 1H), 7.52 (d, J = 8.2 Hz, 1H), 7.28 (ddd, J = 8.2, 6.9, 1.1 Hz, 1H), | MS m/z calcd for C₁₈H₁₉N₅O₂ 337.2 found 338.3 [M + H]⁺ |
| | N-((3-aminooxetan-3- | 7.11 (t, J = 7.5 Hz, 1H), | |
| | yl)methyl)-6-(3-methyl-1H- | 4.71 (d, J = 7.4 Hz, 2H), | |
| | indol-2-yl)pyrazine-2- | 4.64 (d, J = 7.4 Hz, 2H), | |
| | carboxamide | 3.97 (d, J = 6.6 Hz, 3H), | |
| | | 2.71 (s, 3H). | |
| 96 | 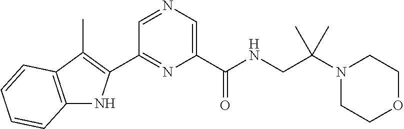 [SMILES: Cc1c(-c2cncc(C(=O)NCC(C)(C)N3CCOCC3)n2)[nH]c2ccccc12] | ¹H NMR (500 MHz, DMSO-d₆) δ 11.69 (s, 1H), 9.35 (s, 1H), 9.35- 9.25 (m, 2H), 9.07 (s, 1H), 7.71 (d, J = 8.0 Hz, 1H), 7.50 (d, J = 8.2 Hz, 1H), 7.26 (t, J = 7.5 Hz, 1H), 7.10 (t, J = 7.5 Hz, 1H), | MS m/z calcd for C₂₂H₂₇N₅O₂ 393.2 found 394.3 [M + H]⁺ |
| | 6-(3-Methyl-1H-indol-2-yl)- | 4.13-4.00 (m, 2H), 3.83- | |
| | N-(2-methyl-2- | 3.72 (m, 4H), 3.67 (d, | |
| | morpholinopropyl)pyrazine- | J = 12.1 Hz, 2H), 3.31- | |
| | 2-carboxamide | 3.21 (m, 2H), 2.70 (s, 3H), | |
| | | 1.43 (s, 6H). | |
| 97 | 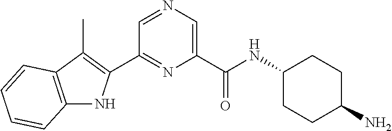 [SMILES: Cc1c(-c2cncc(C(=O)N[C@H]3CC[C@H](N)CC3)n2)[nH]c2ccccc12] | ¹H NMR (500 MHz, DMSO-d₆) δ 11.88 (s, 1H), 9.29 (s, 1H), 9.01 (s, 1H), 8.95 (d, J = 8.1 Hz, 1H), 7.98 (d, J = 4.7 Hz, 3H), 7.69 (d, J = 8.0 Hz, 1H), 7.53 (d, J = 8.1 Hz, 1H), 7.28-7.22 (m, 1H), | MS m/z calcd for C₂₀H₂₃N₅O 349.2 found 350.1 [M + H]⁺ |
| | N-((1r,4r)-4- | 7.09 (t, J = 7.5 Hz, 1H), | |
| | aminocyclohexyl)-6-(3- | 3.92-3.80 (m, 1H), 3.10- | |
| | methyl-1H-indol-2- | 3.01 (m, 1H), 2.67 (s, | |
| | yl)pyrazine-2-carboxamide | 3H), 2.08-2.03 (m, 2H), | |
| | | 2.02-1.96 (m, 2H), 1.72- | |
| | | 1.63 (m, 2H), 1.56- | |
| | | 1.45 (m, 2H). | |
| 98 | 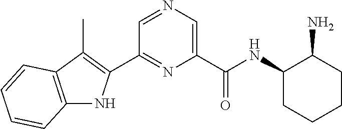 [SMILES: Cc1c(-c2cncc(C(=O)N[C@@H]3CCCC[C@@H]3N)n2)[nH]c2ccccc12] | ¹H NMR (500 MHz, DMSO-d₆) δ 12.11 (s, 1H), 9.34 (s, 1H), 9.06 (s, 1H), 8.85 (d, J = 8.3 Hz, 1H), 8.18 (s, 3H), 7.69 (d, J = 8.0 Hz, 1H), 7.53 (d, J = 8.1 Hz, 1H), 7.24 (t, J = 7.6 Hz, 1H), 7.08 (t, J = | MS m/z calcd for C₂₀H₂₃N₅O 349.2 found 350.1 [M + H]⁺ |
| | (rac)-N-((1r,2s)-2- | 7.5 Hz, 1H), 4.42-4.37 | |
| | aminocyclohexyl)-6-(3- | (m, 1H), 3.58-3.52 (m, | |
| | methyl-1H-indol-2- | 1H), 2.70 (s, 3H), 2.08- | |
| | yl)pyrazine-2-carboxamide | 2.00 (m, 1H), 1.91-1.60 | |
| | | (m, 5H), 1.56-1.43 (m, | |
| | | 2H). | |
| 99 | 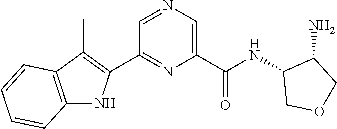 [SMILES: Cc1c(-c2cncc(C(=O)N[C@H]3COC[C@H]3N)n2)[nH]c2ccccc12] | ¹H NMR (500 MHz, DMSO-d₆) δ 12.23 (s, 1H), 9.55 (d, J = 9.0 Hz, 1H), 9.34 (s, 1H), 9.06 (s, 1H), 8.54 (s, 3H), 7.68 (d, J = 8.0 Hz, 1H), 7.57 (d, J = 8.1 Hz, 1H), 7.22 (t, J = 7.6 Hz, 1H), 7.07 (t, J = | MS m/z calcd for C₁₈H₁₉N₅O₂ 337.2 found 338.0 [M + H]⁺ |
| | (rac)-N-((3r,4s)-4- | 7.4 Hz, 1H), 5.02 (quint, | |
| | aminotetrahydrofuran-3-yl)- | J = 8.1 Hz, 1H), 4.16-4.11 | |
| | 6-(3-methyl-1H-indol-2- | (m, 3H), 4.00-3.92 (m, | |
| | yl)pyrazine-2-carboxamide | 2H), 2.69 (s, 3H). | |
| 100 | 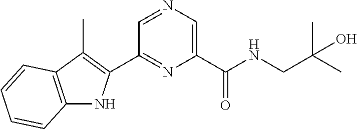 [SMILES: Cc1c(-c2cncc(C(=O)NCC(C)(C)O)n2)[nH]c2ccccc12] | ¹H NMR (500 MHz, DMSO-d₆) δ 11.78 (s, 1H), 9.31 (s, 1H), 9.04 (s, 1H), 8.93 (t, J = 6.3 Hz, 1H), 7.72-7.67 (m, 1H), 7.49 (dt, J = 8.1, 0.9 Hz, 1H), 7.25 (ddd, J = 8.1, 6.9, 1.1 Hz, 1H), 7.09 | MS m/z calcd for C₁₈H₂₀N₄O₂ 324.2 found 325.0 [M + H]⁺ |
| | N-(2-hydroxy-2- | (ddd, J = 8.0, 6.9, 1.0 Hz, | |
| | methylpropyl)-6-(3-methyl- | 1H), 3.40 (d, J = 6.4 Hz, | |
| | 1H-indol-2-yl)pyrazine-2- | 2H), 1.17 (s, 6H). | |
| | carboxamide | | |
| 101 | 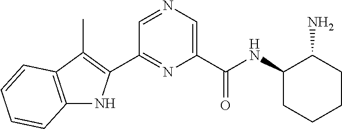 [SMILES: Cc1c(-c2cncc(C(=O)N[C@@H]3CCCC[C@H]3N)n2)[nH]c2ccccc12] | ¹H NMR (500 MHz, DMSO-d₆) δ 12.13 (s, 1H), 9.37 (d, J = 9.0 Hz, 1H), 9.31 (s, 1H), 9.04 (s, 1H), 7.98 (s, 3H), 7.69 (d, J = 8.0 Hz, 1H), 7.57 (d, J = 8.2 Hz, 1H), 7.24 (t, J = 7.6 Hz, 1H), 7.08 (t, J = | MS m/z calcd for C₂₀H₂₃N₅O 349.2 found 350.1 [M + H]⁺ |
| | (rac)-N-((1r,2r)-2- | 7.5 Hz, 1H), 4.04-3.98 | |
| | aminocyclohexyl)-6-(3- | (s, 1H), 3.52-3.44 (s, | |
| | methyl-1H-indol-2- | 1H), 2.69 (s, 3H), 2.12- | |
| | yl)pyrazine-2-carboxamide | 2.07 (m, 1H), 1.95-1.91 | |
| | | (m, 1H), 1.82-1.74 (m, | |
| | | 3H), 1.52-1.44 (m, 1H), | |
| | | 1.36-1.29 (m, 2H). | |

Example 102 & 103 were prepared in the following way:

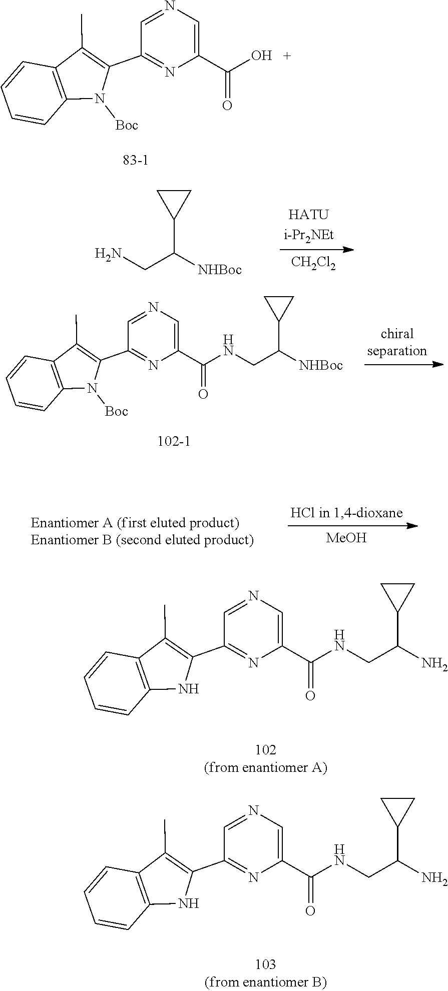

Tert-Butyl 2-(6-((2-((tert-butoxycarbonyl)amino)-2-cyclopropylethyl)carbamoyl)pyrazin-2-yl)-3-methyl-1H-indole-1-carboxylate (Compound 105-1) was prepared in a manner analogous to Example 83. LCMS (ESI) m/z calcd for C₂₉H₃₇N₅O₅ 535.3, found 536.4 (M+H)⁺. Chiral separation of racemic Compound 102-1 was performed via Preparative Chiral SFC (Stationary phase: Chiralpak AD-H, 21×250 mm, 5 μm, Mobile phase: A CO₂, B i-PrOH, Isocratic method=20% B) to yield enantiomer A as the first eluted product and enantiomer B as the second eluted product. Each enantiomer was treated with HCl in 1,4-dioxane (1 mL, 4 M) in 10 MeOH (1 mL) at rt for 3 days. After concentration, the residue was triturated with Et₂O, collected by filtration, and dried to afford Example 102 and 103 respectively.

Example 102: ¹H NMR (500 MHZ, DMSO-d₆) δ 11.99 (s, 1H), 9.69 (t, J=6.2 Hz, 1H), 9.32 (s, 1H), 9.04 (s, 1H), 8.18 (s, 3H), 7.69 (d, J=8.0 Hz, 1H), 7.53 (d, J=8.2 Hz, 1H), 7.24 (ddd, J=8.1, 6.9, 1.2 Hz, 1H), 7.08 (ddd, J=8.0, 6.9, 1.0 Hz, 1H), 3.76 (t, J=6.6 Hz, 2H), 2.73-2.70 (m, 1H), 2.69 (s, 3H), 1.05 (tt, J=8.9, 3.8 Hz, 1H), 0.67-0.54 (m, 2H), 0.51-0.46 (m, 1H), 0.43-0.38 (m, 1H). LCMS (ESI) m/z calcd for C₁₉H₂₁N₅O 335.2 found 336.2 [M+H]⁺.

Example 103: ¹H NMR (500 MHZ, DMSO-d₆) δ 11.95 (s, 1H), 9.66 (t, J=6.3 Hz, 1H), 9.33 (s, 1H), 9.04 (s, 1H), 8.16 (s, 3H), 7.69 (d, J=8.0 Hz, 1H), 7.53 (d, J=8.2 Hz, 1H), 7.24 (ddd, J=8.2, 6.9, 1.1 Hz, 1H), 7.08 (ddd, J=8.0, 6.9, 1.0 Hz, 1H), 3.76 (td, J=6.3, 5.8, 2.3 Hz, 2H), 2.73-2.70 (m, 1H), 2.69 (s, 3H), 1.11-1.00 (m, 1H), 0.66-0.54 (m, 2H), 0.51-0.46 (m, 1H), 0.43-0.37 (m, 1H). LCMS (ESI) m/z calcd for C₁₉H₂₁N₅O 335.2 found 336.2 [M+H]⁺.

Example 104 and 105 were prepared in the following way:

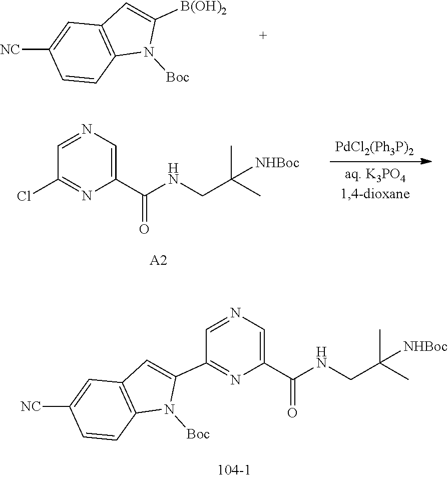

A mixture of (1-(tert-butoxycarbonyl)-5-cyano-1H-indol-2-yl) boronic acid (57 mg, 0.199 mmol, CAS 475102-15-9), Compound A2 (42 mg, 0.127 mmol), and PdCl₂(Ph₃P)₂ (4.4 mg, 0.0063 mmol) were placed in a vial. The vial was purged with N₂, and capped. To this mixture was added 1,4-dioxane (0.3 mL) and aq. K₃PO₄ (0.19 mL, 0.38 mmol, 2 M). The reaction mixture was stirred at 100° C. for 60 min. The reaction mixture was directly loaded to silica gel and purified by flash chromatography over silica gel (EtOAc/heptane gradient 0 to 50%) to afford tert-butyl 2-(6-((2-((tert-butoxycarbonyl)amino)-2-methylpropyl)carbamoyl)pyrazin-2-yl)-5-cyano-1H-indole-1-carboxylate (37 mg, 54% yield). LCMS (ESI) m/z calcd for C₂₈H₃₄N₆O₅ 534.3, found 535.2 (M+H)⁺.

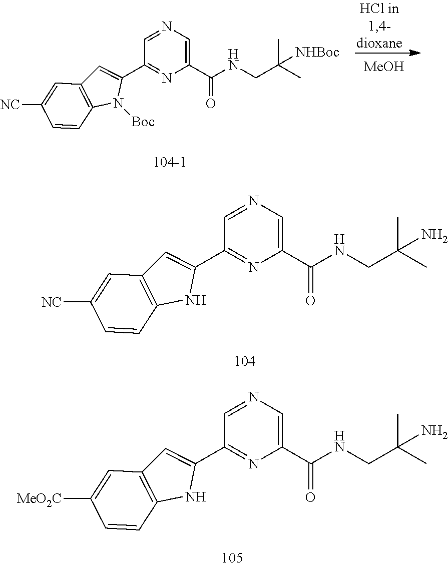 [SMILES: "MeOH" ; CC(C)(CNC(=O)c1cncc(-c2cc3cc(C#N)ccc3n2C(=O)OC(C)(C)C)n1)NC(=O)OC(C)(C)C ; CC(C)(N)CNC(=O)c1cncc(-c2cc3cc(C#N)ccc3[nH]2)n1 ; COC(=O)c1ccc2[nH]c(-c3cncc(C(=O)NCC(C)(C)N)n3)cc2c1]

To a suspension of Compound 104-1 (37 mg, 0.068 mmol) in MeOH (0.2 mL) was added HCl in 1,4-dioxane (0.7 mL, 2.8 mmol, 4 M). The reaction mixture was stirred at rt for 20 h. After the mixture was concentrated, the residue was purified by prep-HPLC to afford Example 104 (14.6 mg, 47% yield) and Example 105 (1.3 mg, 3.6% yield).

Example 104: ¹H NMR (500 MHZ, DMSO-d₆) δ 12.51 (s, 1H), 9.61 (s, 1H), 9.47 (t, J=6.7 Hz, 1H), 9.13 (s, 1H), 8.31-8.27 (m, 1H), 7.89 (s, 3H), 7.76-7.73 (m, 1H), 7.72 (dd, J=2.1, 0.9 Hz, 1H), 7.61 (dd, J=8.5, 1.6 Hz, 1H), 3.60 (d, J=6.7 Hz, 2H), 1.34 (s, 6H). LCMS (ESI) m/z calcd for C₁₈H₁₈N₆O 334.2 found 335.3 [M+H]⁺.

Example 105: ¹H NMR (500 MHZ, DMSO-d₆) δ 12.30 (s, 1H), 9.58 (s, 1H), 9.44 (t, J=7.3 Hz, 1H), 9.11 (s, 1H), 7.89 (dd, J=8.7, 1.7 Hz, 1H), 7.86 (s, 2H), 7.73 (s, 1H), 7.67 (d, J=8.7 Hz, 1H), 3.89 (s, 3H), 3.60 (d, J=6.7 Hz, 2H), 1.35 (s, 6H). LCMS (ESI) m/z calcd for C₁₉H₂₁N₅O₃ 367.2 found 368.3 [M+H]⁺.

### Example 106: N-(2-Amino-2-methylpropyl)-6-(3-chloro-5-(trifluoromethoxy)-1H-indol-2-yl)pyrazine-2-carboxamide

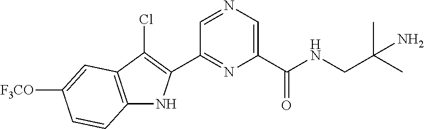 [SMILES: CC(C)(N)CNC(=O)c1cncc(-c2[nH]c3ccc(OC(F)(F)F)cc3c2Cl)n1]

The title compound was prepared in the following way:

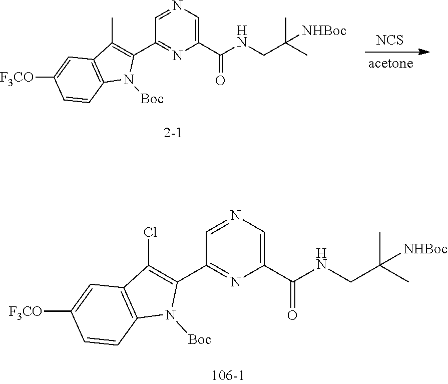 [SMILES: Cc1c(-c2cncc(C(=O)NCC(C)(C)NC(=O)OC(C)(C)C)n2)n(C(=O)OC(C)(C)C)c2ccc(OC(F)(F)F)cc12 ; "NCS" ; CC(C)(CNC(=O)c1cncc(-c2c(Cl)c3cc(OC(F)(F)F)ccc3n2C(=O)OC(C)(C)C)n1)NC(=O)OC(C)(C)C]

To a solution of Compound 2-1 (500 mg, 0.842 mmol) in acetone (5 mL) at 0° C. was added NCS (248 mg, 1.26 mmol). The reaction mixture was stirred at rt for 16 h. The reaction was quenched with aq. sat. NaHCO₃, and the mixture was extracted with EtOAc. The organic layer was concentrated in vacuo. The residue was purified by flash chromatography over silica gel (EtOAc/hexane gradient 10 to 15%) to afford tert-butyl 2-(6-((2-((tert-butoxycarbonyl)amino)-2-methylpropyl)carbamoyl)pyrazin-2-yl)-3-chloro-5-(trifluoromethoxy)-1H-indole-1-carboxylate (300 mg, 57% yield). LCMS (ESI) m/z calcd for C₂₈H₃₃ClF₃N₅O₆ 627.2, found 628.2 (M+H)⁺.

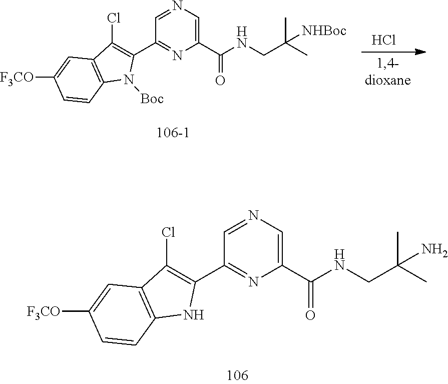 [SMILES: CC(C)(CNC(=O)c1cncc(-c2c(Cl)c3cc(OC(F)(F)F)ccc3n2C(=O)OC(C)(C)C)n1)NC(=O)OC(C)(C)C ; Cl ; CC(C)(N)CNC(=O)c1cncc(-c2[nH]c3ccc(OC(F)(F)F)cc3c2Cl)n1]

To a solution of Compound 106-1 (80 mg, 0.608 mmol) in 1,4-dioxane (1 mL) was added HCl in 1,4-dioxane (1 mL, 4 N) at 0° C. The reaction mixture was stirred at rt for 16 h. The reaction mixture was concentrated in vacuo. The residue was triturated with diethyl ether to give the crude product, which was further purified by prep-HPLC (Column: ZORBAX (150 mm×21.2 mm), 5.0), Phase A: 0.1% HCl in water, Phase B: CH₃CN (% A: 0, 2, 10, % B: 30, 35, 55) to afford the title compound (20 mg, 37% yield). ¹H NMR (500 MHZ, DMSO-d₆) δ 13.34 (s, 1H), 9.97 (t, J=6.6 Hz, 1H), 9.75 (s, 1H), 9.18 (s, 1H), 8.18 (s, 3H), 7.77 (d, J=8.9 Hz, 1H), 7.57 (d, J=2.3 Hz, 1H), 7.34 (dd, J=8.8, 2.4 Hz, 1H), 3.63 (d, J=6.6 Hz, 2H), 1.34 (s, 6H). ¹⁹F NMR (470 MHz, DMSO-d₆) δ −57.0. LCMS (ESI) m/z calcd for C₁₈H₁₇ClF₃N₅O₂ 427.1 found 428.3 [M+H]⁺.

### Example 107: N-(2-amino-2-methylpropyl)-2-(6-((2-amino-2-methylpropyl)carbamoyl)pyrazin-2-yl)-3-methyl-1H-indole-5-carboxamide

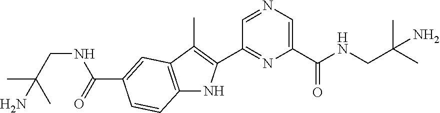 [SMILES: Cc1c(-c2cncc(C(=O)NCC(C)(C)N)n2)[nH]c2ccc(C(=O)NCC(C)(C)N)cc12]

The title compound was prepared in the following way:

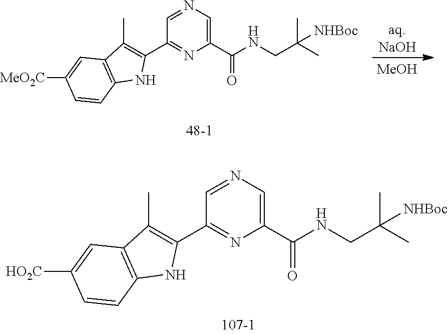 [SMILES: "MeOH" ; COC(=O)c1ccc2[nH]c(-c3cncc(C(=O)NCC(C)(C)NC(=O)OC(C)(C)C)n3)c(C)c2c1 ; Cc1c(-c2cncc(C(=O)NCC(C)(C)NC(=O)OC(C)(C)C)n2)[nH]c2ccc(C(=O)O)cc12]

To a suspension of methyl 2-(6-((2-((tert-butoxycarbonyl)amino)-2-methylpropyl)carbamoyl)pyrazin-2-yl)-3-methyl-1H-indole-5-carboxylate (Compound 48-1) (68 mg, 0.141 mmol) in MeOH (1 mL) was added aq. NaOH (0.4 mL, 0.8 mmol, 2 M). The reaction mixture was stirred at rt overnight and at 80° C. for 1.5 h. THF (1 m) was added and the reaction mixture was stirred at 80° C. for 1 h. The reaction mixture was diluted with water and Et₂O, and the resulting mixture was acidified with aq. KHSO₄. The resulting product was collected by filtration, washed with water, and dried to afford 2-(6-((2-((tert-butoxycarbonyl)amino)-2-methylpropyl)carbamoyl)pyrazin-2-yl)-3-methyl-1H-indole-5-carboxylic acid (65 mg, 99% yield). LCMS (ESI) m/z calcd for C₂₄H₂₉N₅O₅ 467.2, found 468.1 (M+H)⁺.

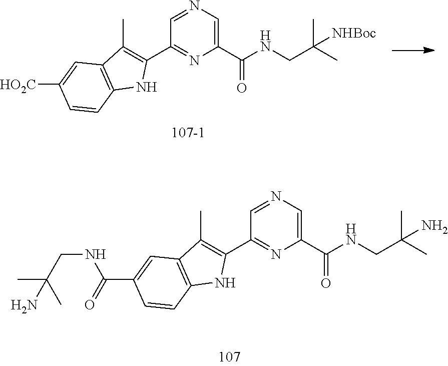 [SMILES: Cc1c(-c2cncc(C(=O)NCC(C)(C)NC(=O)OC(C)(C)C)n2)[nH]c2ccc(C(=O)O)cc12 ; Cc1c(-c2cncc(C(=O)NCC(C)(C)N)n2)[nH]c2ccc(C(=O)NCC(C)(C)N)cc12]

To a mixture of Compound 107-1 (34 mg, 0.093 mmol) and tert-butyl (1-amino-2-methylpropan-2-yl) carbamate (21 mg, 0.111 mmol) in DMF (0.4 mL) was added i-Pr₂NEt (0.032 mL, 0.185 mmol) followed by HATU (53 mg, 0.139 mmol). The reaction mixture was stirred at rt for 1.5 h. After dilution with EtOAc, the mixture was washed with aq. KHSO₄, water, sat. aq. NaHCO₃, water, and brine, dried over Na₂SO₄, filtered, and concentrated in vacuo. The residue was purified by flash chromatography over silica gel (EtOAc/heptane gradient 0 to 80%) to afford tert-butyl (1-(6-(5-((2-((tert-butoxycarbonyl)amino)-2-methylpropyl)carbamoyl)-3-methyl-1H-indol-2-yl)pyrazine-2-carboxamido)-2-methylpropan-2-yl) carbamate (LCMS (ESI) m/z calcd for C₃₃H₄₇N₇O₆ 637.4, found 638.4 (M+H)⁺), which was treated with HCl in 1,4-dioxane in MeOH at rt overnight. After concentration, the residue was triturated with MeOH—CH₃CN, collected by filtration, washed with MeOH—CH₃CN, and dried to afford the title compound (11.9 mg, 25% yield in 2 steps). ¹H NMR (500 MHZ, DMSO-d₆) δ 12.49 (s, 1H), 9.80 (t, J=6.6 Hz, 1H), 9.38 (s, 1H), 9.11 (s, 1H), 8.76 (t, J=6.3 Hz, 1H), 8.15 (d, J=1.4 Hz, 1H), 8.13 (s, 3H), 7.90 (s, 3H), 7.78 (d, J=8.5 Hz, 1H), 7.68 (dd, J=8.5, 1.5 Hz, 1H), 3.62 (d, J=6.7 Hz, 2H), 3.48 (d, J=6.2 Hz, 2H), 2.72 (s, 3H), 1.34 (s, 6H), 1.30 (s, 6H). LCMS (ESI) m/z calcd for C₂₃H₃₁N₇O₂ 437.3 found 438.2 [M+H]⁺.

### Example 108:5-(6-((2-amino-2-methylpropyl)carbamoyl)pyrazin-2-yl)-4H-thieno[3,2-b]pyrrole-2-carboxamide

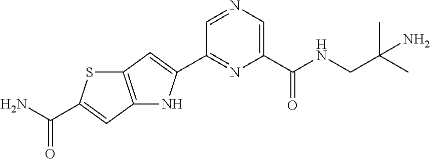 [SMILES: CC(C)(N)CNC(=O)c1cncc(-c2cc3sc(C(N)=O)cc3[nH]2)n1]

The title compound was prepared in the following way:

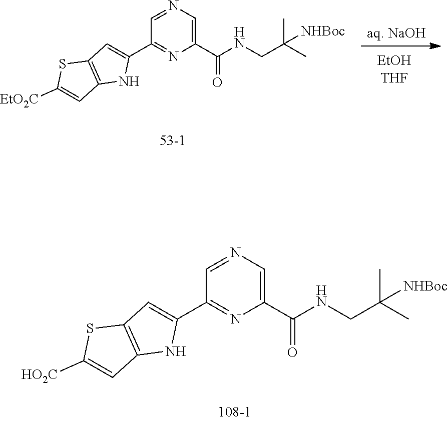 [SMILES: CCOC(=O)c1cc2[nH]c(-c3cncc(C(=O)NCC(C)(C)NC(=O)OC(C)(C)C)n3)cc2s1 ; "EtOHTHF" ; CC(C)(CNC(=O)c1cncc(-c2cc3sc(C(=O)O)cc3[nH]2)n1)NC(=O)OC(C)(C)C]

To a solution of ethyl 5-(6-((2-((tert-butoxycarbonyl)amino)-2-methylpropyl)carbamoyl)pyrazin-2-yl)-4H-thieno[3,2-b]pyrrole-2-carboxylate (Compound 53-1) (60 mg, 0.123 mmol) in EtOH (0.2 mL)-THF (0.5 mL) was added aq. NaOH (0.123 mL, 0.246 mmol, 2 M). After the reaction mixture was stirred at rt for 13 h, aq. KOH (51 mg in 0.13 mL of water) and EtOH (0.23 mL) were added. The reaction mixture was stirred at rt for 96 h. The mixture was transferred to a separate funnel using Et₂O and water. The aq. layer was separated. The Et₂O layer was extracted with water. The combined water layers were washed with Et₂O again. The aq. layer was acidified with aq. KHSO₄, and extracted with EtOAc (×2). The organic extract was dried over MgSO₄, filtered, and concentrated in vacuo to afford 5-(6-((2-((tert-butoxycarbonyl)amino)-2-methylpropyl)carbamoyl)pyrazin-2-yl)-4H-thieno[3,2-b]pyrrole-2-carboxylic acid (53 mg, 93% yield). LCMS (ESI) m/z calcd for C₂₁H₂₅N₅O₅S 459.2, found 460.2 (M+H)⁺.

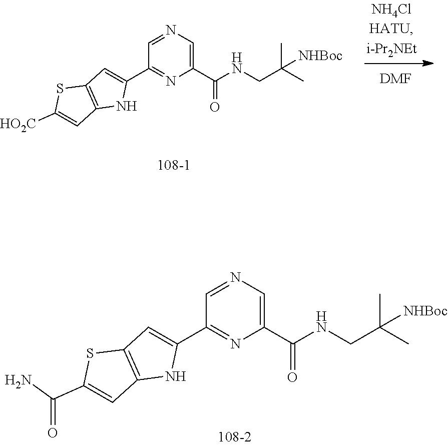

To a solution of Compound 108-1 (26 mg, 0.057 mmol) and ammonium chloride (6.1 mg, 0.113 mmol) in DMF (0.2 mL) was added i-Pr₂NEt (0.039 mL, 0.226 mmol) followed by HATU (32 mg, 0.085 mmol). The reaction mixture was stirred at rt for 30 min. After dilution with EtOAc, the mixture was washed with aq. KHSO₄, water, sat. aq. NaHCO₃, water, and brine, dried over Na₂SO₄, filtered, and concentrated in vacuo. The residue was triturated with MeOH—CH₃CN, collected by filtration, washed with CH₃CN, and dried to afford tert-butyl (1-(6-(2-carbamoyl-4H-thieno[3,2-b]pyrrol-5-yl)pyrazine-2-carboxamido)-2-methylpropan-2-yl) carbamate (23 mg, 90% yield). LCMS (ESI) m/z calcd for C₂₁H₂₆N₆O₄S 458.2, found 459.3 (M+H)⁺.

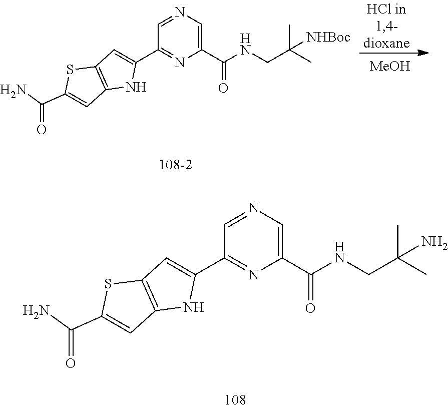 [SMILES: "MeOH" ; CC(C)(CNC(=O)c1cncc(-c2cc3sc(C(N)=O)cc3[nH]2)n1)NC(=O)OC(C)(C)C ; CC(C)(N)CNC(=O)c1cncc(-c2cc3sc(C(N)=O)cc3[nH]2)n1]

To a suspension of Compound 108-2 (23 mg, 0.050 mmol) in MeOH (0.5 mL) was added HCl in 1,4-dioxane (1 mL, 4 mmol, 4 M). The reaction mixture was stirred at rt for 30 min. The reaction mixture was concentrated in vacuo. The residue was triturated with MeOH—CH₃CN, collected by filtration, washed with MeOH—CH₃CN, and dried to afford the title compound. ¹H NMR (500 MHZ, DMSO-d₆) δ 12.54 (d, J=2.0 Hz, 1H), 9.62 (t, J=6.7 Hz, 1H), 9.38 (s, 1H), 8.97 (s, 1H), 8.09 (s, 1H), 7.97 (s, 3H), 7.85 (s, 1H), 7.51 (d, J=1.8 Hz, 1H), 7.40 (s, 1H), 3.58 (d, J=6.7 Hz, 2H), 1.33 (s, 6H). LCMS (ESI) m/z calcd for C₁₆H₁₈N₆O₂S 358.1 found 359.2 [M+H]⁺.

### Example 109: 5-(6-((2-amino-2-methylpropyl)carbamoyl)pyrazin-2-yl)-N,N-dimethyl-4H-thieno[3,2-b]pyrrole-2-carboxamide

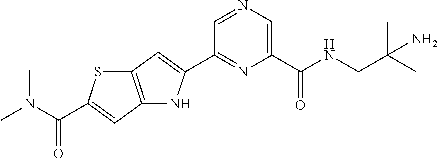 [SMILES: CN(C)C(=O)c1cc2[nH]c(-c3cncc(C(=O)NCC(C)(C)N)n3)cc2s1]

Example 109 was prepared from 5-(6-((2-((tert-butoxycarbonyl)amino)-2-methylpropyl)carbamoyl)pyrazin-2-yl)-4H-thieno[3,2-b]pyrrole-2-carboxylic acid in a manner analogous to Example 108. ¹H NMR (500 MHZ, DMSO-d₆) δ 12.51 (s, 1H), 9.57 (t, J=6.6 Hz, 1H), 9.39 (s, 1H), 8.97 (s, 1H), 7.97 (s, 3H), 7.52 (d, J=1.8 Hz, 1H), 7.49 (s, 1H), 3.58 (d, J=6.7 Hz, 3H), 3.18 (s, 6H), 1.33 (s, 6H). LCMS (ESI) m/z calcd for C₁₈H₂₂N₆O₂S 386.2 found 387.2 [M+H]⁺.

### Example 110: N-(2-amino-2-methylpropyl)-6-(5-fluoro-6-hydroxy-1H-indol-2-yl)pyrazine-2-carboxamide

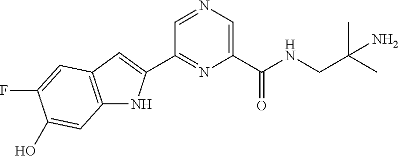 [SMILES: CC(C)(N)CNC(=O)c1cncc(-c2cc3cc(F)c(O)cc3[nH]2)n1]

The title compound was prepared in the following way:

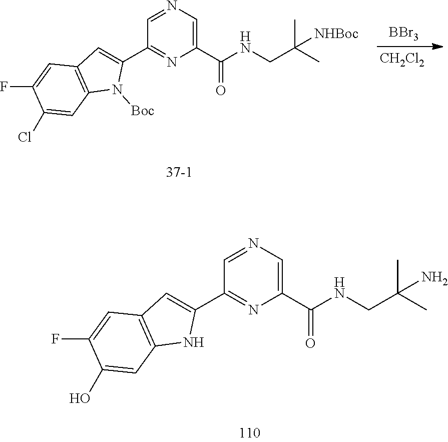

To a solution of tert-butyl 2-(6-((2-((tert-butoxycarbonyl)amino)-2-methylpropyl)carbamoyl)pyrazin-2-yl)-5-fluoro-6-methoxy-1H-indole-1-carboxylate (Compound 37-1) (60 mg, 0.131 mmol) in CH₂Cl₂ (3 mL) was added BBr₃ (0.3 mL, 0.393 mmol) at −78° C. The reaction mixture was stirred at −78° C. to rt for 16 h. The reaction was quenched with MeOH, and the mixture was concentrated in vacuo. The residue was purified by prep-HPLC (Mobile Phase: A=0.1% HCOOH in water B=CH₃CN Column: LUNA C18 (250 mm×19 mm), 4.0 μ Flow: 15 mL/min) afford the title compound. ¹⁹F NMR (376 MHZ, DMSO-d₆) δ −142.8. LCMS (ESI) m/z calcd for C₁₇H₁₈FN₅O₂ 343.1 found 344.1 [M+H]⁺.

### BIOLOGICAL ASSAYS AND DATA

The activity of a compound according to the present invention can be assessed by the following in vitro methods. A compound of formula (I), or a pharmaceutically acceptable salt thereof, exhibits valuable pharmacological properties, e.g. as indicated in tests as provided in the next sections, and are therefore indicated for therapy, e.g. in the treatment of plasmodium related diseases, e.g. malaria.

The following assay illustrates the invention without in any way limiting the scope of the invention. This parasite proliferation assay measures the increase in parasite DNA content using a DNA intercalating dye, SYBR Green®.

3D7 P. falciparum strain is grown in complete culturing media until parasitemia reaches 3% to 8% with O+ human erythrocytes. 20 μl of screening media is dispensed into 384 well assay plates. 50 nl of compounds of the invention (in DMSO), including antimalarial controls (mefloquine, pyrimethamine and artemisinin), are then transferred into the assay plates, as well as DMSO alone to serve as a negative control for inhibition. Then 30 μl of a suspension of a 3D7 P. falciparum infected erythrocytes in screening media is dispensed into the assay plates such that the final hematocrit is 2.5% with a final parasitemia of 0.3%. The plates are placed in a 37° C. incubator for 72 hours in a low oxygen environment containing 93% N₂, 4% CO₂, and 3% O₂ gas mixture. 10 μl of lysis buffer (saponin, triton-X, EDTA) containing a 10× solution of SYBR Green IR in RPMI media is dispensed into the plates. The plates are lidded and kept at room temperature overnight for the lysis of the infected red blood cells. The fluorescence intensity is measured (excitation 425 nm, emission 530 nm) using the Envision™ system (Perkin Elmer). The percentage inhibition of 50%, EC₅₀, is calculated for each compound.

Biological activity in for certain examples is represented in the table below wherein: +>EC50 0.1 μM; EC50 0.1 μM>++>EC50 0.01 μM; +++<EC50 0.01 μM.

| Compound/Example | |
|---|---|
| Number | Pf3D7 Activity |
| 1 | +++ |
| 2 | ++ |
| 3 | +++ |
| 4 | +++ |
| 5 | +++ |
| 6 | +++ |
| 7 | +++ |
| 8 | +++ |
| 9 | +++ |
| 10 | +++ |
| 11 | ++ |
| 12 | ++ |
| 13 | ++ |
| 14 | ++ |
| 15 | ++ |
| 16 | ++ |
| 17 | ++ |
| 18 | ++ |
| 19 | ++ |
| 20 | ++ |
| 21 | ++ |
| 22 | ++ |
| 23 | ++ |
| 24 | ++ |
| 25 | ++ |
| 26 | ++ |
| 27 | ++ |
| 28 | ++ |
| 29 | ++ |
| 30 | + |
| 31 | + |
| 32 | + |
| 33 | + |
| 34 | + |
| 35 | + |
| 36 | + |
| 37 | + |
| 38 | + |
| 39 | + |
| 40 | + |
| 41 | + |
| 42 | + |
| 43 | + |
| 44 | + |
| 45 | +++ |
| 46 | ++ |
| 47 | ++ |
| 48 | ++ |
| 49 | ++ |
| 50 | ++ |
| 51 | ++ |
| 52 | ++ |
| 53 | ++ |
| 54 | + |
| 54 | + |
| 56 | + |
| 57 | + |
| 58 | +++ |
| 59 | +++ |
| 60 | ++ |
| 61 | ++ |
| 62 | ++ |
| 63 | ++ |
| 64 | ++ |
| 65 | ++ |
| 66 | + |
| 67 | + |
| 68 | + |
| 69 | ++ |
| 70 | ++ |
| 71 | + |
| 72 | +++ |
| 73 | ++ |
| 74 | +++ |
| 75 | + |
| 76 | ++ |
| 77 | +++ |
| 78 | ++ |
| 79 | ++ |
| 80 | ++ |
| 81 | ++ |
| 82 | ++ |
| 83 | ++ |
| 84 | ++ |
| 85 | ++ |
| 86 | ++ |
| 87 | ++ |
| 88 | ++ |
| 89 | ++ |
| 90 | + |
| 91 | + |
| 92 | + |
| 93 | + |
| 94 | + |
| 95 | + |
| 96 | + |
| 97 | + |
| 98 | + |
| 99 | + |
| 100 | + |
| 101 | + |
| 102 | ++ |
| 103 | ++ |
| 104 | + |
| 105 | + |
| 106 | +++ |
| 107 | + |
| 108 | + |
| 109 | + |
| 110 | + |

As shown in the Tables above, compounds of the invention have on target activity. Compounds of the invention can significantly delay the increase in parasitemia.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound of formula (I), or a pharmaceutically acceptable salt thereof: 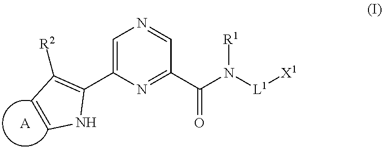
wherein:
R¹ is i) H or ii) C₁-C₃alkyl;
the moiety: 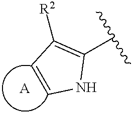
is selected from the group consisting of: 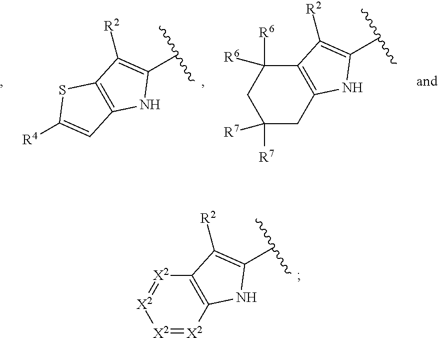
R² is i) C₁-C₃alkyl, ii) halo, iii) hydrogen, iv) C₁-C₃haloalkyl or v) cyano;
each X² is independently selected from the group consisting of N and CR³, with the proviso that at least one X² is CR³;
each R³ is independently selected from the group consisting of hydrogen, halo, SF₅, C₁-C₃alkyl, hydroxyl, cyano, O—C₁-C₃alkyl, SO₂—C₁-C₃alkyl, C(O)O—C₁-C₃alkyl, O—C₁-C₃haloalkyl, C₁-C₃haloalkyl, CH₂NH₂, OCH₂C₆H₅, C(O)—N(H)—C₁-C₄alkylene-NH₂ and 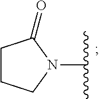 [SMILES: *N1CCCC1=O]
R⁴ is C₁-C₃alkyl, C(O)N(R⁵)₂ or CO₂C₁-C₃alkyl;
each R⁵ is independently H or C₁-C₃alkyl;
each R⁶ is H or together the two R⁶ groups form oxo;
each R⁷ is independently selected from the group consisting of H and C₁-C₃alkyl;
L¹ is i) absent or ii) C₁-C₅alkylene, optionally substituted with OH or C₃-C₆cycloalkyl;
X¹ is i) H,
ii) OH,
iii) NH₂,
iv) a) C₃-C₆cycloalkyl substituted with a NH₂ substituent, b) C₃-C₆cyclohaloalkyl substituted with a NH₂ substituent or c) 4-6 membered heterocyclyl comprising one heteroatom selected from O and N, said 4-6 membered heterocyclyl substituted with NH₂, or 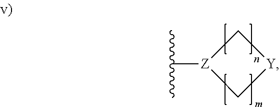
wherein Z is N or CH, Y is O or NH, n is 1 or 2 and m is 1 or 2;
provided that when L¹ is absent, X¹ is not H, OH or NH₂.

2. The compound or pharmaceutically acceptable salt thereof according to claim 1 wherein R¹ is H.

3. (canceled)

4. The compound or pharmaceutically acceptable salt thereof according to claim 1, wherein L¹ is unsubstituted C₁-C₅alkylene.

5. (canceled)

6. The compound or pharmaceutically acceptable salt thereof according to claim 1, wherein X¹ is:
iv) NH₂,
v) a) C₃-C₆cycloalkyl substituted with NH₂, b) C₃cyclohaloalkyl substituted with NH₂ or c) 4-6 membered heterocyclyl comprising one heteroatom selected from O and N, said 4-6 membered heterocyclyl being substituted with NH₂, or 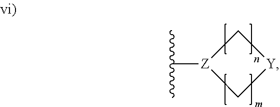
wherein Z is CH, Y is NH, n is 1 or 2 and m is 1 or 2.

7. The compound or pharmaceutically acceptable salt thereof according to claim 6 wherein X¹ is NH₂.

8. (canceled)

9. The compound or pharmaceutically acceptable salt thereof according to claim 1, wherein the moiety: 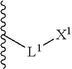
is

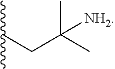 [SMILES: *CC(C)(C)N]

10. The compound or pharmaceutically acceptable salt thereof according to claim 1, wherein R² is CH₃.

11. (canceled)

12. (canceled)

13. (canceled)

14. The compound or pharmaceutically acceptable salt thereof according to claim 1, wherein the moiety: 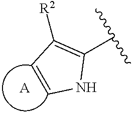
is selected from the group consisting of:

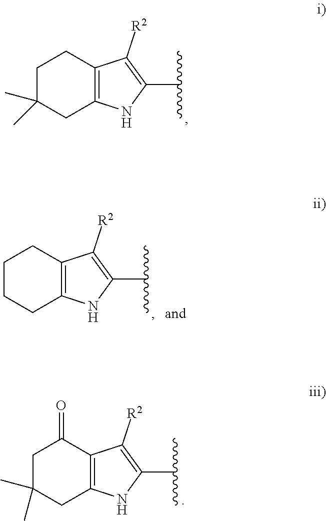

15. The compound or pharmaceutically acceptable salt thereof according to claim 1, wherein the moiety: 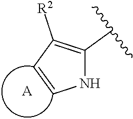
is

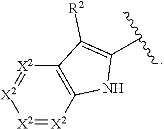

16. (canceled)

17. The compound or pharmaceutically acceptable salt thereof according to claim 1, wherein the moiety: 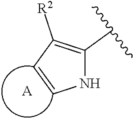
is 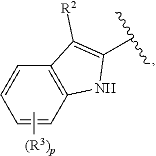
p is 1 or 2, and wherein each R³ is independently selected from the group consisting of halo, SF₅, methyl, hydroxyl, cyano, OMe, SO₂Me, C(O)OMe, O—C₁haloalkyl, C₁haloalkyl, CH₂NH₂, OCH₂C₆H₅, C(O)—N(H)—C₁-C₄alkylene-NH₂ and

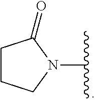 [SMILES: *N1CCCC1=O]

18. (canceled)

19. (canceled)

20. The compound or pharmaceutically acceptable salt thereof according to claim 17, wherein each R³ is independently selected from the group consisting of halo, OC₁haloalkyl, SF₅, methyl and C(O)OMe.

21. The compound or pharmaceutically acceptable salt thereof according to claim 17 wherein the moiety: 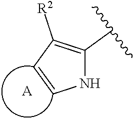
is

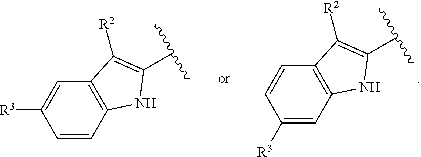

22. The compound or pharmaceutically acceptable salt thereof according to claim 21 wherein the moiety: 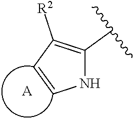
is

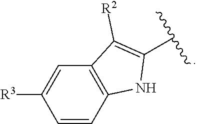

23. The compound or pharmaceutically acceptable salt thereof according to claim 21, wherein R³ is halo, OCF₃, SF₅, or OCHF₂.

24. The compound or pharmaceutically acceptable salt thereof according to claim 23 wherein R³ is SF₅.

25. The compound or pharmaceutically acceptable salt thereof according to claim 1, wherein the moiety: 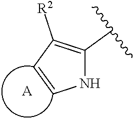
is selected from the group consisting of:

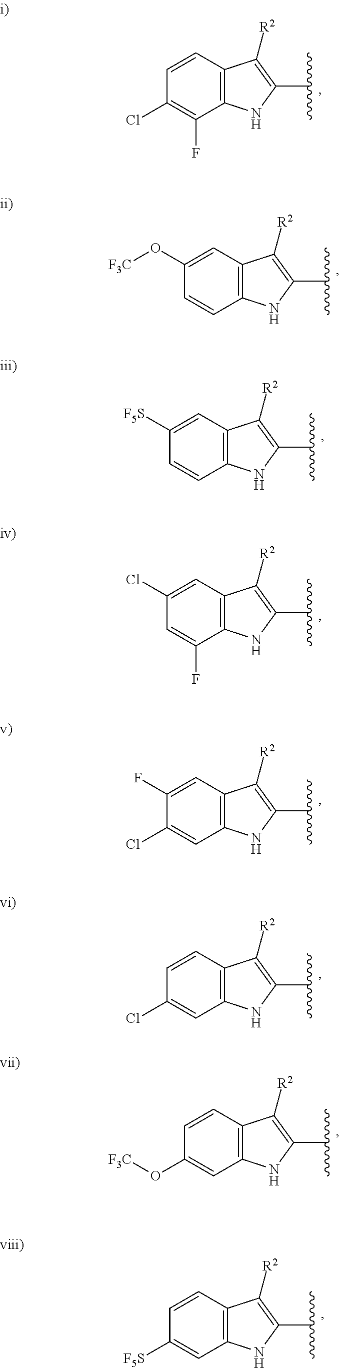

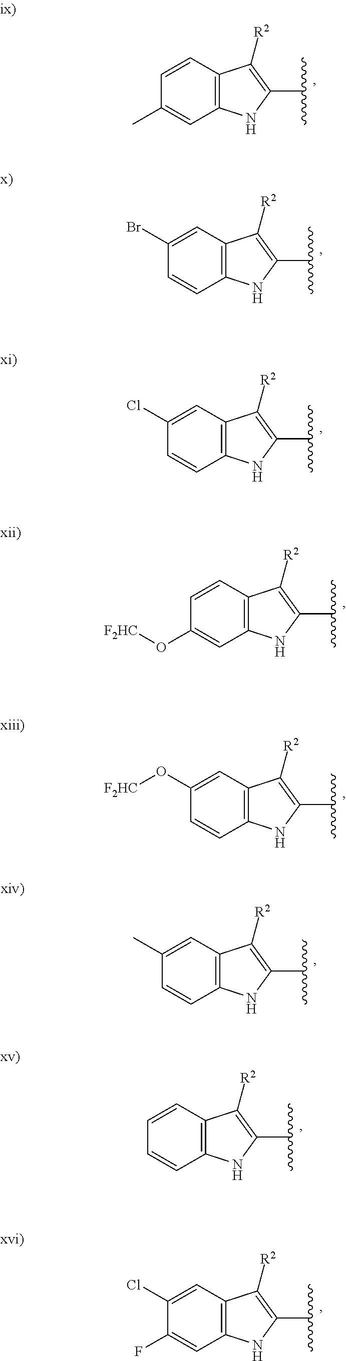

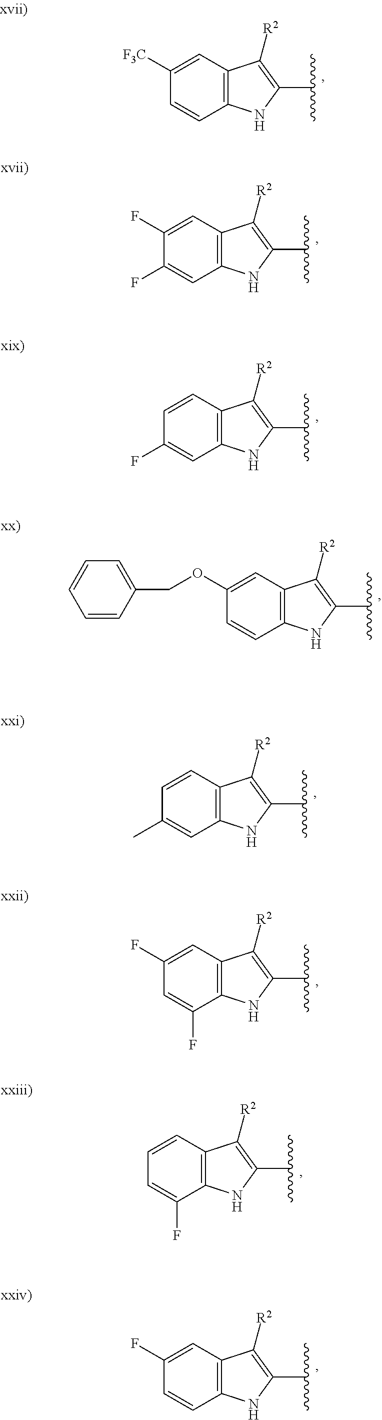

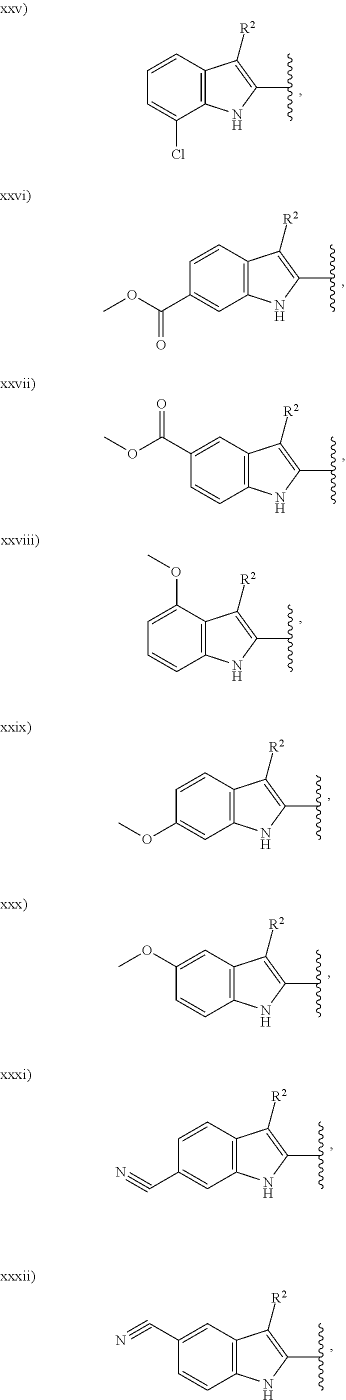

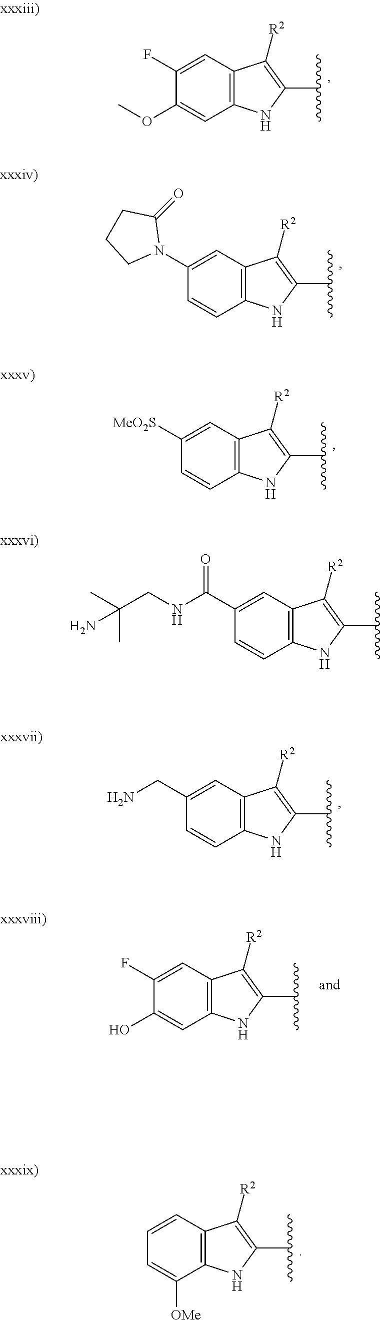

26. The compound of claim 1 selected from the group consisting of:
N-(2-amino-2-methylpropyl)-6-(6-chloro-7-fluoro-3-methyl-1H-indol-2-yl)pyrazine-2-carboxamide;
N-(2-amino-2-methylpropyl)-6-(3-methyl-5-(trifluoromethoxy)-1H-indol-2-yl)pyrazine-2-carboxamide;
N-(2-amino-2-methylpropyl)-6-(3-methyl-5-(pentafluoro-⁶-sulfaneyl)-1H-indol-2-yl)pyrazine-2-carboxamide;
N-(2-amino-2-methylpropyl)-6-(5-chloro-7-fluoro-3-methyl-1H-indol-2-yl)pyrazine-2-carboxamide;
N-(2-amino-2-methylpropyl)-6-(6-chloro-5-fluoro-3-methyl-1H-indol-2-yl)pyrazine-2-carboxamide;
N-(2-amino-2-methylpropyl)-6-(6-chloro-3-methyl-1H-indol-2-yl)pyrazine-2-carboxamide;
N-((1-aminocyclobutyl)methyl)-6-(3-methyl-5-(trifluoromethoxy)-1H-indol-2-yl)pyrazine-2-carboxamide;
N-(2-amino-2-methylpropyl)-6-(3-methyl-6-(trifluoromethoxy)-1H-indol-2-yl)pyrazine-2-carboxamide;
N-((1S,2S)-2-aminocyclopentyl)-6-(6-chloro-5-fluoro-3-methyl-1H-indol-2-yl)pyrazine-2-carboxamide;
N-(3-amino-3-methylbutyl)-6-(3-methyl-5-(trifluoromethoxy)-1H-indol-2-yl)pyrazine-2-carboxamide;
N-(2-amino-2-methylpropyl)-6-(3-methyl-6-(pentafluoro-6-sulfaneyl)-1H-indol-2-yl)pyrazine-2-carboxamide;
N-(2-amino-2-methylpropyl)-6-(5-chloro-6-fluoro-3-methyl-1H-indol-2-yl)pyrazine-2-carboxamide;
N-(2-amino-2-methylpropyl)-6-(3-chloro-5-(trifluoromethoxy)-1H-indol-2-yl)pyrazine-2-carboxamide;
N-(2-amino-2-methylpropyl)-6-(3,6-dimethyl-1H-indol-2-yl)pyrazine-2-carboxamide;
Ethyl 5-(6-((2-amino-2-methylpropyl)carbamoyl)pyrazin-2-yl)-6-methyl-4H-thieno[3,2-b]pyrrole-2-carboxylate;
N-(2-amino-2-methylpropyl)-6-(5-bromo-3-methyl-1H-indol-2-yl)pyrazine-2-carboxamide;
N-(2-(4-aminopiperidin-1-yl)ethyl)-6-(6-chloro-5-fluoro-3-methyl-1H-indol-2-yl)pyrazine-2-carboxamide;
N-(2-amino-2-methylpropyl)-6-(6-chloro-7-fluoro-1H-indol-2-yl)pyrazine-2-carboxamide;
N-(2-amino-2-methylpropyl)-6-(5-chloro-3-methyl-1H-indol-2-yl)pyrazine-2-carboxamide;
N-(2-amino-2-methylpropyl)-6-(5-chloro-7-fluoro-1H-indol-2-yl)pyrazine-2-carboxamide;
N-(2-amino-2-methylpropyl)-6-(6-(difluoromethoxy)-3-methyl-1H-indol-2-yl)pyrazine-2-carboxamide;
N-(2-amino-2-cyclopropylethyl)-6-(6-chloro-5-fluoro-3-methyl-1H-indol-2-yl)pyrazine-2-carboxamide;
N-(2-amino-2-methylpropyl)-6-(3,6,6-trimethyl-4,5,6,7-tetrahydro-1H-indol-2-yl)pyrazine-2-carboxamide;
N-(2-amino-2-methylpropyl)-6-(5-(difluoromethoxy)-3-methyl-1H-indol-2-yl)pyrazine-2-carboxamide;
N-(2-amino-2-methylpropyl)-6-(5-(pentafluoro-6-sulfaneyl)-1H-indol-2-yl)pyrazine-2-carboxamide;
N-(2-aminoethyl)-6-(3-methyl-5-(trifluoromethoxy)-1H-indol-2-yl)pyrazine-2-carboxamide;
N-(2-amino-2-methylpropyl)-6-(6-chloro-5-fluoro-1H-indol-2-yl)pyrazine-2-carboxamide;
N-(2-amino-2-methylpropyl)-6-(3,5-dimethyl-1H-indol-2-yl)pyrazine-2-carboxamide;
N-((4-aminotetrahydro-2H-pyran-4-yl)methyl)-6-(3-methyl-5-(trifluoromethoxy)-1H-indol-2-yl)pyrazine-2-carboxamide;
N-((4-aminotetrahydro-2H-pyran-4-yl)methyl)-6-(6-chloro-5-fluoro-3-methyl-1H-indol-2-yl)pyrazine-2-carboxamide;
N-(2-amino-2-methylpropyl)-6-(6-(trifluoromethoxy)-1H-indol-2-yl)pyrazine-2-carboxamide;
(S)—N-(2-amino-2-cyclopropylethyl)-6-(3-methyl-1H-indol-2-yl)pyrazine-2-carboxamide;
Methyl 2-(6-((2-amino-2-methylpropyl)carbamoyl)pyrazin-2-yl)-3-methyl-1H-indole-5-carboxylate;
(R)—N-(2-amino-2-cyclopropylethyl)-6-(3-methyl-1H-indol-2-yl)pyrazine-2-carboxamide;
N-(2-amino-2-methylpropyl)-6-(3-isopropyl-5-(trifluoromethoxy)-1H-indol-2-yl)pyrazine-2-carboxamide;
N-((1-aminocyclopropyl)methyl)-6-(6-chloro-5-fluoro-3-methyl-1H-indol-2-yl)pyrazine-2-carboxamide;
N-((1-aminocyclobutyl)methyl)-6-(3-methyl-1H-indol-2-yl)pyrazine-2-carboxamide;
N-((1-amino-3,3-difluorocyclobutyl)methyl)-6-(6-chloro-5-fluoro-3-methyl-1H-indol-2-yl)pyrazine-2-carboxamide;
N-(2-amino-2-methylpropyl)-6-(2,6-dimethyl-4H-thieno[3,2-b]pyrrol-5-yl)pyrazine-2-carboxamide;
N-(2-amino-2-methylpropyl)-6-(3-chloro-1H-indol-2-yl)pyrazine-2-carboxamide;
N-(2-amino-2-methylpropyl)-N-methyl-6-(3-methyl-5-(trifluoromethoxy)-1H-indol-2-yl)pyrazine-2-carboxamide;
N-(2-amino-2-methylpropyl)-6-(5-chloro-6-fluoro-1H-indol-2-yl)pyrazine-2-carboxamide;
N-(2-amino-2-methylpropyl)-6-(5-(trifluoromethoxy)-1H-indol-2-yl)pyrazine-2-carboxamide;
N-(2-amino-2-methylpropyl)-6-(5-(trifluoromethyl)-1H-indol-2-yl)pyrazine-2-carboxamide;
N-(2-amino-2-methylpropyl)-6-(3-methyl-1H-indol-2-yl)pyrazine-2-carboxamide;
N-(2-amino-2-methylpropyl)-6-(5-chloro-1H-indol-2-yl)pyrazine-2-carboxamide;
N-(3-aminopropyl)-6-(3-methyl-5-(trifluoromethoxy)-1H-indol-2-yl)pyrazine-2-carboxamide;
N-(3-amino-3-methylbutyl)-6-(3-methyl-1H-indol-2-yl)pyrazine-2-carboxamide;
N-(2-amino-2-methylpropyl)-6-(3-ethyl-1H-indol-2-yl)pyrazine-2-carboxamide;
N-(2-amino-2-methylpropyl)-6-(3-methyl-4,5,6,7-tetrahydro-1H-indol-2-yl)pyrazine-2-carboxamide;
N-(2-amino-2-methylpropyl)-6-(5,6-difluoro-1H-indol-2-yl)pyrazine-2-carboxamide;
N-(2-amino-2-methylpropyl)-6-(3-isopropyl-1H-indol-2-yl)pyrazine-2-carboxamide;
N-(2-amino-2-methylpropyl)-6-(6-fluoro-1H-indol-2-yl)pyrazine-2-carboxamide;
N-(3-aminobicyclo[1.1.1]pentan-1-yl)-6-(3-methyl-5-(trifluoromethoxy)-1H-indol-2-yl)pyrazine-2-carboxamide;
N-(2-amino-2-methylpropyl)-6-(5-(benzyloxy)-1H-indol-2-yl)pyrazine-2-carboxamide;
N-(2-amino-2-methylpropyl)-6-(6-methyl-1H-indol-2-yl)pyrazine-2-carboxamide;
N-(2-amino-2-methylpropyl)-6-(5,7-difluoro-1H-indol-2-yl)pyrazine-2-carboxamide;
N-(2-amino-2-methylpropyl)-6-(7-fluoro-1H-indol-2-yl)pyrazine-2-carboxamide;
N-(2-amino-2-methylpropyl)-6-(5-methyl-1H-indol-2-yl)pyrazine-2-carboxamide;
N-(2-amino-2-methylpropyl)-6-(5-fluoro-1H-indol-2-yl)pyrazine-2-carboxamide;
Ethyl 5-(6-((2-amino-2-methylpropyl)carbamoyl)pyrazin-2-yl)-4H-thieno[3,2-b]pyrrole-2-carboxylate;
N-(2-aminoethyl)-6-(3-methyl-1H-indol-2-yl)pyrazine-2-carboxamide;
N-(3-aminobicyclo[1.1.1]pentan-1-yl)-6-(3-methyl-1H-indol-2-yl)pyrazine-2-carboxamide;
N-(1-amino-2-methylpropan-2-yl)-6-(3-methyl-1H-indol-2-yl)pyrazine-2-carboxamide;
N-(2-amino-2-methylpropyl)-6-(7-chloro-1H-indol-2-yl)pyrazine-2-carboxamide;
N-((4-aminotetrahydro-2H-pyran-4-yl)methyl)-6-(3-methyl-1H-indol-2-yl)pyrazine-2-carboxamide;
Methyl 2-(6-((2-amino-2-methylpropyl)carbamoyl)pyrazin-2-yl)-1H-indole-6-carboxylate;
N-((1r,4r)-4-aminocyclohexyl)-6-(3-methyl-5-(trifluoromethoxy)-1H-indol-2-yl)pyrazine-2-carboxamide;
Methyl 2-(6-((2-amino-2-methylpropyl)carbamoyl)pyrazin-2-yl)-1H-indole-5-carboxylate;
N-(2-amino-2-methylpropyl)-6-(3-(trifluoromethyl)-1H-indol-2-yl)pyrazine-2-carboxamide;
N-(3-aminopropyl)-6-(3-methyl-1H-indol-2-yl)pyrazine-2-carboxamide;
N-(azetidin-3-yl)-6-(3-methyl-1H-indol-2-yl)pyrazine-2-carboxamide;
N-(2-amino-2-methylpropyl)-6-(4-methoxy-1H-indol-2-yl)pyrazine-2-carboxamide;
N-(2-amino-2-methylpropyl)-6-(6-methoxy-1H-indol-2-yl)pyrazine-2-carboxamide;
N-(3-aminocyclopentyl)-6-(3-methyl-1H-indol-2-yl)pyrazine-2-carboxamide;
N-(2-amino-2-methylpropyl)-6-(4,5,6,7-tetrahydro-1H-indol-2-yl)pyrazine-2-carboxamide;
N-(2-amino-2-methylpropyl)-6-(5-methoxy-1H-indol-2-yl)pyrazine-2-carboxamide;
N-(2-amino-2-methylpropyl)-6-(6-chloro-5-fluoro-1H-pyrrolo[2,3-b]pyridin-2-yl)pyrazine-2-carboxamide;
N-(2-amino-2-methylpropyl)-N-methyl-6-(3-methyl-1H-indol-2-yl)pyrazine-2-carboxamide;
N-(2-amino-2-methylpropyl)-6-(6-cyano-1H-indol-2-yl)pyrazine-2-carboxamide;
6-(3-methyl-1H-indol-2-yl)-N-(piperidin-4-yl)pyrazine-2-carboxamide;
N-(2-amino-3-hydroxypropyl)-6-(3-methyl-1H-indol-2-yl)pyrazine-2-carboxamide;
N-((3-aminooxetan-3-yl)methyl)-6-(3-methyl-1H-indol-2-yl)pyrazine-2-carboxamide;
N-(2-amino-2-methylpropyl)-6-(1H-indol-2-yl)pyrazine-2-carboxamide;
N-(2-amino-2-methylpropyl)-6-(5-cyano-1H-indol-2-yl)pyrazine-2-carboxamide;
6-(3-Methyl-1H-indol-2-yl)-N-(2-methyl-2-morpholinopropyl)pyrazine-2-carboxamide;
N-(2-amino-2-methylpropyl)-6-(5-fluoro-6-methoxy-1H-indol-2-yl)pyrazine-2-carboxamide;
N-((1r,4r)-4-aminocyclohexyl)-6-(3-methyl-1H-indol-2-yl)pyrazine-2-carboxamide;
N-(2-amino-2-methylpropyl)-6-(6-chloro-1H-pyrrolo[2,3-b]pyridin-2-yl)pyrazine-2-carboxamide;
N-((1r,4r)-4-aminocyclohexyl)-6-(5-(trifluoromethoxy)-1H-indol-2-yl)pyrazine-2-carboxamide;
N-(2-amino-2-methylpropyl)-6-(5,7-dichloro-1H-pyrrolo[2,3-c]pyridin-2-yl)pyrazine-2-carboxamide;
N-(2-amino-2-methylpropyl)-6-(5-chloro-1H-pyrrolo[2,3-c]pyridin-2-yl)pyrazine-2-carboxamide;
N-(2-amino-2-methylpropyl)-6-(6-chloro-1H-pyrrolo[3,2-c]pyridin-2-yl)pyrazine-2-carboxamide;
N-(2-amino-2-methylpropyl)-6-(3,6,6-trimethyl-4-oxo-4,5,6,7-tetrahydro-1H-indol-2-yl)pyrazine-2-carboxamide;
N-(2-amino-2-methylpropyl)-6-(4-chloro-1H-pyrrolo[3,2-c]pyridin-2-yl)pyrazine-2-carboxamide;
(rac)-N-((1r,2s)-2-aminocyclohexyl)-6-(3-methyl-1H-indol-2-yl)pyrazine-2-carboxamide;
(rac)-N-((3r,4s)-4-aminotetrahydrofuran-3-yl)-6-(3-methyl-1H-indol-2-yl)pyrazine-2-carboxamide;
N-(2-amino-2-methylpropyl)-6-(3-cyano-1H-indol-2-yl)pyrazine-2-carboxamide;
N-(2-amino-2-methylpropyl)-6-(3-methyl-5-(2-oxopyrrolidin-1-yl)-1H-indol-2-yl)pyrazine-2-carboxamide;
N-(2-amino-2-methylpropyl)-6-(3-methyl-5-(methylsulfonyl)-1H-indol-2-yl)pyrazine-2-carboxamide;
N-(2-hydroxy-2-methylpropyl)-6-(3-methyl-1H-indol-2-yl)pyrazine-2-carboxamide;
(rac)-N-((1r,2r)-2-aminocyclohexyl)-6-(3-methyl-1H-indol-2-yl)pyrazine-2-carboxamide;
6-(3-methyl-1H-indol-2-yl)-N-neopentylpyrazine-2-carboxamide;
N-(2-amino-2-methylpropyl)-6-(7-chloro-1H-pyrrolo[2,3-c]pyridin-2-yl)pyrazine-2-carboxamide;
N-(2-amino-2-methylpropyl)-6-(1H-pyrrolo[2,3-c]pyridin-2-yl)pyrazine-2-carboxamide;
5-(6-((2-amino-2-methylpropyl)carbamoyl)pyrazin-2-yl)-4H-thieno[3,2-b]pyrrole-2-carboxamide;
N-(2-amino-2-methylpropyl)-6-(5-(aminomethyl)-3-methyl-1H-indol-2-yl)pyrazine-2-carboxamide;
N-(2-amino-2-methylpropyl)-6-(5-phenyl-1H-pyrrol-2-yl)pyrazine-2-carboxamide;
N-(2-amino-2-methylpropyl)-6-(5-fluoro-6-hydroxy-1H-indol-2-yl)pyrazine-2-carboxamide;
N-(2-amino-2-methylpropyl)-6-(7-methoxy-1H-indol-2-yl)pyrazine-2-carboxamide;
5-(6-((2-amino-2-methylpropyl)carbamoyl)pyrazin-2-yl)-N,N-dimethyl-4H-thieno[3,2-b]pyrrole-2-carboxamide;
and pharmaceutically acceptable salts thereof.

27. A compound of claim 1 of formula (IIa), or a pharmaceutically acceptable salt thereof:

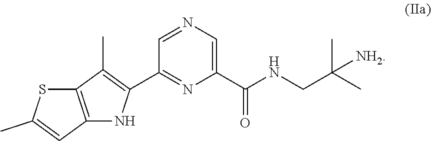 [SMILES: Cc1cc2[nH]c(-c3cncc(C(=O)NCC(C)(C)N)n3)c(C)c2s1]

28. A compound of claim 1 of formula (IIb), or a pharmaceutically acceptable salt thereof:

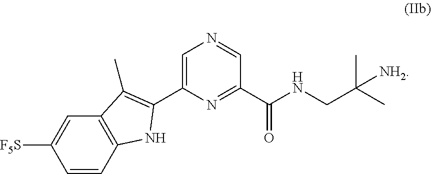

29. A compound of claim 1 of formula (IIc), or a pharmaceutically acceptable salt thereof:

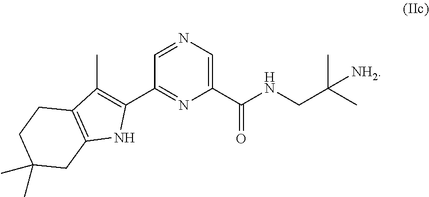 [SMILES: Cc1c(-c2cncc(C(=O)NCC(C)(C)N)n2)[nH]c2c1CCC(C)(C)C2]

30. (canceled)

31. (canceled)

32. (canceled)

33. A method of treating a Plasmodium related disease, the method comprising administering to a subject in need thereof, a therapeutically effective amount of a compound according to claim 1.

34. The method according to claim 33, wherein the Plasmodium related disease is malaria.

35. The method according to claim 33, wherein the compound is administered in combination with one or more therapeutically active agent(s).

36. The method according to claim 33, wherein the compound is administered prior to, simultaneously with, or after the therapeutically active agent.

37. The method according to claim 35, wherein the therapeutically active agent is selected from a kinase inhibitor, an anti-malarial drug and an anti-inflammatory agent.

38. The method according to claim 36, wherein the active agent is an anti-malarial drug selected from proguanil, chlorproguanil, trimethoprim, chloroquine, mefloquine, lumefantrine, atovaquone, pyrimethamine-sulfadoxine, pyrimethamine-dapsone, halofantrine, quinine, quinidine, amodiaquine, amopyroquine, sulphonamides, artemisinin, arteflene, artemether, artesunate, primaquine, pyronaridine, KAE-609, KAF-156 and INE963.

39. (canceled)

40. A pharmaceutical composition comprising the compound according to claim 1, and one or more pharmaceutically acceptable carriers.
